SEQUENCE LISTING (16 sequences)

SEQ ID NO: 1 - DNA, 20 bp, Homo sapiens
  source 1..20: mol_type = genomic DNA
atgatgcgggtctctaccag

SEQ ID NO: 2 - DNA, 20 bp, Homo sapiens
  source 1..20: mol_type = genomic DNA
ggaaccagagatctccagca

SEQ ID NO: 3 - DNA, 20 bp, Homo sapiens
  source 1..20: mol_type = genomic DNA
gcatccagctacgaatctcc

SEQ ID NO: 4 - DNA, 20 bp, Homo sapiens
  source 1..20: mol_type = genomic DNA
tcgttatcccatgtgtcgaa

SEQ ID NO: 5 - DNA, 20 bp, Homo sapiens
  source 1..20: mol_type = genomic DNA
cgcttttgggattacctgcg

SEQ ID NO: 6 - DNA, 20 bp, Homo sapiens
  source 1..20: mol_type = genomic DNA
ggggtcagttgttcctccag

SEQ ID NO: 7 - DNA, 21 bp, Homo sapiens
  source 1..21: mol_type = genomic DNA
ggatgcagaaggagatcactg

SEQ ID NO: 8 - DNA, 20 bp, Homo sapiens
  source 1..20: mol_type = genomic DNA
cgatccacacggagtacttg

SEQ ID NO: 9 - DNA, 20 bp, Homo sapiens
  source 1..20: mol_type = genomic DNA
gcgtgcaaaatggaagggtt

SEQ ID NO: 10 - DNA, 20 bp, Homo sapiens
  source 1..20: mol_type = genomic DNA
agatccgtgtcatagggcac

SEQ ID NO: 11 - DNA, 19 bp, Homo sapiens
  source 1..19: mol_type = genomic DNA
cctggccgtgtacttcctg

SEQ ID NO: 12 - DNA, 20 bp, Homo sapiens
  source 1..20: mol_type = genomic DNA
tcgctgtagacatccgacct

SEQ ID NO: 13 - DNA, 23 bp, Homo sapiens
  source 1..23: mol_type = genomic DNA
tcccaaagaagctgtgatcttca

SEQ ID NO: 14 - DNA, 20 bp, Homo sapiens
  source 1..20: mol_type = genomic DNA
tttgcttgtccaggtggtcc

SEQ ID NO: 15 - DNA, 20 bp, Homo sapiens
  source 1..20: mol_type = genomic DNA
gcatccagttggagttgaca

SEQ ID NO: 16 - DNA, 20 bp, Homo sapiens
  source 1..20: mol_type = genomic DNA
gtcctcggtcaccatagagg

(19) United States
(12) Patent Application Publication
(10) Pub. No.: US 2024/0316216 A1
(43) Pub. Date: Sep. 26, 2024

(54) LIPID NANOPARTICLES FOR DRUG DELIVERY TO MICROGLIA IN THE BRAIN

(71) Applicant: Massachusetts Institute of Technology, Cambridge, MA (US)

(72) Inventors: Robert S. Langer, Newton, MA (US); Li-Huei Tsai, Cambridge, MA (US); Owen Shea Fenton, Chaprl Hill, NC (US); Joel Blanchard, Arlington, MA (US); Jason Andresen, Andover, MA (US); Julia Bonner, Somerville, MA (US); William Ralvenius, Cambridge, MA (US)

(73) Assignee: Massachusetts Institute of Technology, Cambridge, MA (US)

(21) Appl. No.: 18/698,888

(22) PCT Filed: Oct. 6, 2022
(86) PCT No.: PCT/US2022/045909

§ 371 Date: Apr. 5, 2024

Related U.S. Application Data
(60) Provisional application No. 63/252,985, filed on Oct. 6, 2021.

(51) Int. Cl. A61K 48/00; A61K 47/14; A61K 47/28

(52) U.S. Cl. CPC A61K 48/0033; A61K 47/14; A61K 47/28; A61K 48/0058

(57) ABSTRACT

Provided herein are methods of selectively delivering an agent to a cell, comprising contacting the cell with a composition comprising an agent and lipids selected from: (a) an ionizable amino lipid, (b) a sterol, (c) a phospholipid, and (d) a PEG-lipid. The methods are selective for delivery to microglia over other neuroglia, such as astrocytes.

### CROSS-REFERENCE TO RELATED APPLICATIONS

This application claims the benefit of and priority to U.S. Provisional Application No. 63/252,985, filed Oct. 6, 2021, titled LIPID NANOPARTICLES FOR DRUG DELIVERY TO MICROGLIA IN THE BRAIN, the contents of which are incorporated herein by reference in their entirety.

### BACKGROUND OF THE INVENTION

Genetic therapies have enormous therapeutic potential. However, it is understood that the development of genetic drugs is slowed by the inability to deliver nucleic acids effectively in vivo. When unprotected, genetic materials injected into the bloodstream can be degraded by deoxyribonucleases (DNAases) and ribonucleases (RNAases), or, if not degraded, the genetic materials can stimulate an immune response. See, e.g., Whitehead et al., Nature Reviews Drug Discovery (2009) 8:129-138; Robbins et al., Oligonucleotides (2009) 19:89-102. To overcome difficulties in delivery, polynucleotides have been complexed with a wide variety of delivery systems, including polymers, lipids, inorganic nanoparticles, and viruses. See, e.g., Peer et al. Nature Nanotechnology, (2007) 2:751-760. However, despite promising data from ongoing clinical trials for the treatment of respiratory syncytial virus and liver cancers (see, e.g., Zamora et al., Am. J. Respir. Crit. Care Med. (2011) 183:531-538), the clinical use of RNA continues to require development of safer and more effective delivery systems. Toward this end, numerous lipid-like molecules have been developed including poly β-amino esters and amino alcohol lipids. See, e.g., International PCT Patent Application Publications, WO 2002/031025, WO 2004/106411, WO 2008/011561, WO 2007/143659, WO 2006/138380, and WO 2010/053572. Amino acid, peptide, polypeptide-lipids have also been studied for a variety of applications, including use as therapeutics, biosurfactants, and nucleotide delivery systems. See, e.g., Giuliani et al., Cellular and Molecular Life Sciences (2011) 68:2255-2266; Ikeda et al., Current Medicinal Chemistry (2007) 14: 111263-1275; Sen, Advances in Experimental Medicine and Biology (2010) 672:316-323; Damen et al., Journal of Controlled Release (2010) 145:33-39, WO 2013/063468; WO 2014/179562, and U.S. Publication No. 2015/0140070. Furthermore, amino acid-lipids have been found useful as delivery vehicles for messenger RNA (mRNA) therapy, which is an increasingly important option for treatment of various diseases, in particular, for those associated with deficiency of one or more proteins. See, e.g., U.S. Publication No. 2015/0140070.

Several neurodegenerative disorders are characterized by a state of neuroinflammation.¹³⁻¹⁵ Alzheimer's disease (AD) is one of the most prevalent and deadly neurological diseases requiring ongoing efforts to identify potential therapeutic remedies for the causes and symptoms of AD.^{16,17} As the major immune cells present in the brain, microglia play an outsized role in the onset or prevention of AD.¹⁸ Inflammatory microglia in the brain in AD are implicated in reduced myelination and resulting neurodegeneration. The transcription factor PU.1, controlled by the SPI1 gene in humans, is heavily involved in the regulation of functional microglia.¹⁹ Several studies have shown that overexpression of PU.1 acts as a risk factor in AD progression.^{20,21} In contrast, knockdown in PU.1. expression has been shown to be protective in AD models in vitro.^{22,23} Another major risk factor in AD involves the isoforms of the APOE gene. Specifically, ApoE4 expression is an important risk-factor leading to aberrations in lipid homeostasis and metabolism within the microglia.¹ Compared to the common ApoE3 protein, the ApoE4 variant increases risk of AD onset while the ApoE2 variant offers a neuroprotective effect.^{24,25} Further, ApoE4 lipid metabolism pathways are misregulated in microglia cells which exhibit neurodegenerative disease states such as AD.¹ These inflammatory microglia therefore serve as a potential therapeutic target to improve disease outcomes in AD or other neurodegenerative diseases.

RNA therapeutics including silencing RNA (siRNA), capable of knockdown of target genes, or messenger RNA (mRNA), allowing for inducible expression of a specific protein, could provide a therapeutic avenue to treat misregulation within the PU.1 pathway. However, due to their large size and charge, the blood-brain barrier (BBB) acts as an impediment towards delivery of RNA and other macromolecular drugs, preventing entry into the affected tissues of the brain and necessitating delivery vehicles or alternate methods of administration.^{26,27} Four-component lipid nanoparticle (LNP) systems (composed of ionizable lipid, cholesterol, phospholipid, and PEG-lipid conjugates), which receive widespread use in COVID-19 vaccines, highlight the vast potential of these delivery vehicles to deliver potential therapeutic RNA cargo.²⁸⁻³⁰ LNP transfection of hepatocytes occurs through ApoE-receptor mediated uptake.² When LNP are administered intravenously (i.v.) the corona of serum proteins which adsorb to the nanoparticle within the bloodstream directs delivery to the liver in vivo.^{31,32} Several nanoparticle systems and LNP-modifications have been designed to improve delivery across the BBB.³³⁻³⁵ Local injection of LNP has been shown to deliver reporter gene mRNA to neuronal cells and astrocytes.³⁶ Only recently have researchers identified cationic liposomes, lipid-hybrid, and polymeric nanoparticles which can be administered locally or intranasally to transfect microglia within the brain.³⁷⁻³⁹ However, clinically-relevant four-component LNP systems have not yet been established for localized RNA delivery to microglia.

However, there are currently no known methods to selectively target microglia with therapeutic drug cargo. Barriers to microglia delivery include permeating the blood-brain barrier (BBB) and poor responsiveness of microglia to traditional transfection agents. Accordingly, there is a need to investigate and develop new and improved polynucleotide delivery systems, such as ones that are more selective than existing systems.

### SUMMARY OF THE INVENTION

The present disclosure provides LNP delivery of RNA to address PU.1-mediated neuroinflammation in AD. Without wishing to be bound by any particular theory, introducing LNP formulations into the intracisternal space surrounding inflammatory microglia renders the LNP capable of transfecting microglia through ApoE-receptor pathways. Using LNP formulations with increased mRNA delivery, cells within the brain were transfected upon local injection. These LNP were then utilized to deliver anti-PU.1 siRNA to reduce neuroinflammation within mouse models of systemic or central inflammation, further validating PU.1 as a target pathway for potential therapeutic relief in AD or other neurodegenerative diseases.

In one aspect, provided herein is a method of selectively delivering an agent to a cell, comprising contacting the cell with a composition comprising the agent, and lipids selected from: (a) an ionizable amino lipid, (b) a sterol, (c) a phospholipid, and (d) a PEG-lipid. In some embodiments, the cell is a macrophage. In certain embodiments, the cell is a monocyte. In some embodiments, the cell is a microglial cell. In certain embodiments, the cell is an inflammatory microglial cell. In some embodiments, the delivery is selective for an inflammatory microglial cell over other neuroglia. In certain embodiments, the delivery is selective for an inflammatory microglial cell over an astrocyte. In certain embodiments, the agent is a polynucleotide. In some embodiments, the polynucleotide is mRNA.

In another aspect, provided herein is a method of treating or preventing a disease, disorder, or condition in a subject in need thereof, comprising administering to the subject a composition provided herein comprising an agent, for the treatment or prevention of a disease, disorder, or condition in a subject in need thereof. In some embodiments, the composition comprises an agent, and lipids selected from: (a) an ionizable amino lipid, (b) a sterol, (c) a phospholipid, and (d) a PEG-lipid. In certain embodiments, the agent is a polynucleotide. In some embodiments, the agent is mRNA. In certain embodiments, the methods of treating a disease, disorder, or condition comprise administering to the subject a therapeutically effective amount of a composition described herein. In certain embodiments, the methods of preventing a disease comprise administering to the subject a prophylactically effective amount of a composition described herein. In some embodiments, the method further comprises selectively delivering an agent to a cell. In certain embodiments, the disease, disorder, or condition that is treated or prevented by a described method is a neurological disease, inflammatory disease or condition, autoimmune disorder, cancer, or general injury. In some embodiments, the disease, disorder, or condition that is treated or prevented by a described method is a neurological disease. In some embodiments, the disease, disorder, or condition that is treated or prevented by a described method is Alzheimer's disease.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen (¹H) by deuterium (²H) or tritium (³H), replacement of ¹⁹F with ¹⁸F, or the replacement of a carbon (¹²C) by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₈ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

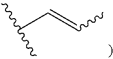

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₈ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of alkyl and heteroaryl, as defined herein, and refers to an optionally substituted alkyl group substituted by an optionally substituted heteroaryl group.

The term “unsaturated bond” refers to a double or triple bond.

The term “unsaturated” or “partially unsaturated” refers to a moiety that includes at least one double or triple bond. The term “partially unsaturated” is intended to encompass rings having multiple sites of unsaturation, but is not intended to include aromatic groups (e.g., aryl or heteroaryl moieties) as herein defined.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, —NNR^{bb}C(═O)R^{aa}, —NNR^{bb}C(═O)OR^{aa}, —NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cco}, —SO₂OR^{cco}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ee}CO₂R^{ee}, —NR^{ee}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

In certain embodiments, the one or more substituents are selected from the group consisting of halogen, —CN, —NO₂, —N₃, —SO₂H, —SO₃H, —OH, —OR^{aa}, —N(R^{bb})₂, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —S(═O)R^{aa}, —Si(R^{aa})₃, —OSi(R^{aa})₃—C(═O)SR^{a}, —OC(═O)SR^{aa}, —SC(═O)OR^{aa}, —SC(═O)R^{aa}, C₁₋₁₀ alkyl, C₂₋₁₀ alkenyl, C₂₋₁₀ alkynyl, C₃₋₆ carbocyclyl, 3-6 membered heterocyclyl, C₆ aryl, and 5-6 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R—, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphatiethioxy, heteroaliphatiethioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cco})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two RC groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R′, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{a}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR)₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such asp-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

These and other exemplary substituents are described in more detail in the Detailed Description, Examples, and Claims. The invention is not limited in any manner by the above exemplary listing of substituents.

### Other Definitions

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

As used herein, a “polymer” refers to a compound comprised of at least 3 (e.g., at least 10, 20, 30, 40, 50, 60, 70, 80, 90, 100, etc.) repeating covalently bound structural units.

“Conjugated” and “attached” refer to the covalent attachment of a group, and are used interchangeably herein.

As used herein, “lipophilic” refers to the ability of a group to dissolve in fats, oils, lipids, and lipophilic non-polar solvents such as hexane or toluene. In general, a lipophilic group refers to an unsubstituted n-alkyl or unsubstituted n-alkenyl group having 6 to 50 carbon atoms, e.g., 6 to 40, 6 to 30, 6 to 20, 8 to 20, 8 to 19, 8 to 18, 8 to 17, 8 to 16, or 8 to 15 carbon atoms.

Use of the terms “structural isomer,” “organic molecule,” and “inorganic molecule” are meant to encompass the common meaning of each term as known in the art.

As used herein, the term “small organic molecule” or “small molecule” refers to an organic molecule with a molecular weight of 1,000 g/mol or less. In certain embodiments, the molecular weight of a small molecule is at most about 1,000 g/mol, at most about 900 g/mol, at most about 800 g/mol, at most about 700 g/mol, at most about 600 g/mol, at most about 500 g/mol, at most about 400 g/mol, at most about 300 g/mol, at most about 200 g/mol, or at most about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and at most about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as an “small organometallic molecule.”

As used herein, a “large organic molecule” or “large molecule” refers to an organic compound with a molecular weight of greater than about 1,000 g/mol. In certain embodiments, the molecular weight of a large molecule is greater than about 2,000 g/mol, greater than about 3,000 g/mol, greater than about 4,000 g/mol, or greater than about 5,000 g/mol. In certain embodiments, the molecular weight of a large molecule is at most about 100,000 g/mol, at most about 30,000 g/mol, at most about 10,000 g/mol, at most about 5,000 g/mol, or at most about 2,000 g/mol. Combinations of the above ranges (e.g., greater than about 2,000 g/mol and at most about 10,000 g/mol) are also possible. In certain embodiments, the large molecule is a therapeutically active agent such as a drug (e.g., a molecule approved by the U.S. Food and Drug Administration as provided in the Code of Federal Regulations (C.F.R.)). The large molecule may also be complexed with one or more metal atoms and/or metal ions. In this instance, the large molecule is also referred to as an “large organometallic compound.”

As used herein, a “small inorganic molecule” refers to an inorganic compound with a molecular weight of 800 g/mol or less (e.g., less than 700 g/mol, less than 600 g/mol, less than 500 g/mol, less than 400 g/mol, less than 300 g/mol, less than 200 g/mol, less than 100 g/mol, between 50 to 800 g/mol, inclusive, between 100 to 800 g/mol, inclusive, or between 100 to 500 g/mol, inclusive). In certain embodiments, the small inorganic molecule is a therapeutically active agent such as a drug (e.g., a small inorganic molecule approved by the U.S. Food and Drug Administration as provided in the Code of Federal Regulations (CFR)).

As used herein, a “large inorganic molecule” refers to an inorganic compound with a molecular weight of greater than 800 g/mol (e.g., greater than 800 g/mol, greater than 900 g/mol, greater than 1000 g/mol, greater than 2000 g/mol, between 801 to 2000 g/mol, inclusive, between 900 to 2000 g/mol, inclusive, between 1000 to 2000 g/mol, inclusive, or between 801 to 1000 g/mol, inclusive). In certain embodiments, the large inorganic molecule is a therapeutically active agent such as a drug (e.g., a large inorganic molecule approved by the U.S. Food and Drug Administration as provided in the Code of Federal Regulations (CFR)).

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The terms “phosphorylethanolamine” and “phosphoethanolamine” are used interchangeably.

The term “sterol” refers to a subgroup of steroids also known as steroid alcohols, i.e., a steroid containing at least one hydroxyl group. Sterols are usually divided into two classes: (1) plant sterols also known as “phytosterols,” and (2) animal sterols also known as “zoosterols.” The term “sterol” includes, but is not limited to, cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers.

As used here, the term “PEG-lipid” refers to a PEGylated lipid.

An “amino acid” refers to natural and unnatural D/L alpha-amino acids, as well as natural and unnatural beta- and gamma-amino acids. A “peptide” refers to two amino acids joined by a peptide bond. A “polypeptide” refers to three or more amino acids joined by peptide bonds. An “amino acid side chain” refers to the group(s) pended to the alpha carbon (if an alpha amino acid), alpha and beta carbon (if a beta amino acid), or the alpha, beta, and gamma carbon (if a gamma amino acid). Exemplary amino acid side chains are depicted herein.

A “protein,” “peptide,” or “polypeptide” comprises a polymer of amino acid residues linked together by peptide bonds. The term, as used herein, refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “apolipoprotein” refers to a protein that binds a lipid (e.g., triacylglycerol or cholesterol) to form a lipoprotein. Apolipoproteins also serve as enzyme cofactors, receptor ligands, and lipid transfer carriers that regulate the metabolism of lipoproteins and their uptake in tissues. Major types of apolipoproteins include integral and non-integral apolipoproteins. Exemplary apolipoproteins include apoA (e.g., apoA-I, apoA-II, apoA-IV, and apoA-V); apoB (e.g., apoB48 and apoB 100); apoC (e.g., apoC-I, apoC-II, apoC-III, and apoC-IV); apoD; apoE; apoH; and apoJ.

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), a provirus, a lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), a polyinosinic acid, a ribozyme, a flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Such promoters include, but are not limited to: the SV40 early promoter region (Bernoist et al., Nature, 290, 304-310, (1981); Yamamoto et al., Cell, 22, 787-797, (1980); Wagner et al., Proc. Natl. Acad. Sci. U.S.A. 78, 1441-1445, (1981); Brinster et al., Nature 296, 39-42, (1982)). Any type of plasmid, cosmid, yeast artificial chromosome or viral vector can be used to prepare the recombinant DNA construct that can be introduced directly into the tissue site. Alternatively, viral vectors can be used which selectively infect the desired tissue, in which case administration may be accomplished by another route (e.g., systemically).

The polynucleotides may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “recombinant nucleic acid molecule” is a nucleic acid molecule that has undergone a molecular biological manipulation, i.e., non-naturally occurring nucleic acid molecule or genetically engineered nucleic acid molecule. Furthermore, the term “recombinant DNA molecule” refers to a nucleic acid sequence which is not naturally occurring, or can be made by the artificial combination of two otherwise separated segments of nucleic acid sequence, i.e., by ligating together pieces of DNA that are not normally continuous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in nature. Restriction enzyme recognition sites are often the target of such artificial manipulations, but other site specific targets, e.g., promoters, DNA replication sites, regulation sequences, control sequences, open reading frames, or other useful features may be incorporated by design. Examples of recombinant nucleic acid molecule include recombinant vectors, such as cloning or expression vectors which contain DNA sequences encoding Ror family proteins or immunoglobulin proteins which are in a 5′ to 3′ (sense) orientation or in a 3′ to 5′ (antisense) orientation.

The term “pDNA,” “plasmid DNA,” or “plasmid” refers to a small DNA molecule that is physically separate from, and can replicate independently of, chromosomal DNA within a cell. Plasmids can be found in all three major domains: Archaea, Bacteria, and Eukarya. In nature, plasmids carry genes that may benefit survival of the subject (e.g., antibiotic resistance) and can frequently be transmitted from one bacterium to another (even of another species) via horizontal gene transfer. Artificial plasmids are widely used as vectors in molecular cloning, serving to drive the replication of recombinant DNA sequences within host subjects. Plasmid sizes may vary from 1 to over 1,000 kbp. Plasmids are considered replicons, capable of replicating autonomously within a suitable host.

“RNA transcript” refers to the product resulting from RNA polymerase-catalyzed transcription of a DNA sequence. When the RNA transcript is a complementary copy of the DNA sequence, it is referred to as the primary transcript or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “gene silencing” refers to an epigenetic process of gene regulation where a gene is “switched off” by a mechanism other than genetic modification. That is, a gene which would be expressed (i.e., “turned on”) under normal circumstances is switched off by machinery in the cell. Gene silencing occurs when RNA is unable to make a protein during translation. Genes are regulated at either the transcriptional or post-transcriptional level. Transcriptional gene silencing is the result of histone modifications, creating an environment of heterochromatin around a gene that makes it inaccessible to transcriptional machinery (e.g., RNA polymerase and transcription factors). Post-transcriptional gene silencing is the result of mRNA of a particular gene being destroyed or blocked. The destruction of the mRNA prevents translation and thus the formation of a gene product (e.g., a protein). A common mechanism of post-transcriptional gene silencing is RNAi.

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles, macroscopic particles, pellets, crystals, aggregates, composites, pulverized, milled or otherwise disrupted matrices, and cross-linked protein or polysaccharide particles, each of which have an average characteristic dimension of about less than about 1 mm and at least 1 nm, where the characteristic dimension, or “critical dimension,” of the particle is the smallest cross-sectional dimension of the particle. A particle may be composed of a single substance or multiple substances. In certain embodiments, the particle is not a viral particle. In other embodiments, the particle is not a liposome. In certain embodiments, the particle is not a micelle. In certain embodiments, the particle is substantially solid throughout. In certain embodiments, the particle is a nanoparticle. In certain embodiments, the particle is a microparticle.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease, disorder, or condition.

As defined herein, the term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “condition,” “disease,” and “disorder” are used interchangeably.

The terms “treatment,” “treat,” and “treating” refer to reversing, alleviating, delaying the onset of, or inhibiting the progress of a disease, disorder, or condition described herein. In some embodiments, treatment may be administered after one or more signs or symptoms of the disease, disorder, or condition have developed or have been observed. In other embodiments, treatment may be administered in the absence of signs or symptoms of the disease, disorder, or condition. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

An “effective amount” of a compound described herein refers to an amount sufficient to elicit the desired biological response, i.e., treating the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound described herein is an amount sufficient to provide a therapeutic benefit in the treatment of a condition or to delay or minimize one or more symptoms associated with the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence, and which provides a prophylactic benefit in the prevention of the condition. The term “prophylactically effective amount” can encompass an amount that improves overall prophylaxis or enhances the prophylactic efficacy of another prophylactic agent.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system).

Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer. An “autoimmune disease” or “autoimmunde disorder” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “injury” refers to any physical damage to the body caused an external factor, which may be physical or chemical. In some embodiments, the injury refers to damage to the body caused by violence, accident, trauma, or fracture.

A “disease that is amenable to gene-delivery therapy” refers to any disease, disorder, or condition that involves genetic expression.

The term “neuroglia” refers to the non-neuronal cellular elements of the central and peripheral nervous systems that have a resting potential. Neuroglia have important metabolic functions, since they are invariably interposed between neurons and the blood vessels supplying the nervous system. In central nervous tissue, neuroglia include astrocytes, oligodendroglia cells, ependymal cells, and microglia cells.

The term “microglial cell” refers to the macrophage like glial cells found in the central nervous system which release pro-inflammatory substances when activated and includes mononuclear phagocytes and macrophages.

The term “astrocyte” refers to a glial cell of the nervous system. Astrocytes participate in the functioning of the blood-brain barrier, which separates the flow systemic blood of the fluids that circulate inside the brain.

The term “monocyte” refers to a mononuclear phagocyte circulating in blood that is capable of migrating into tissue and differentiating into a macrophage.

The term “macrophage” refers to refers to a phagocytic cell of mammalian tissues, derived from monocyte lineage.

### BRIEF DESCRIPTION OF THE DRAWINGS

FIGS. 1A-1C show formulation and characterization of LNP. FIG. 1A) Overall process for encapsulation and formulation of LNP. FIG. 1B) Microfluidic mixing setup utilizing PDMS chip with preformed channels. FIG. 1C) Representative DLS of cKK-E12 LNP encapsulating luciferase mRNA (n=3).

FIGS. 2A-2G show lipid nanoparticles for the introduction of mRNA into microglia. Two lines of human stem cell derived microglia (iPS and ES-microglia) cultures were assessed for their purity using flow cytometry for the microglial marker CD11b (FIG. 2A). Immunocytochemistry for the microglial marker IBA1 was used to confirm the virtually complete purity of the cultures (FIG. 2B). Comparison of LNP to lipofectamine in same cell types: transfection efficiency of mRNA packaged in LNPs was compared to lipofectamine by co-application with 50 ng mRNA coding for firefly luciferase per 10 000 cells. Student's unpaired, two-sided t-test, ****P≤0.0001. n≥3 for all conditions (FIG. 2C). Concentration-response curves for mRNA amounts and loss of viability and toxicity using the MultiTox assay: dose-response titration of luc mRNA LNP from 1 to 200 ng/mL evaluating luminescence, viability loss and toxicity in iPS (left) and ES (right) derived microglia, curves were fitted Hill's equation (FIG. 2D). LNP uptake in iPS-microglia and iPS-astrocytes under inflammatory conditions (FIG. 2E). Purity of astrocytic cultures derived from human stem cells using antibodies for astrocyte-specfic GFAP and S100B in immunocytochemistry (FIG. 2F), as well as an antibody for astrocyte-specific GLAST protein in flow cytometry (FIG. 2G).

FIG. 3 shows luciferase mRNA delivery in vitro. iPS microglia were incubated with LNP encapsulating luciferase mRNA, for 24 hours (left) and the effect of 25 ng/mL LPS treatment on LNP-mediated mRNA delivery, luminescence was normalized to delivery in the untreated cells (right).

FIG. 4 shows luciferase mRNA delivery in vivo. FIG. 4 shows a schematic demonstrating route of administration, observed luminescence in homogenized tissue from resected brain or liver samples after IP injection, and observed luminescence of resected and digested organs upon localized IC injection of luc mRNA LNP.

FIGS. 5A-5B show ranking based on luciferase delivery (luminescence) in vitro and in vivo. FIG. 5A shows the composite ranking of the various LNP formulations across in vitro and in vivo delivery performance. FIG. 5B shows the lead LNP formulation.

FIGS. 6A-6B show virtually no toxicity was detected, regardless of LNP formulation or inflammatory molecule co-application. FIG. 6A shows cell viability and FIG. 6B shows detailed toxicity experiments in iPS microglia as measured by Promega Multitox assay. Cells were treated with immunostimulatory signals prior to LNP treatment. ANOVA, Dunnett's post hoc against no LNP treatment. ***P≤0.001. n≤4 for all conditions.

FIGS. 7A-7B show no luciferase expression was detected in blood plasma. Collection of blood from mice for observed luminescence after IP (FIG. 7A) or IC (FIG. 7B) administration of LNP. ANOVA, Dunnett's post hoc against No LNP treatment. n≤3 for all conditions.

FIG. 8 shows the effect of pro-inflammatory stimulation. Observed raw luminescence units for initial LNP formulation screen in iPS microglia. Cells were treated with immunostimulatory signals prior to LNP treatment. ANOVA, Dunnett's post hoc against No LNP treatment. **P≤0.01, *P≤0.05. n≤4 for all conditions. n≤4 for all conditions.

FIG. 9 shows flow cytometry and immunocytochemistry confirming a microglial identity of iPS and ES derived microglia.

FIG. 10 shows flow cytometry and immunocytochemistry confirming an astrocytic identify of the iPS derived astrocytes.

FIG. 11 shows dissection diagram, performed one day after injection of lipid nanoparticles with luciferase mRNA.

FIG. 12 shows comparisons of lipid nanoparticle distribution in the mouse. Seven-month-old wildtype littermates and P301STau mice were injected with LNPs intraperitoneally or intracisternally. One day later, peripheral organs (spleen, kidney, liver, lung) and brain were resected, or peripheral body parts (eye, ear and tongue) as well as different brain regions were dissected. Luciferase expression was quantified.

FIG. 13 shows LNP formulation variations focused on lipid:mRNA ratio, ionizable lipid, and phospholipid composition, as well as the structure of ionizable lipid cKK-E12.

FIG. 14A shows the comparison of luminescence (left) and cell number (right) upon LNP delivery in iPS microglia (left grey bars) and iPS astrocytes (right dark bars) after administration of immunostimulants, LPS or PMA. Student's unpaired, two-sided t-test, ****P≤0.0001. n≥3 for all conditions. FIG. 14B shows internal organs and FIG. 14C shows regions of the brain after IP or IC delivery of luc mRNA-LNP into wildtype, C57BL/6J mice. Organs were resected and digested prior to luminescence measurements. Student's unpaired, two-sided t-test. n≥6 for all conditions.

FIGS. 15A-15H show the delivery of anti-PU.1 siRNA with LNPs in human iPS microglia. FIG. 15A shows the concentration-response curve in RTqPCR experiments—showing SPI1 levels (the gene for PU.1) normalized to ACTB—comparing delivery of siRNA with RNAiMAX lipofectamine (circles) and LNPs (triangles). Curves were fitted Hill's equation. FIG. 15B shows the results of RTqPCR experiments on various genes (IL1B—triangles, TNFA—stars, TREM2—grey circles, SPI1—black circles), normalized to ACTB, over time after delivery of PU.1 siRNA using LNPs. FIG. 15C shows a time course of cell death (grey circles) after PU.1 silencing (black circles). Cell death was measured with CellTiterGlo. FIG. 15D shows RTqPCR after delivery of anti-PU.1 siRNA, side-by-side in iPS (left) and ES (right) derived microglia, normalized to ACTB. Student's unpaired, two-sided t-test performed on cycle numbers, ****P≤0.0001, ***P≤0.001, n≥3 for all data points. Bars are mean±SEM, each data point represents one well. FIG. 15E shows immunocytochemistry of iPS microglia treated with LNPs carrying either scrambled siRNA (left) or PU.1 siRNA (right), staining for cytoplasmic IBA1 and nuclear PU.1, counterstained with Hoechst. FIG. 15F shows flow cytometry using a PU.1 antibody, comparing RNAiMAX and LNPs containing either scramble or anti-PU.1 siRNA. Student's unpaired, two-sided t-test performed on cycle numbers, ****P≤0.0001, **P≤0.01. Bars are mean±SEM, each data point represents a well. FIG. 15G shows the concentration response curve, comparing delivery with RNAiMAX Lipofectamine and LNPs. FIG. 15H shows quantification at 500 ng/mL. Student's unpaired, two-sided t-test.

FIGS. 16A-16I show the silencing of PU.1 with LNPs is anti-inflammatory during acute systemic inflammation. FIG. 16A shows an illustration of a BV2 (immortalized mouse microglia) cell line in which a PU.1 activity reporter construct was incorporated into the Rosa26 locus. FIG. 16B depicts the concentration response curve of adding LNPs with increasing amounts of either scramble non-targeting siRNA (black circles) or anti-PU.1 siRNA (grey circles). Curves were fitted Hill's equation. FIG. 16C shows a schematic of in vivo mouse experiments. LPS was injected at 10 mg/kg, followed by an injection that was either intracisternal (top) or intraperitoneal (bottom), containing LNPs with either scrambled siRNA (open bars) or anti-PU.1 siRNA (black filled bars). One day later, mice were placed in the Open Field arena and had their general locomotor activity measured over 10 minutes. Student's unpaired, two-sided t-test. FIG. 16D shows liver sections stained with hematoxylin & eosin after IC (top) or IP injection (bottom) of LNP encapsulating scramble or anti-PU.1 siRNA. LPS injected mice exhibited red-stained vessels, caused by accumulation of immune cells in vessels, and are indicated with black arrows. Vessels were considered to be intact, circular and empty structures of approximately 40 m size. Scale bar is 40 m. White arrows indicate vessels not containing accumulated immune cells. Student's unpaired, two-sided t-test. FIG. 16E shows comparison levels of Sfp1, Il1b, and Ccl2 mRNA transcripts as determined by RTqPCR in whole brain, liver, and spleen tissue after IP LPS administration. Student's unpaired, two-sided t-test. FIG. 16F shows confocal micrographs of hippocampal tissue stained for PU.1-positive nuclei and all nuclei with Hoechst in LPS injected mice, co-injected intraperitoneally (i.p.) with either scrambled siRNA (top) or anti-PU.1 siRNA (bottom) and quantification of untreated (no LPS or LNP injection) mice in light grey bars (n=3). i.p. is intraperitoneal and i.c.st. is intracisternal injection of LNPs. LPS-injected mice co-injected with LNPs containing scramble (empty bars, n=6 for i.p. and 9 for i.c.st.) or anti-PU.1 siRNA (black bars, n=6 for i.p. and 6 for i.c.st). Student's unpaired, two-sided t-test. Scale bar is 50 m. FIGS. 16G-16I show confocal micrographs of hippocampal tissue stained for IBA1 (FIG. 16G), C1Q (FIG. 16H), and GFAP (FIG. 16I) and stained for immunoreactive area.

FIGS. 17A-17D show silencing of PU.1 with LNPs during chronic neuroinflammation and neurodegeneration in CKp25 mice. FIG. 17A shows the time-course of luminescence in different organs after a single intracisternal injection of LNPs with luciferase mRNA. Each data point represents an average of 4 mice for all conditions, error bars are ±SEM. FIG. 17B shows the timeline of experiments in CKp25 mice. Mice were induced for two weeks with p25 and injected with anti-PU.1 siRNA LNP on days 0, 3, 6, 9 and 12. FIG. 17C shows confocal micrographs of hippocampal tissue of, from left to right, CK mice, CKp25 mice treated with LNPs containing vehicle, or CKp25 mice treated with LNPs containing anti-PU.1 siRNA. FIG. 17D shows quantification of, from top to bottom, number of PU.1+nuclei, surface area stained for IBA1, C1Q, or GFAP.

FIGS. 18A-18E show neuroinflammation caused by LPS in mice. FIGS. 18A-18C show a schematic of in vivo mouse experiments. LPS was injected at 10 mg/kg (FIG. 18A). One day later, mice were placed in the Open Field arena and had their general locomotor activity measured over 10 minutes (FIG. 18B). Student's unpaired, two-sided t-test. Liver sections stained with hematoxylin and eosin. LPS injected mice (FIG. 18C) exhibited red-stained vessels, caused by accumulation of immune cells in vessels, and are indicated with black arrows. Vessels are considered to be intact, circular and empty structures of approximately 40 m size. Scale bare is 40 m. White arrows indicate vessels not containing accumulated immune cells. FIG. 18D shows confocal micrographs of hippocampal tissue of saline (left) or LPS (right) injected mice. FIG. 18E shows quantification of, from top to bottom, number of PU.1+nuclei, surface area stained for IBA1, or GFAP. Student's unpaired, two-sided t-test. n=10 for saline and 13 for LPS injected mice.

FIGS. 19A-19B show the comparison of iPS microglia/astrocytes in scrambled siRNA against iPS microglia/astrocytes in PU.1 siRNA. FIG. 19A shows the time course of luciferase expression in iPS microglia. n≤8 for all conditions. FIG. 19B shows the luminescence and viability of cells that were treated with 5-methoxyuridine and mRNA that was not modified. The 5-methoxyuridine modification was necessary for efficacious expression of mRNA in iPS microglia.

### DETAILED DESCRIPTION OF CERTAIN EMBODIMENTS

Provided herein are methods of selectively delivering to a cell a composition comprising an ionizable amino lipid, a sterol, a phospholipid, and a PEG-lipid provided herein. Such methods of selective delivery are useful for the treatment and/or prevention of various diseases, such as neurological diseases.

### Methods

In one aspect, provided herein is a method of selectively delivering an agent to a cell, comprising contacting the cell with a composition comprising the agent, and lipids selected from: (a) an ionizable amino lipid, (b) a sterol, (c) a phospholipid, and (d) a PEG-lipid. In certain embodiments, the cell is a macrophage. In some embodiments, the cell is a monocyte. In certain embodiments, the cell is a microglial cell. In some embodiments, the cell is an inflammatory microglial cell. In certain embodiments, the delivery is selective for an inflammatory microglial cell over one or more other neuroglia. In some embodiments, the delivery is selective for an inflammatory microglial cell over one other neuroglia. In certain embodiments, the delivery is selective for an inflammatory microglial cell over an astrocyte.

In another aspect, provided herein is a method of treating or preventing a disease, disorder, or condition in a subject in need thereof, comprising administering to the subject a composition provided herein comprising an agent, for the treatment or prevention of a disease, disorder, or condition in a subject in need thereof. In some embodiments, the composition comprises an agent, and lipids selected from: (a) an ionizable amino lipid, (b) a sterol, (c) a phospholipid, and (d) a PEG-lipid. In certain embodiments, the agent is a polynucleotide. In some embodiments, the agent is mRNA.

In certain embodiments, the methods of treating a disease, disorder, or condition comprise administering to the subject a therapeutically effective amount of a composition described herein. In certain embodiments, the methods of preventing a disease, disorder, or condition comprise administering to the subject a prophylcatically effective amount of a composition described herein.

In some embodiments, the method further comprises selectively delivering an agent to a cell. In certain embodiments, the cell is a macrophage. In some embodiments, the cell is a monocyte. In certain embodiments, the cell is a microglial cell. In some embodiments, the cell is an inflammatory microglial cell. In certain embodiments, the delivery is selective for an inflammatory microglial cell over one or more other neuroglia. In some embodiments, the delivery is selective for an inflammatory microglial cell over one other neuroglia. In certain embodiments, the delivery is selective for an inflammatory microglial cell over an astrocyte.

In some embodiments, the subject is a mammal. In certain embodiments, the subject is a human.

In some embodiments, the disease, disorder, or condition that is treated or prevented by a described method is a disease that is amenable to gene-delivery therapy. In some embodiments, the disease, disorder, or condition that is treated or prevented by a described method is a neurological disease, an inflammatory disease or condition, an autoimmune disorder, cancer, or an injury.

In certain embodiments, the disease, disorder, or condition that is treated or prevented by a described method is a neurological disease. In some embodiments, the disease that is treated or prevented by a described method is a neurodegenerative disease. In some embodiments, the disease that is treated or prevented by a described method is Alzheimer's disease or other dementias, Parkinson's disease or Parkinson's disease-related disorders, Huntington's disease, Amyotrophic Lateral Sclerosis (ALS), prion diseases, corticobasal degeneration, frontotemporal dementia, HIV-related cognitive impairment, mild cognitive impairment, motor neuron diseases, spinocerebellar ataxia, spinal muscular atrophy, Friedreich's ataxia, Lewy body disease, Alpers' disease, Batten disease, cerebro-oculo-facio-skeletal syndrome, corticobasal degeneration, Gerstmann-Straussler-Scheinker disease, Kuru, Leigh's disease, monomelic amyotrophy, multiple system atrophy, multiple system atrophy with orthostatic hypotension (Shy-Drager syndrome), multiple sclerosis (MS), neurodegeneration with brain iron accumulation, opsoclonus myoclonus, posterior cortical atrophy, primary progressive aphasia, progressive supranuclear palsy, vascular dementia, progressive multifocal leukoencephalopathy, dementia with Lewy bodies, lacunar syndromes, hydrocephalus, Wernicke-Korsakoff's syndrome, post-encephalitic dementia, cancer and chemotherapy-associated cognitive impairment and dementia, or depression-induced dementia and pseudodementia. In certain embodiments, the disease that is treated or prevented by a described method is Alzheimer's disease.

In some embodiments, the disease, disorder, or condition that is treated or prevented by a described method is an inflammatory disease or condition. In certain embodiments, the inflammatory disease or condition that is treated or prevented by a described method is generalized inflammation. In some embodiments, the inflammatory disease or condition that is treated or prevented by a described method is pathogen-associated inflammation. In certain embodiments, the pathogen-associated inflammation is one or more of sepsis, bacterial injection, viral infection, SARS-CoV-2 infection, parasite infection, helminth infection, Epstein-Barr infection, hepatitis B, hepatitis C, or human papillomavirus infection. In some embodiments, the inflammatory condition is peripheral inflammation. In certain embodiments, the peripheral inflammation is associated with one or more of rheumatoid arthritis, colitis, prostatitis, carditis, vasculitis, lymphangitis, lymphadenitis, insulitis, hypophysitis, thryoiditis, parathyroiditis, adrenalitis, funisitis, omphalitis, chorioamnionitis, oophoritis, salpingitis, endometritis, parametritis, cervicitis, vaginitis, vulvitis, mastitis, orchitis, epididymitis, seminal vesiculitis, balanitis, posthitis, balanoposthitis, glomerulonenephritis, pyelonephritis, dermatomyositis, synovitis, bursitis, fasciitis, tendinitis, panniculitis, spondylitis, periostitis, capsulitis, enthesitis, tenosynovitis, chondritis, dermatitis, cellulitis, hidradenitis, hepatitis, cholangitis, cholecystitis, pancreatitis, peritonitis, proctitis, appendicitis, casecitis, ileitis, duodenitis, enterocolitis, colitis, enteritis, gastroenteritis, gastritis, esophagitis, stomatitis, gingivitis, gengivostomatitis, glossitis, tonsillitis, parotitis, cheilitis, sialadenitis, gnathitis, sinusitis, rhinitis, pharyngitis, laryngitis, tracheitis, bronchitis, bronchiolitis, pneumonitis, pleuritis, endocarditis, myocarditis, pericarditis, arteritis, phlebitis, capillaritis, neuritis, otitis media, otitis externa, labyrinthitis, mastoiditis, uveitis, conjunctivitis, blepharitis, chorioretinitis, retinitis, keratitis, scleritis, dacyoadenitis, arachnoiditis, meningitis, myelitis, encephalitis, skin burns, sun burn, frostbite, skin laceration, ionizing radiation, anaphylactic shock, onychocryptosis, acne vulgaris, asthma, celiac disease, diverticulitis, Familial Mediterranean Fever, Glomerulonephritis, sarcoidosis, transplant rejection, vasculitis, reperfusion injury, otitis, licen planus, Mast Cell Activation Syndrome, Hidradenitis suppurativa, Inflammatory bowel syndrome, Vitamin A deficiency, insomnia, Chediak-Higashi syndrome, fever, edema, malaise, fibrosis, abscess formation, gum disease, periodontitis, hay fever, pollen allergy, bee venom allergy, animal allergy, nut allergy, food allergy, dust allergy, toxin induced inflammation, foreign body/debris induced inflammation, herpes simplex infection, herpes simplex flare.

In some embodiments, the disease, disorder, or condition that is treated or prevented by a described method is an autoimmune disorder. In certain embodiments, the autoimmune disorder that is treated or prevented by a described method is one or more of systemic lupus erythematosus, aplastic anemia, rheumatoid arthritis, inflammatory bowel disease, coeliac disease, lactose intolerance, type I diabetes, type II diabetes, alopecia areata, angioedema, Autoimmune urticaria, Bullous pemphigoid, Dermatitis herpetiformis, Amyloidosis, Agammaglobulinemia, Eczema, Dego's disease, Contact dermatitis, Congenital heart block, Complement component 2 deficiency, Castleman's disease, Sweet's syndrome, Takayasu's arteritis, Serum sickness, Rasmussen's encephalitis, IPEX syndrome, Blau syndrome, Autoimmune polyendocrine syndrome (APS) type 1 and 2 and 3, Autoimmune pancreatitis, Autoimmune thyroiditis, Ord's thyroiditis, Grave's disease, Autoimmune oophoritis, Endometriosis, Autoimmune orchitis, Sjögren syndrome, Autoimmune enteropathy, Crohn's disease, ulcerative colitis, Cold agglutinin disease, Autoimmune neutropenia, Autoimmune lymphoproliferative syndrome, Aplastic anemia, Antiphospholipid syndrome, Autoimmune hemolytic anemia, Lyme disease, chronic Lyme disease, Mixed connective tissue disease, Schnitzler syndrome, Fibromyalgia, myositis, Myasthenia gravis, neuromyotonia, polymyositis, Balo concentric sclerosis, Guillain-Barre syndrome, Lambert-Eaton myasthenic syndrome, Idiopathic inflammatory demyelinating diseases, Hashimoto's encephalopathy, Restless legs syndrome, Stiff-person syndrome, Sydenham's chorea, or Pediatric Autoimmune Neuropsychiatric Disorder Associated with Streptococcus.

In some embodiments, the disease, disorder, or condition that is treated or prevented by a described method is cancer. In some embodiments, the cancer that is treated or prevented by a described method is one or more of glioblastoma, osteosarcoma, thyroid cancer, bone cancer, carcinoma, sarcoma, lymphoma, leukemia, germ cell tumor, blastoma, cervical cancer, ultraviolet radiation induced cancer, melanoma, radon induced cancer, smoking induced cancer, lung cancer, stomach cancer, breast cancer, or colorectal cancer.

In certain embodiments, the disease, disorder, or condition that is treated or prevented by a described method is an injury. In some embodiments, the injury that is treated or prevented by a described method is one or more of laceration, flesh wounds, trauma, bone break, skin laceration, or internal bleeding.

### (a) Ionizable Amino Lipid

### Formula (I)

In some embodiments, the ionizable amino lipid is a compound of Formula (I):

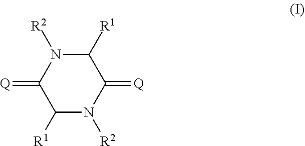

or salt thereof;
wherein:
each instance of Q is independently O, S, or NR^{Q}, wherein R^{Q} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), (iii);
each instance of R¹ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, halogen, —OR^{A1}, —N(R^{A1})₂, or —SR^{A1}; wherein each occurrence of R^{A1} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to an sulfur atom, a nitrogen protecting group when attached to a nitrogen atom, or two R^{A1} groups are joined to form an optionally substituted heterocyclic or optionally substituted heteroaryl ring; and
each instance of R² is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii); and Formulae (i), (ii), and (iii) are:

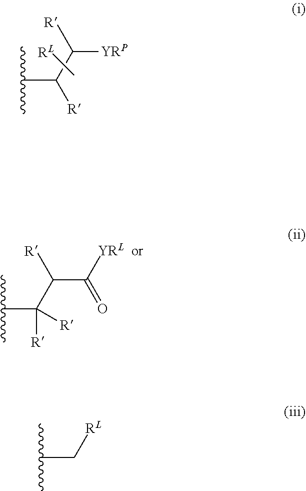

wherein:
each instance of R′ is independently hydrogen or optionally substituted alkyl;
X is O, S, NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
Y is O, S, NR, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and
R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer; provided that at least one instance of R^{Q}, R², or R¹ comprise a group of the formula (i), (ii), or (iii).

The following chemical group definitions and embodiments apply to compounds of Formula (I) and all subgenera thereof provided herein.

Compounds of Formula (I) are the cyclic condensation product of the same or different two amino acids, and further comprise one or more sites of conjugation attached thereto, e.g., to an internal amide nitrogen, to an amino substituent, and/or to an imino nitrogen, of a group of formula (i), (iii), or (iii). Such groups may be conjugated before cyclization, i.e., to the amino acid precursors of the cyclization product, or after cyclization.

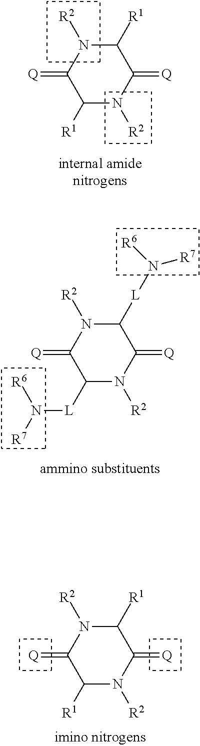

As generally defined above, each instance of R′ is independently hydrogen or optionally substituted alkyl. In certain embodiments, at least one instance of R′ is hydrogen. In certain embodiments, at least two instances of R′ is hydrogen. In certain embodiments, each instance of R′ is hydrogen. In certain embodiments, at least one instance of R′ is optionally substituted alkyl, e.g., methyl. In certain embodiments, at least two instances of R′ is optionally substituted alkyl, e.g., methyl. In certain embodiments, one instance of R′ is optionally substituted alkyl, and the rest are hydrogen.

As generally defined above, each instance of Q is independently O, S, or NR^{Q}, wherein R^{Q} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii). In certain embodiments, at least one instance of Q is O. In certain embodiments, each instance of Q is O. In certain embodiments, at least one instance of Q is S. In certain embodiments, each instance of Q is S. In certain embodiments, at least one instance of Q is NR^{Z}. In certain embodiments, each instance of Q is NR^{Z}. In certain embodiments, each instance of R^{Q} is independently hydrogen or a group of the formula (i), (ii), or (iii).

As generally defined above, each instance of R¹ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, halogen, —OR^{A1}, —N(R^{A1})₂, or —SR^{A1}.

In certain embodiments, at least one instance of R¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl.

In certain embodiments, at least one instance of R¹ is optionally substituted alkyl; e.g., optionally substituted C₁₋₆alkyl, optionally substituted C₂₋₆alkyl, optionally substituted C₃₋₆alkyl, optionally substituted C₄₋₆alkyl, optionally substituted C₄₋₅alkyl, or optionally substituted C₃₋₄alkyl.

In certain embodiments, at least one instance of R¹ is optionally substituted alkenyl, e.g., optionally substituted C₂₋₆alkenyl, optionally substituted C₃₋₆alkenyl, optionally substituted C₄₋₆alkenyl, optionally substituted C₄₋₅alkenyl, or optionally substituted C₃₋₄alkenyl.

In certain embodiments, at least one instance of R¹ is optionally substituted alkynyl, e.g., optionally substituted C₂₋₆alkynyl, optionally substituted C₃₋₆alkynyl, optionally substituted C₄₋₆alkynyl, optionally substituted C₄₋₅alkynyl, or optionally substituted C₃₋₄alkynyl.

In certain embodiments, at least one instance of R¹ is optionally substituted carbocyclyl, e.g., optionally substituted C₃₋₁₀ carbocyclyl, optionally substituted C₅₋₈ carbocyclyl, optionally substituted C₅₋₆ carbocyclyl, optionally substituted C₅ carbocyclyl, or optionally substituted C₆ carbocyclyl.

In certain embodiments, at least one instance of R¹ is optionally substituted heterocyclyl, e.g., optionally substituted 3-14 membered heterocyclyl, optionally substituted 3-10 membered heterocyclyl, optionally substituted 5-8 membered heterocyclyl, optionally substituted 5-6 membered heterocyclyl, optionally substituted 5 membered heterocyclyl, or optionally substituted 6 membered heterocyclyl.

In certain embodiments, at least one instance of R¹ is optionally substituted aryl, e.g., optionally substituted phenyl.

In certain embodiments, at least one instance of R¹ is optionally substituted heteroaryl, e.g., optionally substituted 5-14 membered heteroaryl, optionally substituted 5-10 membered heteroaryl, optionally substituted 5-6 membered heteroaryl, optionally substituted 5 membered heteroaryl, or optionally substituted 6 membered heteroaryl.

In any of the above embodiments, the R¹ alkyl, alkenyl, alkynyl, carbocyclyl, heterocyclyl, aryl, or heteroaryl group may be substituted, for example, with an optionally substituted amino group (e.g., —NR⁶R⁷), an optionally substituted hydroxyl group (e.g., —OR⁶), an optionally substituted thiol group (e.g., —SR⁶), or with a group of formula (i), (ii), or (iii), wherein each instance of R⁶ and R⁷ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or a group of formula (i), (ii), or (iii).

For example, in certain embodiments, at least one instance of R¹ is an alkyl, alkenyl, alkynyl, carbocyclyl, heterocyclyl, aryl, or heteroaryl group substituted with an amino group of the formula —N(R⁶)(R⁷). In that instance, in certain embodiments, at least one instance of R¹ is a group of formula:

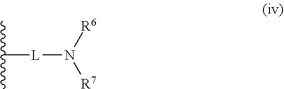

wherein:
L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, or combination thereof, and
R⁶ and R⁷ are independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group;
provided at least one instance of R⁶ and R⁷ is a group of the formula (i), (ii), or (iii):

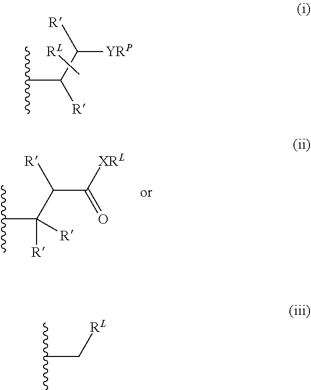

wherein R′, X, Y, R^{L}, and R^{P} are as defined herein.

In certain embodiments, at least two instances of R¹ is a group of formula (iv). In certain embodiments, at least three instances of R¹ is a group of formula (iv). In certain embodiments, at least four instances of R¹ is a group of formula (iv). In certain embodiments, at least five instances of R¹ is a group of formula (iv). In certain embodiments, at least six instances of R¹ is a group of formula (iv). In certain embodiments, at least seven instances of R¹ is a group of formula (iv). In certain embodiments, at least eight instances of R¹ is a group of formula (iv). In certain embodiments, at least nine instances of R¹ is a group of formula (iv). In certain embodiments, each instance of R¹ is a group of formula (iv).

In certain embodiments, L is an optionally substituted alkylene; e.g., optionally substituted C₁₋₅₀alkylene, optionally substituted C₁₋₄₀alkylene, optionally substituted C₁₋₃₀alkylene, optionally substituted C₁₋₂₀alkylene, optionally substituted C₄₋₂₀alkylene, optionally substituted C₆₋₂₀alkylene, optionally substituted C₈₋₂₀alkylene, optionally substituted C₁₀₋₂₀alkylene, optionally substituted C₁₋₆alkylene, optionally substituted C₂₋₆alkylene, optionally substituted C₃₋₆alkylene, optionally substituted C₄₋₆alkylene, optionally substituted C₄₋₅alkylene, or optionally substituted C₃₋₄alkylene.

In certain embodiments, L is an optionally substituted alkenylene, e.g., optionally substituted C₂₋₅₀ alkenylene, optionally substituted C₂₋₄₀alkenylene, optionally substituted C₂₋₃₀alkenylene, optionally substituted C₂₋₂₀alkenylene, optionally substituted C₄₋₂₀alkenylene, optionally substituted C₆₋₂₀alkenylene, optionally substituted C₈₋₂₀alkenylene, optionally substituted C₁₀₋₂₀alkenylene, optionally substituted C₂₋₆alkenylene, optionally substituted C₃₋₆alkenylene, optionally substituted C₄₋₆alkenylene, optionally substituted C₄₋₅alkenylene, or optionally substituted C₃₋₄alkenylene.

In certain embodiments, L is an optionally substituted alkynylene, e.g., optionally substituted C₂₋₅₀ alkynylene, optionally substituted C₂₋₄₀alkynylene, optionally substituted C₂₋₃₀alkynylene, optionally substituted C₂₋₂₀alkynylene, optionally substituted C₄₋₂₀alkynylene, optionally substituted C₆₋₂₀alkynylene, optionally substituted C₈₋₂₀alkynylene, optionally substituted C₁₀₋₂₀alkynylene, optionally substituted C₂₋₆alkynylene, optionally substituted C₃₋₆alkynylene, optionally substituted C₄₋₆alkynylene, optionally substituted C₄₋₅alkynylene, or optionally substituted C₃₋₄alkynylene.

In certain embodiments, L is an optionally substituted heteroalkylene; e.g., optionally substituted heteroC₁₋₅₀alkylene, optionally substituted heteroC₁₋₄₀alkylene, optionally substituted heteroC₁₋₃₀alkylene, optionally substituted heteroC₁₋₂₀alkylene, optionally substituted heteroC₄₋₂₀alkylene, optionally substituted heteroC₆₋₂₀alkylene, optionally substituted heteroC₈₋₂₀alkylene, optionally substituted heteroC₁₀₋₂₀alkylene, optionally substituted heteroC₁₋₆alkylene, optionally substituted heteroC₂₋₆alkylene, optionally substituted heteroC₃₋₆alkylene, optionally substituted heteroC₄₋₆alkylene, optionally substituted heteroC₄₋₅alkylene, or optionally substituted heteroC₃₋₄alkylene.

In certain embodiments, L is an optionally substituted heteroalkenylene, e.g., optionally substituted heteroC₂₋₅₀ alkenylene, optionally substituted heteroC₂₋₄₀alkenylene, optionally substituted heteroC₂₋₃₀alkenylene, optionally substituted heteroC₂₋₂₀alkenylene, optionally substituted heteroC₄₋₂₀alkenylene, optionally substituted heteroC₆₋₂₀alkenylene, optionally substituted heteroC₈₋₂₀alkenylene, optionally substituted heteroC₁₀₋₂₀alkenylene, optionally substituted heteroC₂₋₆alkenylene, optionally substituted heteroC₃₋₆alkenylene, optionally substituted heteroC₄₋₆alkenylene, optionally substituted heteroC₄₋₅alkenylene, or optionally substituted heteroC₃₋₄alkenylene.

In certain embodiments, L is an optionally substituted heteroalkynylene, e.g., optionally substituted heteroC₂₋₅₀ alkynylene, optionally substituted heteroC₂₋₄₀alkynylene, optionally substituted heteroC₂₋₃₀alkynylene, optionally substituted heteroC₂₋₂₀alkynylene, optionally substituted heteroC₄₋₂₀alkynylene, optionally substituted heteroC₆₋₂₀alkynylene, optionally substituted heteroC₈₋₂₀alkynylene, optionally substituted heteroC₁₀₋₂₀alkynylene, optionally substituted heteroC₂₋₆alkynylene, optionally substituted heteroC₃₋₆alkynylene, optionally substituted heteroC₄₋₆alkynylene, optionally substituted heteroC₄₋₅alkynylene, or optionally substituted heteroC₃₋₄alkynylene.

In certain embodiments, L is an optionally substituted carbocyclylene, e.g., optionally substituted C₃₋₁₀ carbocyclylene, optionally substituted C₅₋₈ carbocyclylene, optionally substituted C₅₋₆ carbocyclylene, optionally substituted C₅ carbocyclylene, or optionally substituted C₆ carbocyclylene.

In certain embodiments, L is an optionally substituted heterocyclylene, e.g., optionally substituted 3-14 membered heterocyclylene, optionally substituted 3-10 membered heterocyclylene, optionally substituted 5-8 membered heterocyclylene, optionally substituted 5-6 membered heterocyclylene, optionally substituted 5 membered heterocyclylene, or optionally substituted 6 membered heterocyclylene.

In certain embodiments, L is an optionally substituted arylene, e.g., optionally substituted phenylene.

In certain embodiments, L is an optionally substituted heteroarylene, e.g., optionally substituted 5-14 membered heteroarylene, optionally substituted 5-10 membered heteroarylene, optionally substituted 5-6 membered heteroarylene, optionally substituted 5 membered heteroarylene, or optionally substituted 6 membered heteroarylene.

For example, in certain embodiments, wherein L is an optionally substituted alkylene group, the group of formula (iv) is a group of the formula:

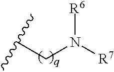

wherein q is an integer between 1 and 50, inclusive.

In certain embodiments, q is an integer between 1 and 40, inclusive. In certain embodiments, q is an integer between 1 and 30, inclusive. In certain embodiments, q is an integer between 1 and 20, inclusive. In certain embodiments, q is an integer between 4 and 20, inclusive. In certain embodiments, q is an integer between 6 and 20, inclusive. In certain embodiments, q is an integer between 8 and 20, inclusive. In certain embodiments, q is 1. In certain embodiments, q is 2. In certain embodiments, q is 3. In certain embodiments, q is 4. In certain embodiments, q is 5. In certain embodiments, q is 6. In certain embodiments, q is 7. In certain embodiments, q is 8. In certain embodiments, q is 9. In certain embodiments, q is 10.

In certain embodiments, both R⁶ and R⁷ are hydrogen. In certain embodiments, R⁶ is hydrogen and R⁷ is a group of the formula (i), (ii), or (iii). In certain embodiments, R⁶ is hydrogen and R⁷ is a group of the formula (i). In certain embodiments, R⁶ is hydrogen and R⁷ is a group of the formula (ii). In certain embodiments, R⁶ is hydrogen and R⁷ is a group of the formula (iii). In certain embodiments, both R⁶ and R⁷ are independently a group of the formula (i), (ii), or (iii). In certain embodiments, both R⁶ and R⁷ are independently a group of the formula (i). In certain embodiments, both R⁶ and R⁷ are independently a group of the formula (ii). In certain embodiments, both R⁶ and R⁷ are independently a group of the formula (iii). In certain embodiments, both R⁶ and R⁷ are the same group, selected from a group of the formula (i), (ii), or (iii).

It is understood that R¹ encompasses amino acid side chains. In certain embodiments, R¹ is a group selected from any one of the amino acid side chain groups listed therein.

In certain embodiments, each instance of R¹ is the same. In certain embodiments, at least one R¹ group is different. In certain embodiments, each R¹ group is different.

As generally defined above, each instance of R² is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii):

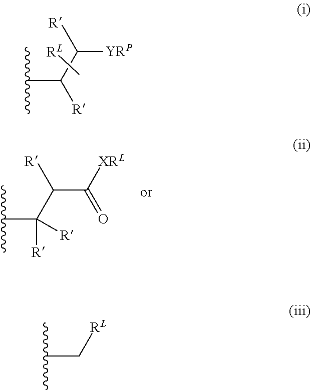

wherein R′, X, Y, R^{L}, and R^{P} are as defined herein.

In certain embodiments, at least one instance of R² is optionally substituted alkyl; e.g., optionally substituted C₁₋₆alkyl, optionally substituted C₂₋₆alkyl, optionally substituted C₃₋₆alkyl, optionally substituted C₄₋₆alkyl, optionally substituted C₄₋₅alkyl, or optionally substituted C₃₋₄alkyl.

In certain embodiments, at least one instance of R² is optionally substituted alkenyl, e.g., optionally substituted C₂₋₆alkenyl, optionally substituted C₃₋₆alkenyl, optionally substituted C₄₋₆alkenyl, optionally substituted C₄₋₅alkenyl, or optionally substituted C3-4alkenyl.

In certain embodiments, at least one instance of R² is optionally substituted alkynyl, e.g., optionally substituted C₂₋₆alkynyl, optionally substituted C₃₋₆alkynyl, optionally substituted C₄₋₆alkynyl, optionally substituted C₄₋₅alkynyl, or optionally substituted C₃₋₄alkynyl.

In certain embodiments, at least one instance of R² is optionally substituted carbocyclyl, e.g., optionally substituted C₃₋₁₀ carbocyclyl, optionally substituted C₅₋₈ carbocyclyl, optionally substituted C₅₋₆ carbocyclyl, optionally substituted C₅ carbocyclyl, or optionally substituted C₆ carbocyclyl.

In certain embodiments, at least one instance of R² is optionally substituted heterocyclyl, e.g., optionally substituted 3-14 membered heterocyclyl, optionally substituted 3-10 membered heterocyclyl, optionally substituted 5-8 membered heterocyclyl, optionally substituted 5-6 membered heterocyclyl, optionally substituted 5 membered heterocyclyl, or optionally substituted 6 membered heterocyclyl.

In certain embodiments, at least one instance of R² is optionally substituted aryl, e.g., optionally substituted phenyl.

In certain embodiments, at least one instance of R² is optionally substituted heteroaryl, e.g., optionally substituted 5-14 membered heteroaryl, optionally substituted 5-10 membered heteroaryl, optionally substituted 5-6 membered heteroaryl, optionally substituted 5 membered heteroaryl, or optionally substituted 6 membered heteroaryl.

In certain embodiments, at least one instance of R² is a nitrogen protecting group.

In certain embodiments, at least one instance of R² is a group of the formula (i). In certain embodiments, at least one instance of R² is a group of the formula (ii). In certain embodiments, at least one instance of R² is a group of the formula (iii).

In certain embodiments, each instance of R² is a group other than formula (i), (ii), or (iii); in that instance, it follows that at least one R^{Q} is a group of the formula (i), (ii), or (iii), or at least one R¹ is a group of formula (iv), and at least one of R⁶ or R⁷ encompassed by R¹ is a group of the formula (i), (ii), or (iii). For example, in certain embodiments, both instances of R² are hydrogen, and thus at least one R^{Q} is a group of the formula (i), (ii), or (iii), or at least one R¹ is a group of formula (iv), and at least one of R⁶ or R⁷ encompassed by R¹ is a group of the formula (i), (ii), or (iii).

As understood from the above discussion, compounds of Formula (I) each include at least one instance of a group of the formula (i), (ii), or (iii):

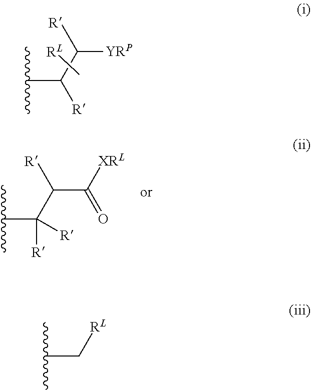

wherein:
each instance of R′ is independently hydrogen or optionally substituted alkyl;
X is O, S, NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
Y is O, S, NR^{Y}, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and
R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer.

In certain embodiments, a compound of Formula (I) comprises at least one instance of a group of the formula (i) attached thereto:

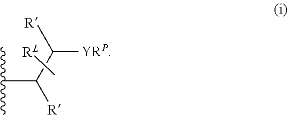

In certain embodiments of formula (i), Y is O. In certain embodiments of formula (i), Y is S. In certain embodiments of formula (i), Y is NR^{Y}, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments of formula (i), Y is NR^{Y}, wherein R^{Y} is hydrogen, optionally substituted alkyl, or a nitrogen protecting group. In certain embodiments of formula (i), each instance of R′ is hydrogen.

As used herein, when the group R^{L} is depicted as bisecting a carbon-carbon bond, e.g., of the group of the formula (i), it is understood that R^{L} may be substituted at either carbon. Nucleophilic attack of an amino or amide group at the least sterically hindered carbon of the epoxide, thiirane, or aziridine of formula (i-x) provides a group of the formula (i-a1), (i-a2), or (i-a3) (route a), while nucleophilic attack at the more sterically hindered carbon of the epoxide, thiirane, or aziridine of formula (i-x) provides a group of the formula (i-b1), (i-b2), or (i-b3) (route b), wherein R is hydrogen (Scheme 1). It is understood that compounds of the present invention may comprise a mixture of products attached thereto arising from route (a) and route (b) depending on the preference, or lack thereof, of the mode of addition. The bisecting group R^{L} depicted in the Formulae seeks to encompasses all contemplated modes of addition.

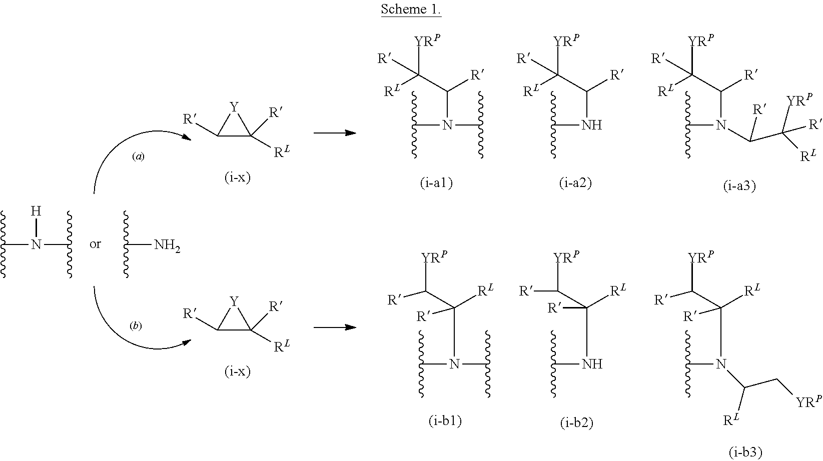

The resulting hydroxyl, thiol, or amino group —YR^{P}, wherein R^{P} is hydrogen, may optionally be converted to a substituted group, wherein R^{P} is a group other than hydrogen, i.e., wherein R^{P} is independently selected from optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; using conventional methods. Alkylation, acylation, and/or protection of a hydroxyl, thiol, or amino moiety are methods well-known in the art; see, e.g., Protecting Groups in Organic Synthesis, T. W. Greene and P. G. M. Wuts, 3^{rd} edition, John Wiley & Sons, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. For example, in certain non-limiting embodiments, the hydroxyl, thiol, or amino moiety —YR^{P}, wherein R^{P} is hydrogen, may be reacted with an electrophile of the formula R^{P}—X² wherein R^{P} is a group other than hydrogen, and X² is a leaving group, to provide a substituted hydroxyl, thiol, and amino group in formula (i).

In certain embodiments of formula (i), R^{P} is hydrogen. In certain embodiments of formula (i), R^{P} is optionally substituted alkyl. In certain embodiments of formula (i), R^{P} is optionally substituted alkenyl. In certain embodiments of formula (i), R^{P} is optionally substituted alkynyl. In certain embodiments of formula (i), R^{P} is optionally substituted carbocyclyl. In certain embodiments of formula (i), R^{P} is optionally substituted heterocyclyl. In certain embodiments of formula (i), R^{P} is optionally substituted aryl. In certain embodiments of formula (i), R^{P} is optionally substituted heteroaryl. In certain embodiments of formula (i), R^{P} is an oxygen protecting group when attached to an oxygen atom. In certain embodiments of formula (i), R^{P} is a sulfur protecting group when attached to a sulfur atom. In certain embodiments of formula (i), R^{P} is a nitrogen protecting group when attached to a nitrogen atom.

It is understood from the present disclosure that the group of formula (i) represents a group of formula (i-a) or a group of formula (i-b):

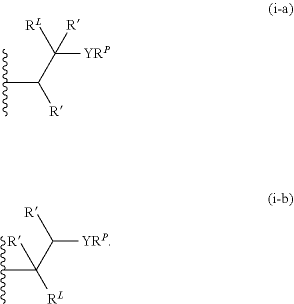

In certain embodiments, the reaction mixture provides a mixture of compounds of Formula (I) comprising more conjugate to a group of formula (i-a) than to formula (i-b), e.g., the reaction mixture comprises greater than 50%, greater than 60%, greater than 70%, greater than 80%, greater than 90%, greater than 95%, greater than 99%, between about 60% to about 100%, between about 70% to about 100%, between about 80% to about 100%, between about 90% to about 100%, between about 95% to about 100%, or between about 99% to about 100%, attached to formula (i-a).

In certain embodiments, the epoxide, thiirane, or aziridine of formula (i-x) is chiral, i.e., having (R) or (S) stereochemistry. Chiral epoxides, thiiranes, and aziridines can be obtained from a variety of sources which are familiar to those skilled in the art of organic synthesis. In some embodiments, the chiral epoxide, thiirane, or aziridine is obtained commercially. In some embodiments, the chiral epoxide, thiirane, or aziridine is synthesized according to methods known to those of skill in the art, such as, but not limited to the Sharpless epoxidation of primary and secondary allylic alcohols into 2,3-epoxyalcohols (see, e.g., Katsuki et al., J. Am. Chem. Soc. 1980, 102, 5974; Hill et al., Org. Syn., Coll. Vol. 7, p. 461 (1990); Vol. 63, p. 66 (1985); Katsuki et al., Org. React. 1996, 48, 1-300). In some embodiments, the chiral epoxide, thiirane, or aziridine is obtained from the resolution of a mixture (e.g., racemic mixture) of epoxides, thiiranes, or aziridines. In some embodiments, the chiral epoxide, thiirane, or aziridine is obtained by the separation of enantiomers or diastereoisomers using chiral chromatography. Chirality can be characterized in a variety of ways, e.g., obtaining a crystal structure of the compound containing a heavy atom attached thereto, obtaining the optical rotation of the compound, and/or NMR analysis after chemical modification of the optically active compound with a chiral derivatizing agent are some methods useful in evaluating chirality.

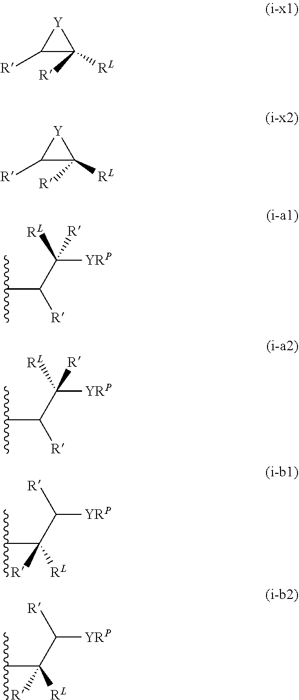

In certain embodiments, wherein the epoxide, thiirane, or aziridine of formula (i-x1) is chiral, the conjugation reaction is regioselective, and the reaction provides a chiral mixture of compounds of Formula (I) comprising more conjugated to a group of formula (i-a1) than formula (i-b1), e.g., the reaction mixture comprises greater than 50%, greater than 60%, greater than 70%, greater than 80%, greater than 90%, greater than 95%, greater than 99%, between about 60% to about 100%, between about 70% to about 100%, between about 80% to about 100%, between about 90% to about 100%, between about 95% to about 100%, or between about 99% to about 100% attached to formula (i-a1).

In other embodiments, wherein the epoxide, thiirane, or aziridine of formula (i-x2) is chiral, the conjugation reaction is regioselective, and the reaction provides a chiral mixture of compounds of Formula (I) comprising more conjugated to a group of formula (i-a2) than formula (i-b2), e.g., the reaction mixture comprises greater than 50%, greater than 60%, greater than 70%, greater than 80%, greater than 90%, greater than 95%, greater than 99%, between about 60% to about 100%, between about 70% to about 100%, between about 80% to about 100%, between about 90% to about 100%, between about 95% to about 100%, or between about 99% to about 100% attached to formula (i-a2).

In certain embodiments, a compound of Formula (I) comprises at least one instance of a group of the formula (ii) attached thereto:

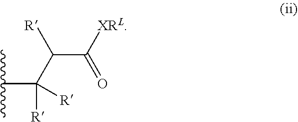

In certain embodiments of formula (ii), X is O. In certain embodiments of formula (ii), X is S. In certain embodiments of formula (ii), X is NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments of formula (ii), X is NR^{X}, wherein R^{X} is hydrogen, optionally substituted alkyl, or a nitrogen protecting group. In certain embodiments of formula (i), each instance of R′ is hydrogen.

In certain embodiments, a compound of Formula (I) comprises at least one instance of a group of the formula (ii) attached thereto:

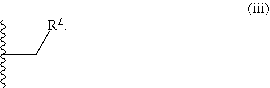

As generally defined above, R^{L} is optionally substituted C₁₋₅₀ alkyl, optionally substituted C₂₋₅₀ alkenyl, optionally substituted C₂₋₅₀ alkynyl, optionally substituted C₁₋₅₀ heteroalkyl, optionally substituted C₂₋₅₀ heteroalkenyl, optionally substituted C₂₋₅₀ heteroalkynyl, or a polymer. The group R^{L} seeks to encompass lipophilic, hydrophobic, and/or non-polar groups, but such terms should not limit the scope of R^{L}.

In certain embodiments, at least one instance of R^{L} is an optionally substituted C₁₋₅₀ alkyl. In certain embodiments, R^{L} is an optionally substituted C₆₋₅₀alkyl. In certain embodiments, R^{L} is an optionally substituted C₆₋₄₀alkyl. In certain embodiments, R^{L} is an optionally substituted C₆₋₃₀alkyl. In certain embodiments, R^{L} is an optionally substituted C₆₋₂₀alkyl. In certain embodiments, R^{L} is an optionally substituted C₈₋₂₀alkyl. In certain embodiments, R^{L} is an optionally substituted C₈alkyl. In certain embodiments, R^{L} is an optionally substituted C₉alkyl. In certain embodiments, R^{L} is an optionally substituted C₁₀alkyl. In certain embodiments, R^{L} is an optionally substituted C₁₁alkyl. In certain embodiments, R^{L} is an optionally substituted C₁₂alkyl. In certain embodiments, R^{L} is an optionally substituted C₁₃alkyl. In certain embodiments, R^{L} is an optionally substituted C₁₄alkyl. In certain embodiments, R^{L} is an optionally substituted C₁₅alkyl. In certain embodiments, R^{L} is an optionally substituted C₁₆alkyl. In certain embodiments, R^{L} is an optionally substituted C₁₇alkyl. In certain embodiments, R^{L} is an optionally substituted C₁₈alkyl. In certain embodiments, R^{L} is an optionally substituted C₁₉alkyl. In certain embodiments, R^{L} is an optionally substituted C₂₀alkyl. In any of the above embodiments, the group R^{L} is an unsubstituted alkyl group.

In certain embodiments, at least one instance of R^{L} is an unsubstituted alkyl. Exemplary unsubstituted alkyl groups include, but are not limited to, —CH₃, —C₂H₅, —C₃H₇, —C₄H₉, —C₅H₁₁, —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, —C₂₀H₄₁, —C₂₁H₄₃, —C₂₂H₄₅, —C₂₃H₄₇, —C₂₄H₄₉, and —C₂₅H₅₁.

In certain embodiments, at least one instance of R^{L} is a substituted alkyl. For example, in certain embodiments, at least one instance of R^{L} is an alkyl substituted with one or more fluorine substituents. Exemplary fluorinated alkyl groups include, but are not limited to:

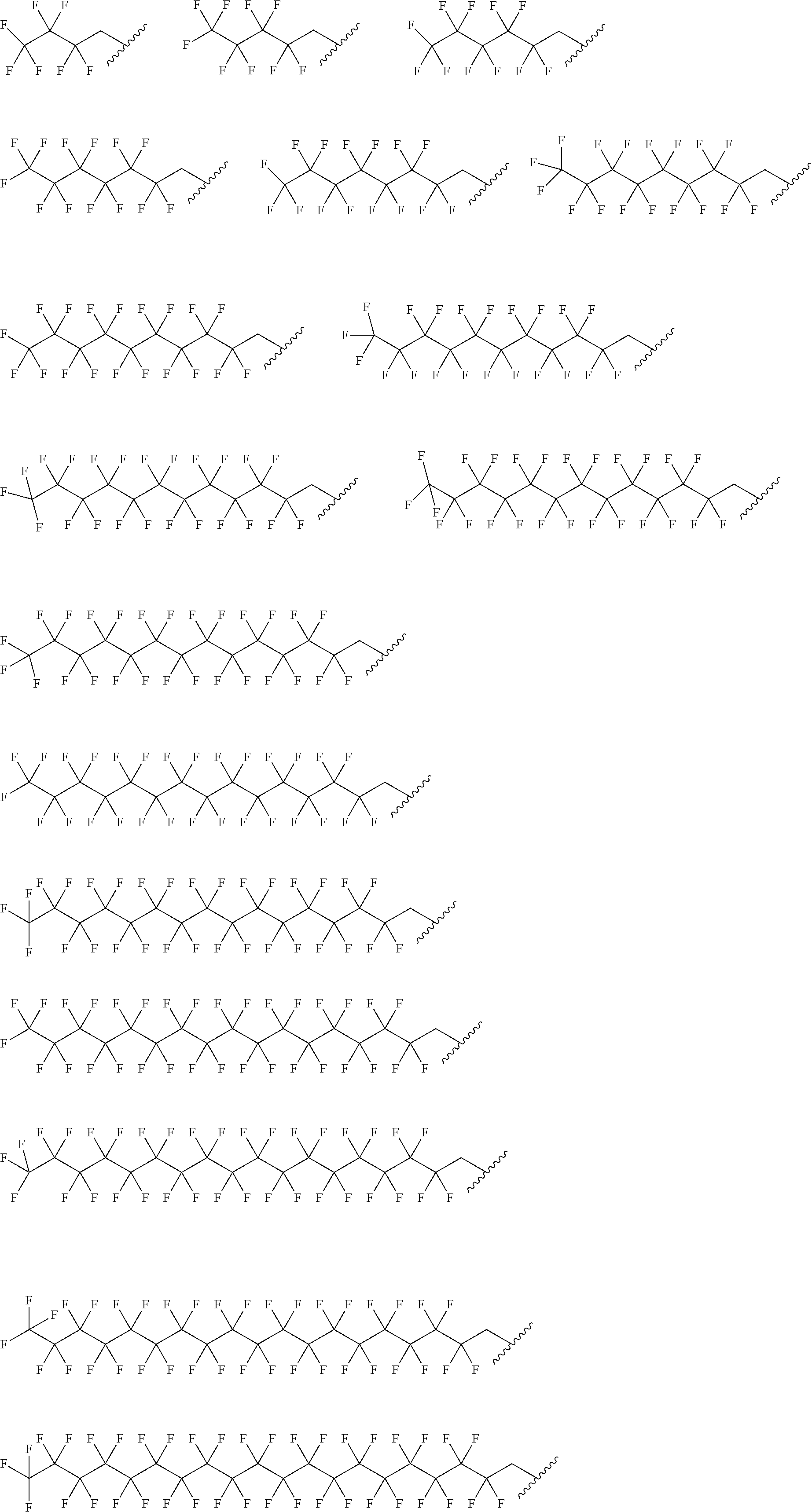

In certain embodiments, at least one instance of R^{L} is an optionally substituted C₂₋₅₀ alkenyl. In certain embodiments, R^{L} is an optionally substituted C₆₋₅₀alkenyl. In certain embodiments, R^{L} is an optionally substituted C₆₋₄₀alkenyl. In certain embodiments, R^{L} is an optionally substituted C₆₋₃₀alkenyl. In certain embodiments, R^{L} is an optionally substituted C₆₋₂₀alkenyl. In certain embodiments, R^{L} is an optionally substituted C₈₋₂₀alkenyl. In certain embodiments, R^{L} is an optionally substituted C₈alkenyl. In certain embodiments, R^{L} is an optionally substituted C₉alkenyl. In certain embodiments, R^{L} is an optionally substituted C₁₀alkenyl. In certain embodiments, R^{L} is an optionally substituted C₁₁alkenyl. In certain embodiments, R^{L} is an optionally substituted C₁₂alkenyl. In certain embodiments, R^{L} is an optionally substituted C₁₃alkenyl. In certain embodiments, R^{L} is an optionally substituted C₁₄alkenyl. In certain embodiments, R^{L} is an optionally substituted C₁₅alkenyl. In certain embodiments, R^{L} is an optionally substituted C₁₆alkenyl. In certain embodiments, R^{L} is an optionally substituted C₁₇alkenyl. In certain embodiments, R^{L} is an optionally substituted C₁₈alkenyl. In certain embodiments, R^{L} is an optionally substituted C₁₉alkenyl. In certain embodiments, R^{L} is an optionally substituted C₂₀alkenyl. In any of the above embodiments, the group R^{L} is an unsubstituted alkenyl group.

Exemplary unsubstituted alkenyl groups include, but are not limited to:

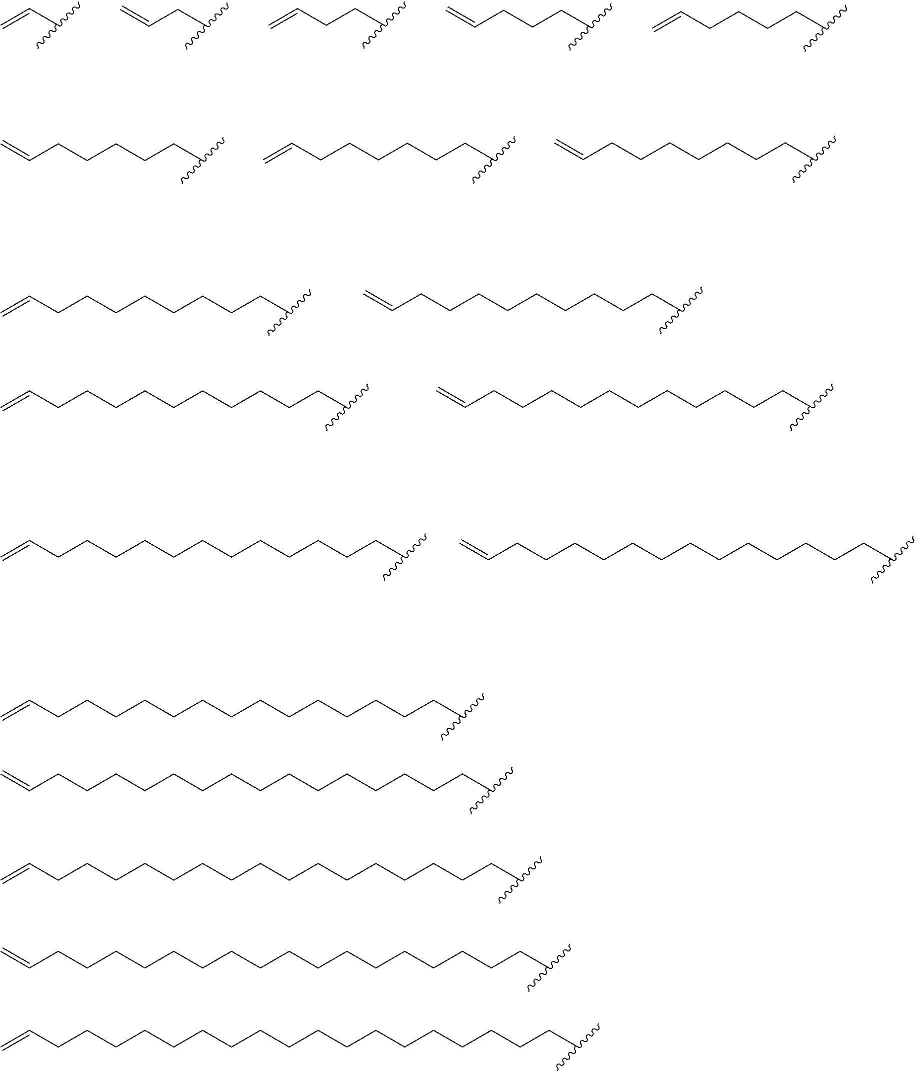

Myristoleic —(CH₂)₇CH═CH(CH₂)₃CH₃,
Palmitoliec —(CH₂)₇CH═CH(CH₂)₅CH₃,
Sapienic —(CH₂)₄CH═CH(CH₂)₈CH₃,
Oleic —(CH₂)₇CH═CH(CH₂)₇CH₃,
Linoleic —(CH₂)₇CH═CHCH₂CH═CH(CH₂)₄CH₃,
α-Linolenic —(CH₂)₇CH═CHCH₂CH═CHCH₂CH═CHCH₂CH₃,
Arachinodonic —(CH₂)₃CH═CHCH₂CH═CHCH₂CH═CHCH₂CH═CH(CH₂)₄CH₃,
Eicosapentaenoic —(CH₂)₃CH═CHCH₂CH═CHCH₂CH═CHCH₂CH═CHCH₂CH═CHCH₂CH₃,
Erucic —(CH₂)₁₁CH═CH(CH₂)₇CH₃, and
Docosahexaenoic —(CH₂)₂CH═CHCH₂CH═CHCH₂CH═CHCH₂CH═CHCH₂CH═CHCH₂CH
═CH—CH₂CH₃.

In embodiments, wherein R^{L} is defined as a C₆₋₅₀alkyl or C₆₋₅₀alkenyl groups, such groups are meant to encompass lipophilic groups (also referred to as a “lipid tail”). Lipophilic groups comprise a group of molecules that include fats, waxes, oils, fatty acids, and the like. Lipid tails present in these lipid groups can be saturated and unsaturated, depending on whether or not the lipid tail comprises double bonds. The lipid tail can also comprise different lengths, often categorized as medium (i.e., with tails between 7-12 carbons, e.g., C₇₋₁₂ alkyl or C₇₋₁₂ alkenyl), long (i.e., with tails greater than 12 carbons and up to 22 carbons, e.g., C₁₃₋₂₂ alkyl or C₁₃₋₂₂ alkenyl), or very long (i.e., with tails greater than 22 carbons, e.g., C₂₃₋₃₀ alkyl or C₂₃₋₃₀ alkenyl).

In certain embodiments, R^{L} is an optionally substituted C₂₋₅₀ alkynyl. In certain embodiments, R^{L} is an optionally substituted C₆₋₅₀alkynyl. In certain embodiments, R^{L} is an optionally substituted C₆₋₄₀alkynyl. In certain embodiments, R^{L} is an optionally substituted C₆₋₃₀alkynyl. In certain embodiments, R^{L} is an optionally substituted C₆₋₂₀alkynyl. In certain embodiments, R^{L} is an optionally substituted C₈₋₂₀alkynyl. In certain embodiments, R^{L} is an optionally substituted C₈alkynyl. In certain embodiments, R^{L} is an optionally substituted C₉alkynyl. In certain embodiments, R^{L} is an optionally substituted C₁₀alkynyl. In certain embodiments, R^{L} is an optionally substituted C₁₁alkynyl. In certain embodiments, R^{L} is an optionally substituted C₁₂alkynyl. In certain embodiments, R^{L} is an optionally substituted C₁₃alkynyl. In certain embodiments, R^{L} is an optionally substituted C₁₄alkynyl. In certain embodiments, R^{L} is an optionally substituted C₁₅alkynyl. In certain embodiments, R^{L} is an optionally substituted C₁₆alkynyl. In certain embodiments, R^{L} is an optionally substituted C₁₇alkynyl. In certain embodiments, R^{L} is an optionally substituted C₁₈alkynyl. In certain embodiments, R^{L} is an optionally substituted C₁₉alkynyl. In certain embodiments, R^{L} is an optionally substituted C20alkynyl. In any of the above embodiments, the group R^{L} is an unsubstituted alkynyl group.

In certain embodiments, at least one instance of R^{L} is an optionally substituted heteroC₁₋₅₀ alkyl. In certain embodiments, R^{L} is an optionally substituted heteroC₆₋₅₀ alkyl. In certain embodiments, R^{L} is an optionally substituted heteroC₆₋₄₀ alkyl. In certain embodiments, R^{L} is an optionally substituted heteroC₆₋₃₀alkyl. In certain embodiments, R^{L} is an optionally substituted heteroC₆₋₂₀alkyl. In certain embodiments, R^{L} is an optionally substituted heteroC₁₀₋₂₀alkyl. In certain embodiments, R^{L} is an optionally substituted heteroC₈alkyl. In certain embodiments, R^{L} is an optionally substituted heteroC₉alkyl. In certain embodiments, R^{L} is an optionally substituted heteroC₁₀alkyl. In certain embodiments, R^{L} is an optionally substituted heteroC₁₁alkyl. In certain embodiments, R^{L} is an optionally substituted heteroC₁₂alkyl. In certain embodiments, R^{L} is an optionally substituted heteroC₁₃alkyl. In certain embodiments, R^{L} is an optionally substituted heteroC₁₄alkyl. In certain embodiments, R^{L} is an optionally substituted heteroC₁₅alkyl. In certain embodiments, R^{L} is an optionally substituted heteroC₁₆alkyl. In certain embodiments, R^{L} is an optionally substituted heteroC₁₇alkyl. In certain embodiments, R^{L} is an optionally substituted heteroC₁₈alkyl. In certain embodiments, R^{L} is an optionally substituted heteroC₁₉alkyl. In certain embodiments, R^{L} is an optionally substituted heteroC₂₀alkyl. In any of the above embodiments, the group R^{L} is an unsubstituted heteroalkyl group.

Exemplary unsubstituted heteroalkyl groups include, but are not limited to,

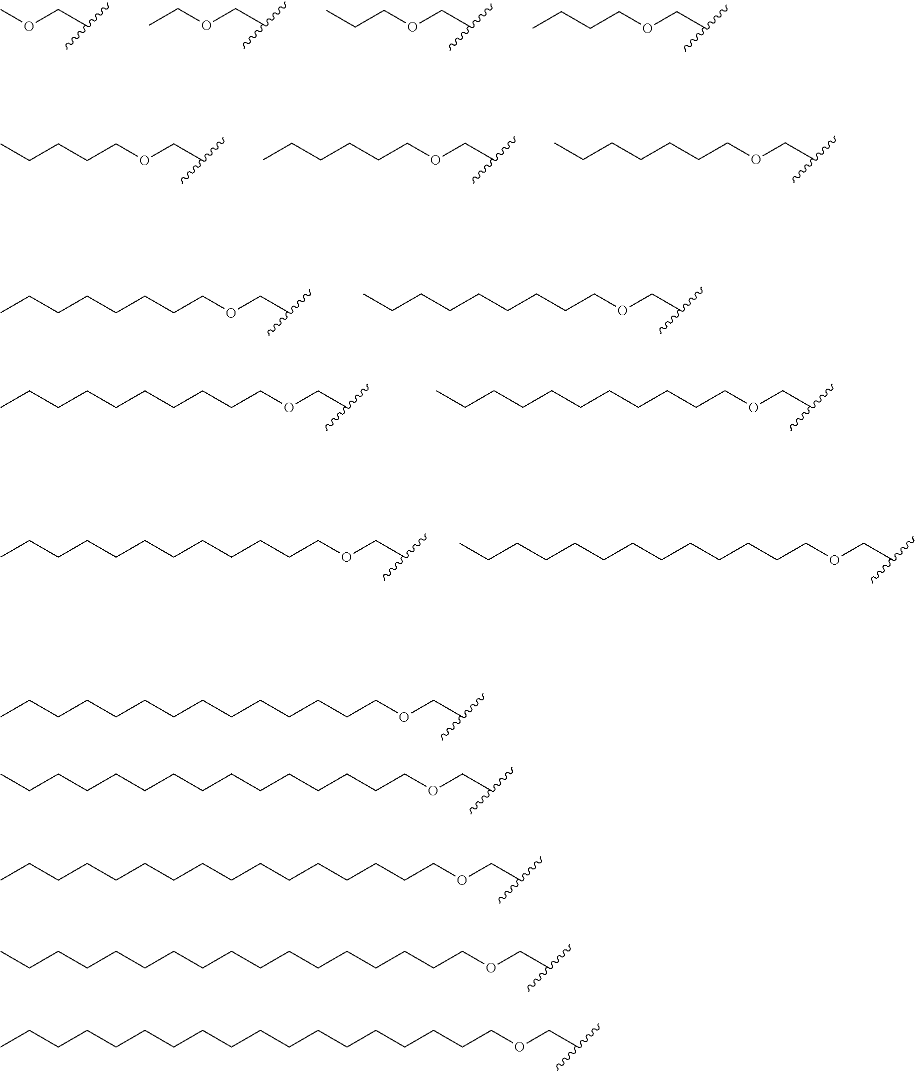

In certain embodiments, at least one instance of R^{L} is an optionally substituted heteroC₂₋₅₀ alkenyl. In certain embodiments, R^{L} is an optionally substituted heteroC₆₋₅₀alkenyl. In certain embodiments, R^{L} is an optionally substituted heteroC₆₋₄₀alkenyl. In certain embodiments, R^{L} is an optionally substituted heteroC₆₋₃₀alkenyl. In certain embodiments, R^{L} is an optionally substituted heteroC₆₋₂₀alkenyl. In certain embodiments, R^{L} is an optionally substituted heteroC₈₋₂₀alkenyl. In certain embodiments, R^{L} is an optionally substituted heteroC₈alkenyl. In certain embodiments, R^{L} is an optionally substituted heteroC₉alkenyl. In certain embodiments, R^{L} is an optionally substituted heteroC₁₀alkenyl. In certain embodiments, R^{L} is an optionally substituted heteroC₁₁alkenyl. In certain embodiments, R^{L} is an optionally substituted heteroC₁₂alkenyl. In certain embodiments, R^{L} is an optionally substituted heteroC₁₃alkenyl. In certain embodiments, R^{L} is an optionally substituted heteroC₁₄alkenyl. In certain embodiments, R^{L} is an optionally substituted heteroC₁₅alkenyl. In certain embodiments, R^{L} is an optionally substituted heteroC₁₆alkenyl. In certain embodiments, R^{L} is an optionally substituted heteroC₁₇alkenyl. In certain embodiments, R^{L} is an optionally substituted heteroC₁₈alkenyl. In certain embodiments, R^{L} is an optionally substituted heteroC₁₉alkenyl. In certain embodiments, R^{L} is an optionally substituted heteroC₂₀alkenyl. In any of the above embodiments, the group R^{L} is an unsubstituted heteroalkenyl group.

In certain embodiments, R^{L} is an optionally substituted heteroC₂₋₅₀alkynyl. In certain embodiments, R^{L} is an optionally substituted heteroC₆₋₅₀alkynyl. In certain embodiments, R^{L} is an optionally substituted heteroC₆₋₄₀alkynyl. In certain embodiments, R^{L} is an optionally substituted heteroC₆₋₃₀alkynyl. In certain embodiments, R^{L} is an optionally substituted heteroC₆₋₂₀alkynyl. In certain embodiments, R^{L} is an optionally substituted heteroC₈₋₂₀alkynyl. In certain embodiments, R^{L} is an optionally substituted heteroC₈alkynyl. In certain embodiments, R^{L} is an optionally substituted heteroC₉alkynyl. In certain embodiments, R^{L} is an optionally substituted heteroC₁₀alkynyl. In certain embodiments, R^{L} is an optionally substituted heteroC₁₁alkynyl. In certain embodiments, R^{L} is an optionally substituted heteroC₁₂alkynyl. In certain embodiments, R^{L} is an optionally substituted heteroC₁₃alkynyl. In certain embodiments, R^{L} is an optionally substituted heteroC₁₄alkynyl. In certain embodiments, R^{L} is an optionally substituted heteroC₁₅alkynyl. In certain embodiments, R^{L} is an optionally substituted heteroC₁₆alkynyl. In certain embodiments, R^{L} is an optionally substituted heteroC₁₇alkynyl. In certain embodiments, R^{L} is an optionally substituted heteroC₁₈alkynyl. In certain embodiments, R^{L} is an optionally substituted heteroC₁₉alkynyl. In certain embodiments, R^{L} is an optionally substituted heteroC₂₀alkynyl. In any of the above embodiments, the group R^{L} is an unsubstituted heteroalkynyl group.

In certain embodiments, at least one instance of R^{L} is a polymer. As used herein, a “polymer” refers to a compound comprised of at least 3 (e.g., at least 10, 20, 30, 40, 50, 60, 70, 80, 90, 100, etc.) repeating covalently bound structural units. The polymer is in certain embodiments biocompatible (i.e., non-toxic). Exemplary polymers include, but are not limited to, cellulose polymers (e.g., hydroxyethylcellulose, ethylcellulose, carboxymethylcellulose, methyl cellulose, hydroxypropylmethylcellulose (HPMC)), dextran polymers, polymaleic acid polymers, poly(acrylic acid) polymers, poly(vinylalcohol) polymers, polyvinylpyrrolidone (PVP) polymers, and polyethyleneglycol (PEG) polymers, and combinations thereof.

In certain embodiments, the ionizable amino lipid is selected from the group consisting of:

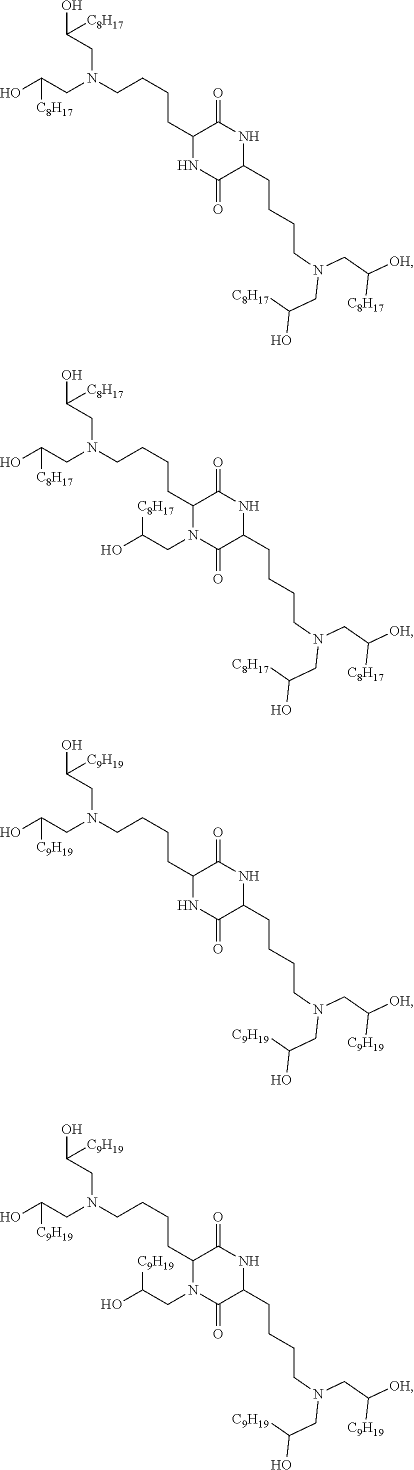

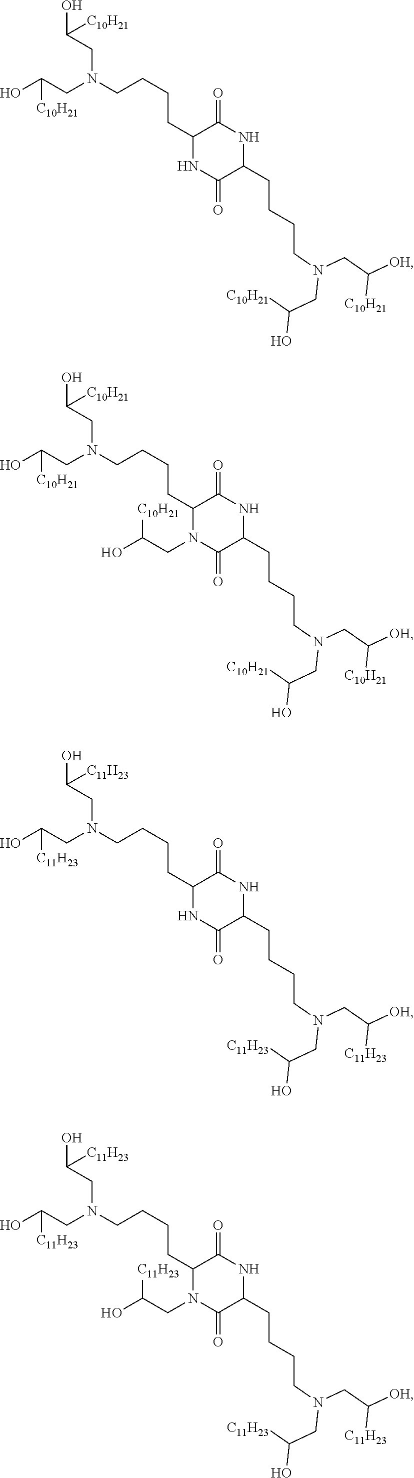

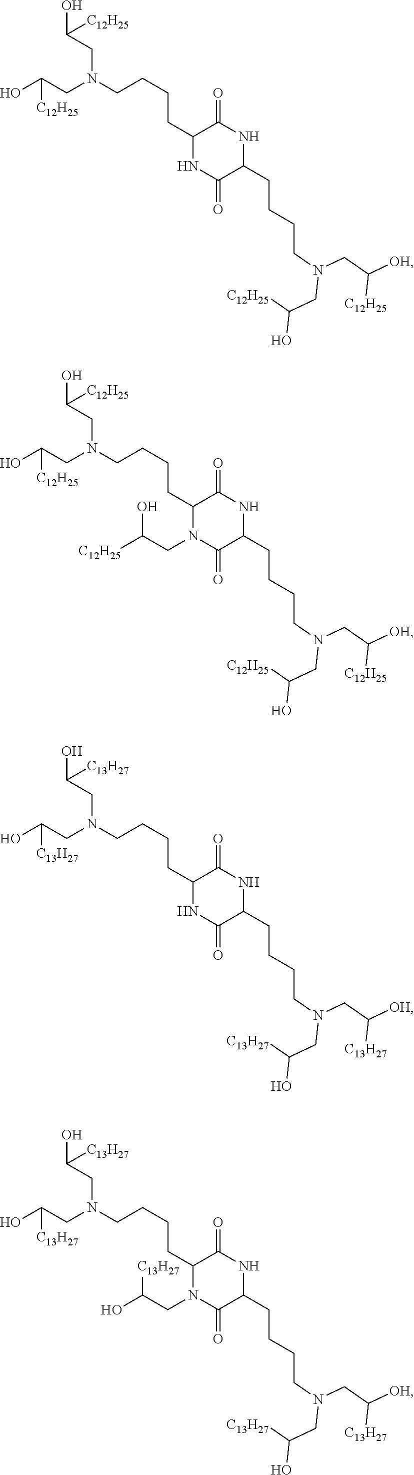

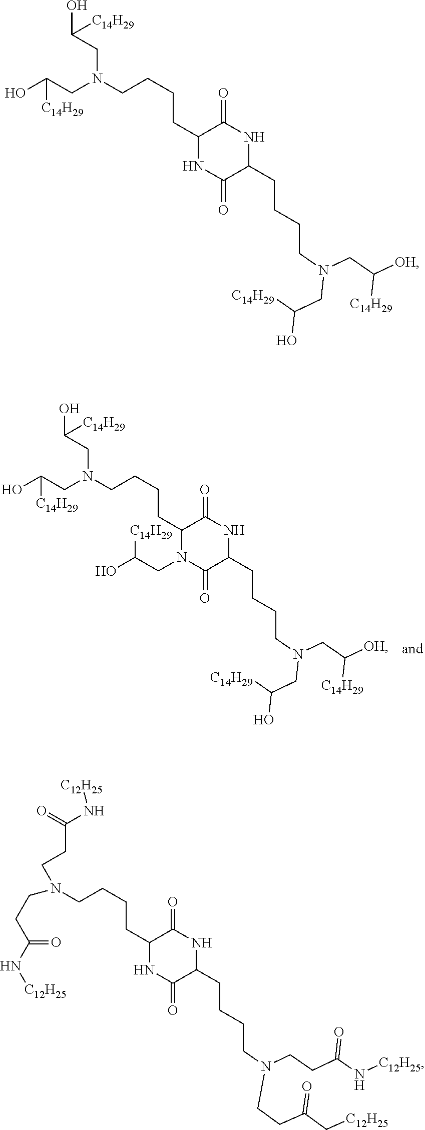

and salts thereof.

### Formula (II)

In certain embodiments, the ionizable amino lipid is a compound of Formula (II):

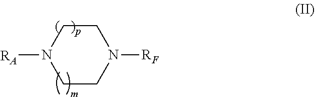

wherein:
p is an integer between 1 and 3, inclusive;
m is an integer between 1 and 3, inclusive;
R_{A} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

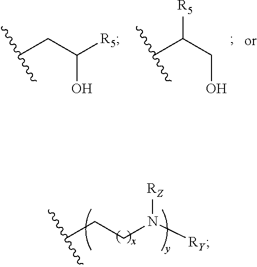

R_{F} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

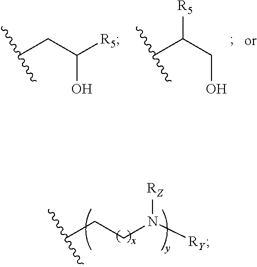

each occurrence of R₅ is independently hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; or substituted or unsubstituted heteroaryl;
wherein, at least one of R_{A}, R_{F}, R_{Y}, and R_{Z} is

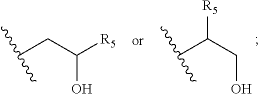

each occurrence of x is an integer between 1 and 10, inclusive;
each occurrence of y is an integer between 1 and 10, inclusive;
each occurrence of R_{Y} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

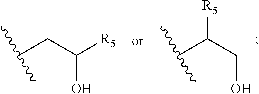

each occurrence of R_{Z} is hydrogen; substituted or unsubstituted, cyclic or acyclic, branched or unbranched C₁₋₂₀ aliphatic; substituted or unsubstituted, cyclic or acyclic, branched or unbranched C₁₋₂₀ heteroaliphatic; substituted or unsubstituted aryl; substituted or unsubstituted heteroaryl;

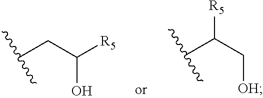

or a pharmaceutically acceptable salt thereof.

The following chemical group definitions and embodiments apply to compounds of Formula (II) and all subgenera thereof provided herein.

In certain embodiments, R_{A} is hydrogen. In certain embodiments, R_{A} is hydrogen. In certain embodiments, R_{A} is substituted or unsubstituted, cyclic or acyclic, branched or unbranched C₁₋₂₀ aliphatic. In certain embodiments, R_{A} is C₁-C₆ aliphatic. In certain embodiments, R_{A} is C₁-C₆ alkyl. In certain embodiments, R_{A} is substituted or unsubstituted, cyclic or acyclic, branched or unbranched C₁₋₂₀ heteroaliphatic. In certain embodiments, R_{A} is substituted or unsubstituted aryl. In certain embodiments, R_{A} is substituted or unsubstituted heteroaryl. In certain embodiments, R_{A} is

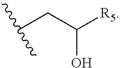

In certain embodiments, R_{A} is

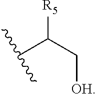

In certain embodiments, R_{A} is

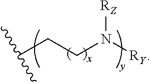

In certain embodiments R_{A} is

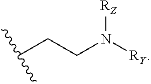

In certain embodiments R_{A} is

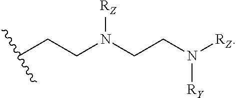

In certain embodiments R_{A} is

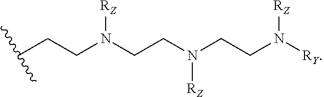

In certain embodiments, R_{F} is hydrogen. In certain embodiments, no R_{F} is hydrogen. In certain embodiments, R_{F} is substituted or unsubstituted, cyclic or acyclic, branched or unbranched C₁₋₂₀ aliphatic. In certain embodiments, R_{F} is C₁-C₆ aliphatic. In certain embodiments, R_{F} is C₁-C₆ alkyl. In certain embodiments, R_{F} is substituted or unsubstituted, cyclic or acyclic, branched or unbranched C₁₋₂₀ heteroaliphatic. In certain embodiments, R_{F} is substituted or unsubstituted aryl. In certain embodiments, R_{F} is substituted or unsubstituted heteroaryl. In certain embodiments, R_{F} is

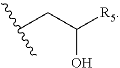

In certain embodiments, R_{F} is

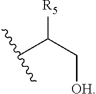

In certain embodiments, R_{F} is

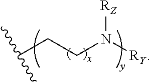

In certain embodiments R_{F} is

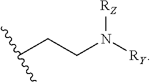

In certain embodiments R_{F} is

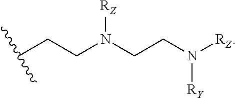

In certain embodiments R_{F} is

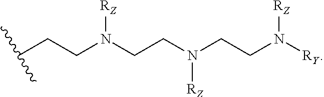

In certain embodiments R_{F} is

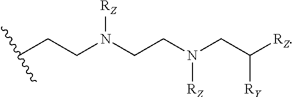

In certain embodiments R_{F} is

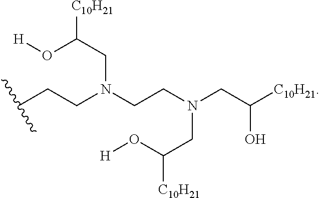

In certain embodiments, no R_{A} is hydrogen and no R_{F} is hydrogen. In certain embodiments, R_{A} is

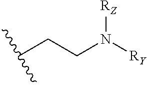

### and R_{F} is

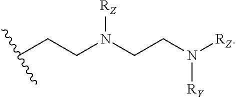

In certain embodiments, R_{A} is

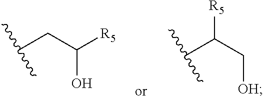

### and R_{F} is

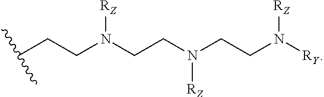

In certain embodiments, R_{A} is

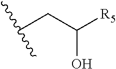

### and R_{F} is

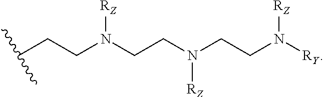

In certain embodiments, R_{A} is

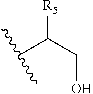

### and R_{F} is

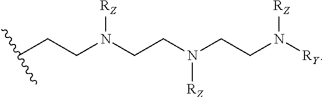

In certain embodiments, R_{A} is

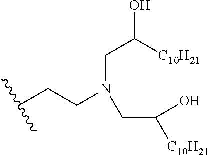

### and R_{F} is

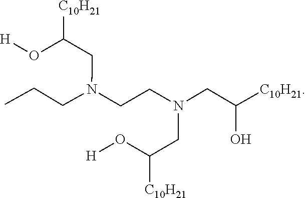

In certain embodiments, m is 1. In certain embodiments, m is 2. In certain embodiments, m is 3.

In certain embodiments, p is 1. In certain embodiments, p is 2. In certain embodiments, p is 3.

In certain embodiments, R₅ is hydrogen. In certain embodiments, R₅ is substituted or unsubstituted, cyclic or acyclic, branched or unbranched C₁₋₂₀ aliphatic. In certain embodiments, R₅ is C₈-C₁₆ aliphatic. In certain embodiments, R₅ is C₈-C₁₆ alkyl. In some embodiments, R₅ is an unsubstituted and unbranched, C₁₀₋₁₂-aliphatic group. In some embodiments, R₅ is

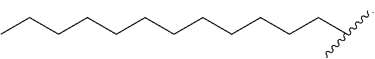 [SMILES: *CCCCCCCCCCCC]

In some embodiments, R₅ is

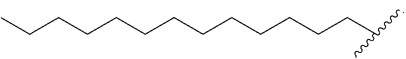 [SMILES: *CCCCCCCCCCCCC]

In some embodiments, R₅ is

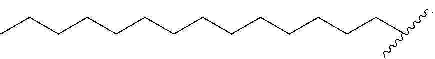 [SMILES: *CCCCCCCCCCCCCC]

In certain embodiments, R₅ is selected from the following formulae:

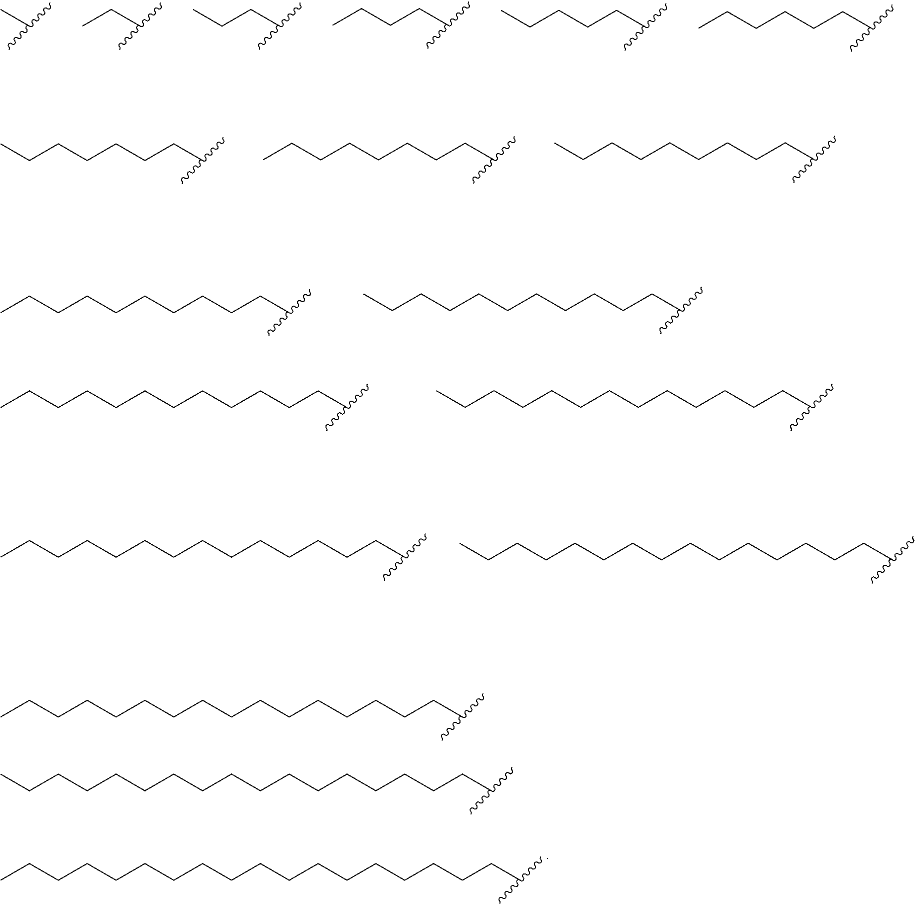

In certain embodiments, R₅ is a C₁₋₂₀ alkenyl moiety, optionally substituted. In certain embodiments, R₅ is selected from the following formulae:

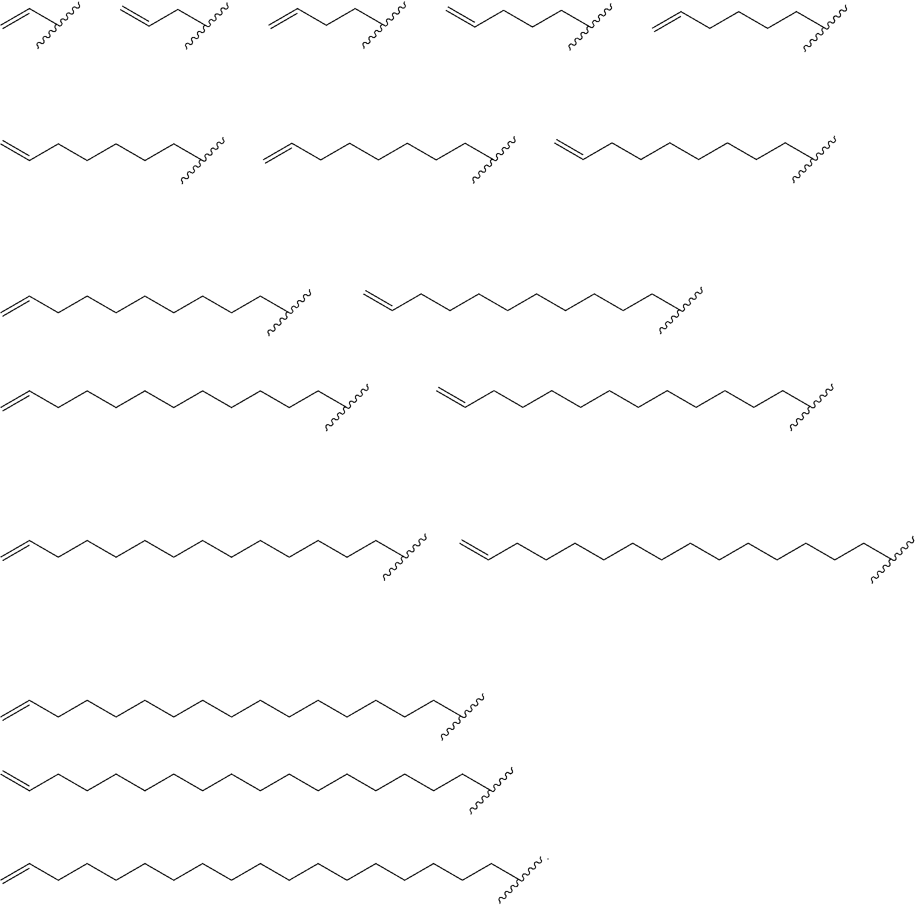

In certain embodiments, R₅ is substituted or unsubstituted, cyclic or acyclic, branched or unbranched C₁₋₂₀ heteroaliphatic. In some embodiments, R₅ is an unsubstituted and unbranched, C₁₃ heteroaliphatic group. In some embodiments, R₅ is an unsubstituted and unbranched, C₁₄ heteroaliphatic group. In certain embodiments, R₅ is:

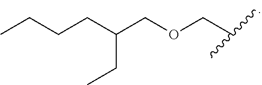 [SMILES: *COCC(CC)CCCC]

In certain embodiments, R₅ is, independently, selected from the following formulae:

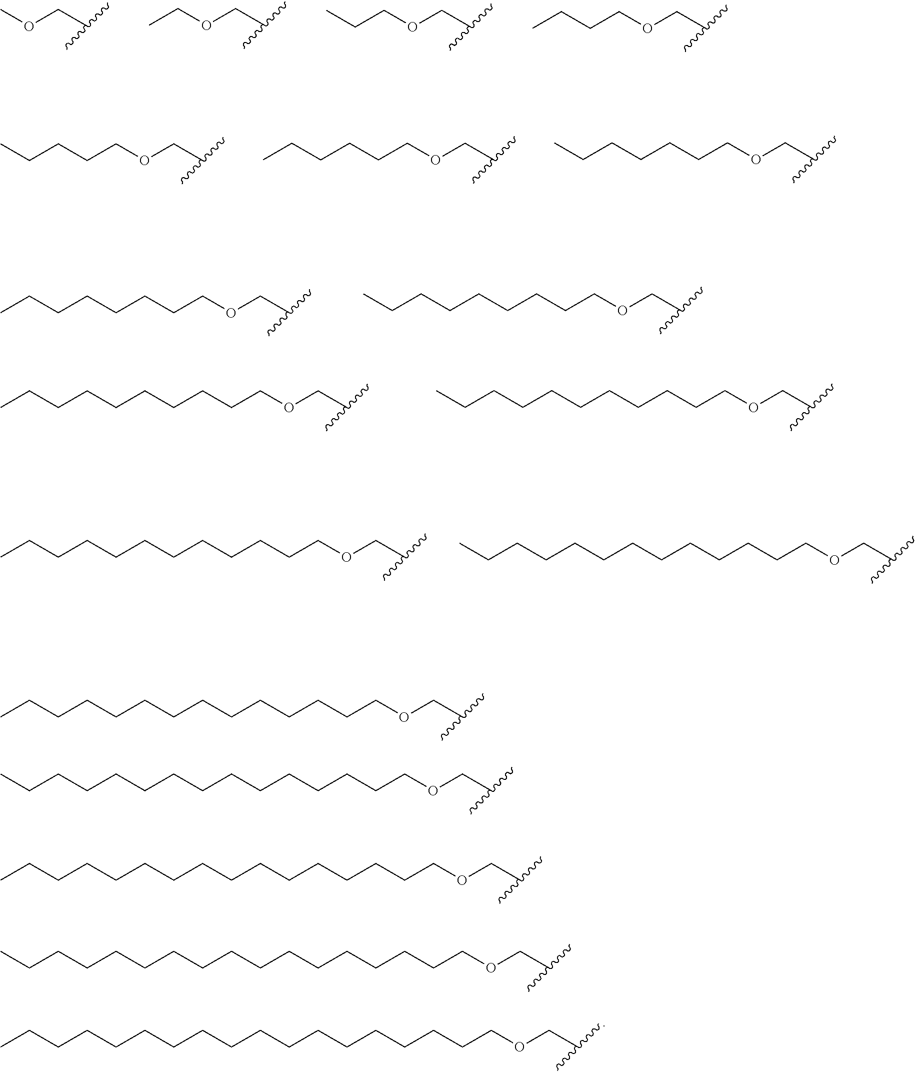

It will be appreciated by one of ordinary skill in the art that the above substituents may have multiple sites of unsaturation, and could be so at any position within the substituent.

In certain embodiments, R₅ is substituted or unsubstituted aryl. In certain embodiments, R₅ is or substituted or unsubstituted heteroaryl.

In certain embodiments, R₅ is fluorinated. In certain embodiments R₅ is a fluorinated aliphatic moiety. In certain embodiments R₅ is perfluorinated. In certain embodiments R₅ is a perfluorinated aliphatic moiety. In certain embodiments, R₅ is a perfluorinated C₁₋₂₀ alkyl group. In certain embodiments, R₅ is selected from the following formulae:

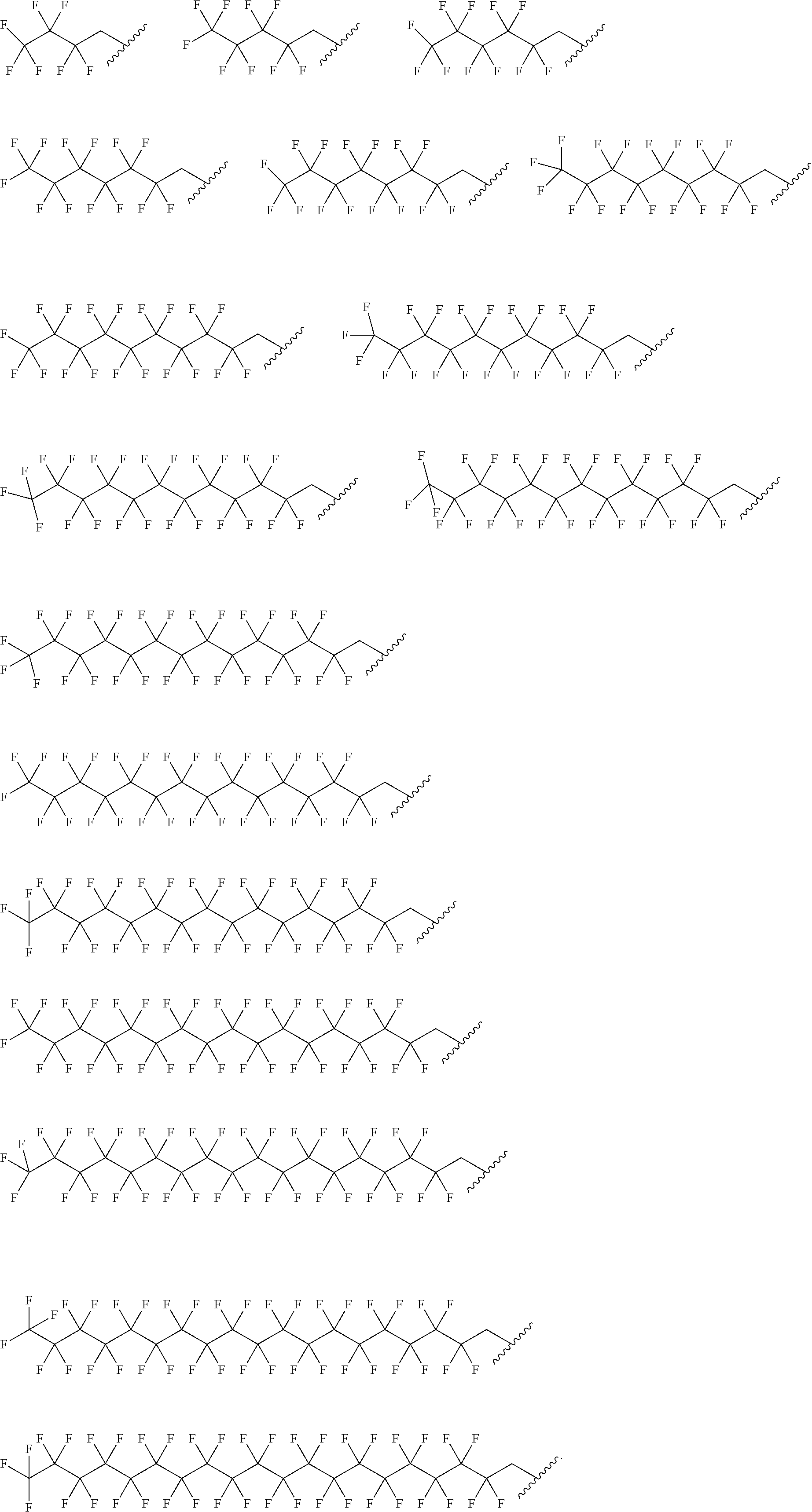

In certain embodiments, R₅ is selected from the following formulae:

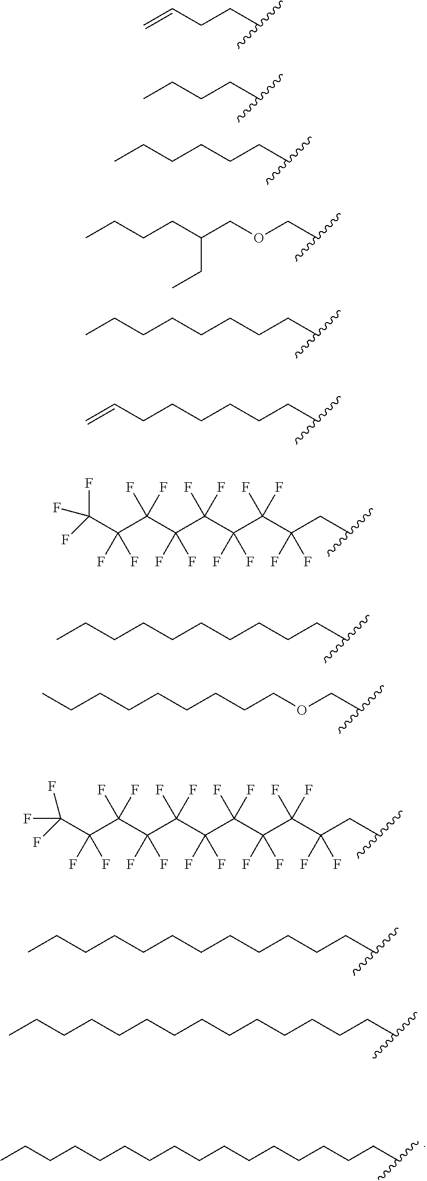

In certain embodiments, each R₅ is independently hydrogen, or C₁-C₆ alkyl. In certain embodiments, each R₅ is hydrogen. In certain embodiments, R₁ and R₃ each R₅ is C₁-C₆ alkyl. In certain embodiments, each R₅ is hydroxyalkyl. In certain embodiments, each R₅ is aminoalkyl. In certain embodiments, two R₅ variables are the same. In certain embodiments, three R₅ variables are the same. In certain embodiments, each R₅ variable is different from the other.

In certain embodiments, x is 1. In certain embodiments, x is 2. In certain embodiments, x is 3. In certain embodiments, x is 4. In certain embodiments, x is 5. In certain embodiments, x is 6. In certain embodiments, x is 7. In certain embodiments, x is 8. In certain embodiments, x is 9. In certain embodiments, x is 10.

In certain embodiments, y is 1. In certain embodiments, y is 2. In certain embodiments, y is 3. In certain embodiments, y is 4. In certain embodiments, y is 5. In certain embodiments, y is 6. In certain embodiments, y is 7. In certain embodiments, y is 8. In certain embodiments, y is 9. In certain embodiments, y is 10.

In certain embodiments, x is 1 and y is 2. In certain embodiments, x is 1 and y is 3. In certain embodiments, x is 1 and y is 4. In certain embodiments, x is 1 and y is 5. In certain embodiments, x is 2 and y is 2. In certain embodiments, x is 2 and y is 3.

In certain embodiments, R_{Y} is hydrogen. In certain embodiments, R_{Y} is substituted or unsubstituted, cyclic or acyclic, branched or unbranched C₁₋₂₀ aliphatic. In certain embodiments, R_{Y} is C₁-C₆ alkyl. In certain embodiments, R_{Y} is substituted or unsubstituted, cyclic or acyclic, branched or unbranched C₁₋₂₀ heteroaliphatic. In certain embodiments, R_{Y} is substituted or unsubstituted aryl. In certain embodiments, R_{Y} is substituted or unsubstituted heteroaryl. In certain embodiments, R_{Y} is

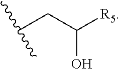

In certain embodiments, R_{Y} is

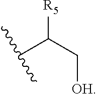

In certain embodiments, R_{Z} is hydrogen. In certain embodiments, R_{Z} is substituted or unsubstituted, cyclic or acyclic, branched or unbranched C₁₋₂₀ aliphatic. In certain embodiments, R_{Y} is C₁-C₆ alkyl. In certain embodiments, R_{Z} is substituted or unsubstituted, cyclic or acyclic, branched or unbranched C₁₋₂₀ heteroaliphatic. In certain embodiments, R_{Z} is substituted or unsubstituted aryl. In certain embodiments, R_{Z} is substituted or unsubstituted heteroaryl. In certain embodiments, R_{Z} is

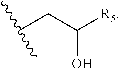

In certain embodiments, R_{Z} is

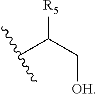

Particular exemplary compounds include:

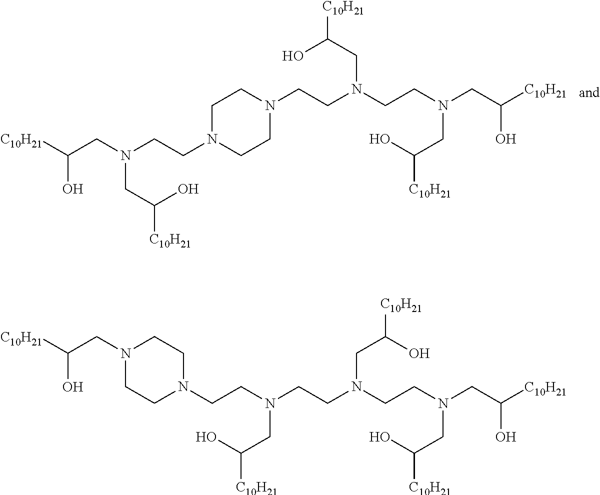

In certain embodiments, the aminoalcohol lipidoid compounds of the present invention comprises a mixture of formulae:

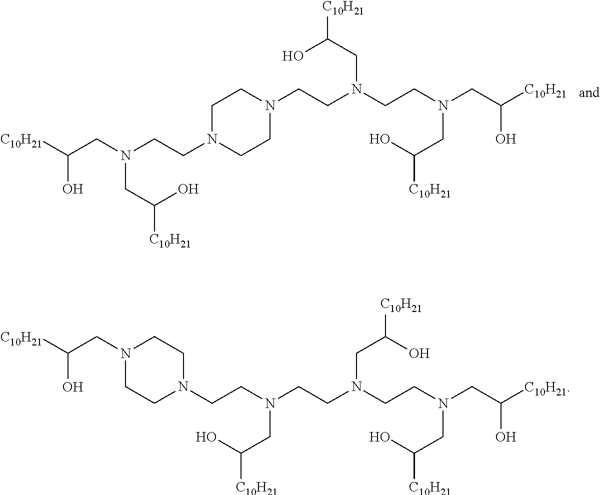

In certain embodiments the aminoalcohol lipidoid compound or composition containing a mixture of aminoalcohol lipidoid compounds is prepared by reacting amine 200

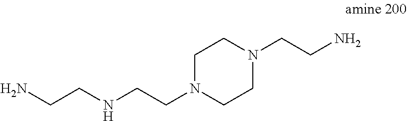 [SMILES: NCCNCCN1CCN(CCN)CC1]

with an epoxide-terminated compound. In certain embodiments, the amine 200-derived aminoalcohol lipidoid compounds (i.e., C12-200) and its various possible isomers are of the formulae below:

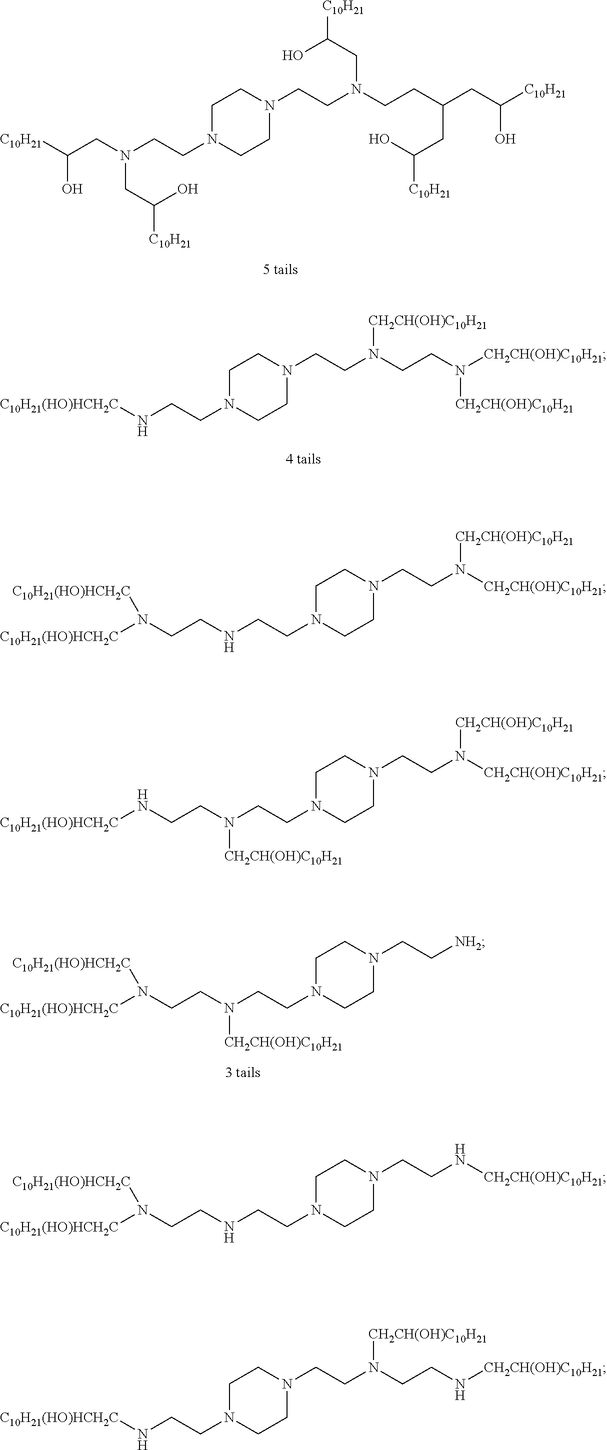

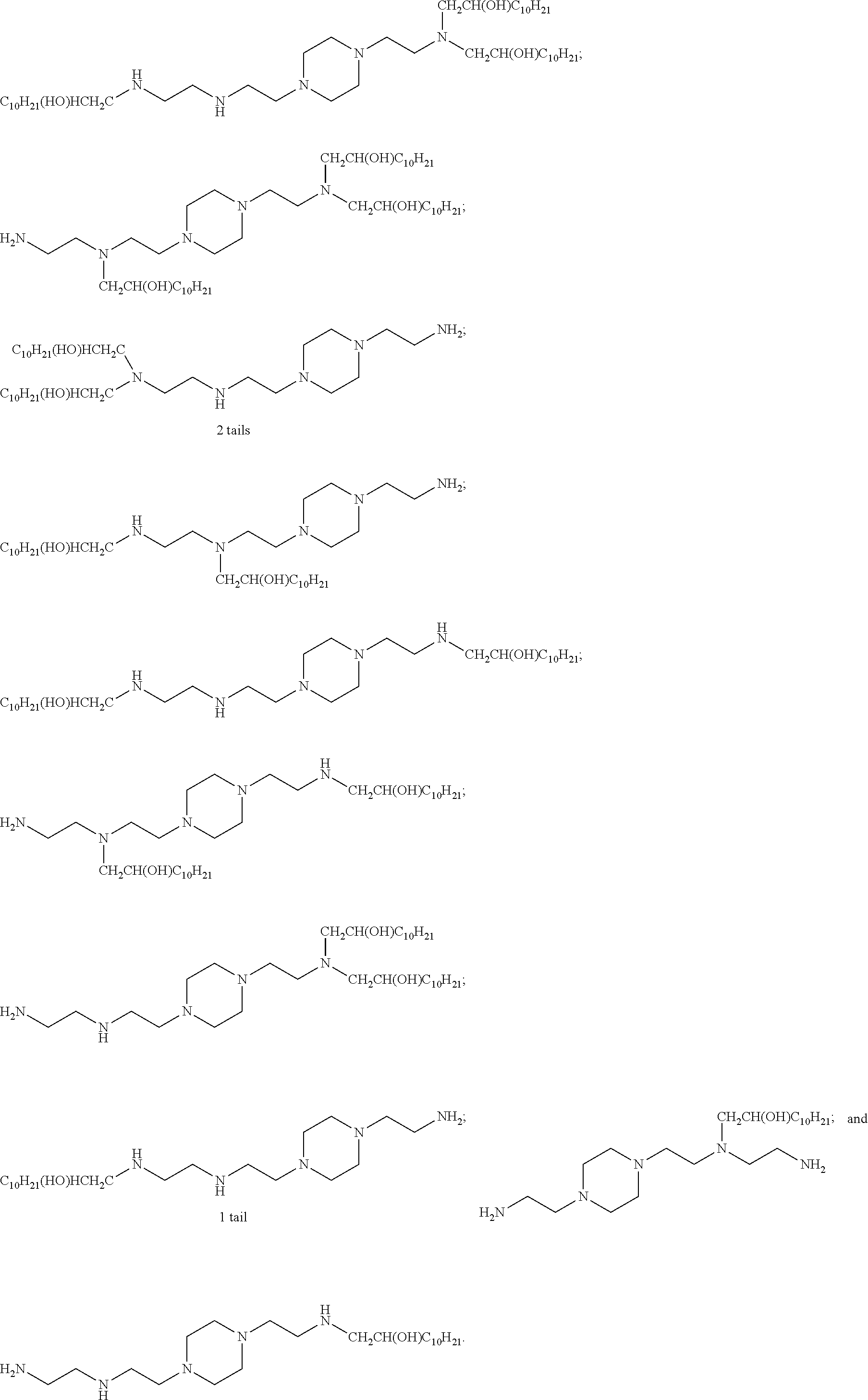

In certain embodiments the aminoalcohol lipidoid composition, is a composition containing one or more of the above aminoalcohol lipidoid compounds.

### Formula (III)

In certain embodiments, the ionizable amino lipid is of Formula (III):

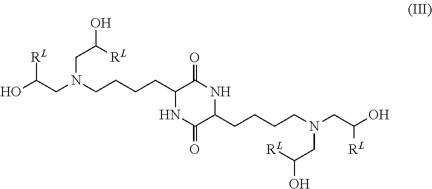

and salts thereof, wherein each instance of R^{L} is independently optionally substituted C₆-C₄₀ alkenyl.

The following chemical group definitions and embodiments apply to compounds of Formula (III) and all subgenera thereof provided herein.

In certain embodiments, the compound of Formula (III) is of the formula:

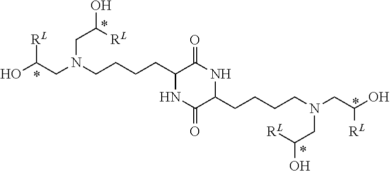

wherein the stereochemistry of each one of the four carbon atoms labeled with “*” is independently S or R.

In certain embodiments, at least two instances of R^{L} is the same group, e.g., for example, in certain embodiments, two instance, three instances, or all four instances, of R^{L}are the same group. In certain embodiments, however, at least one instance of R^{L} is different, e.g., for example, in certain embodiments, at least one, two, three, or all four instances of R^{L}are different groups.

As generally defined herein, each instance of R^{L} is independently optionally substituted C₆-C₄₀ alkenyl. In certain embodiments, at least one (e.g., one, two, three, or each) instance of R^{L} is independently an optionally substituted C₆₋₃₀alkenyl, optionally substituted C₆₋₂₅alkenyl, optionally substituted C₆₋₂₀alkenyl, optionally substituted C₁₀₋₂₅alkenyl, optionally substituted C₁₀₋₂₀alkenyl, optionally substituted C₁₀₋₁₈alkenyl, optionally substituted C₁₀₋₁₆alkenyl, optionally substituted C₁₂₋₃₀alkenyl, optionally substituted C₁₄₋₃₀alkenyl, optionally substituted C₁₆₋₃₀alkenyl, optionally substituted C₁₂₋₁₈alkenyl, optionally substituted C₁₄₋₁₈alkenyl, optionally substituted C₁₆₋₁₈alkenyl, optionally substituted C₁₂₋₁₆alkenyl, or optionally substituted C₁₄₋₁₆alkenyl. In certain embodiments, at least one (e.g., one, two, three, or each) instance of R^{L} is independently an optionally substituted C₁₂ alkenyl, an optionally substituted C₁₃ alkenyl, an optionally substituted C₁₄ alkenyl, an optionally substituted C₁₅alkenyl, an optionally substituted C₁₆ alkenyl, an optionally substituted C₁₇ alkenyl, an optionally substituted C₁₈ alkenyl, an optionally substituted C₁₉ alkenyl, or an optionally substituted C₂₀ alkenyl. In certain embodiments, one or more R^{L} groups, as defined herein, is an unsubstituted alkenyl moiety. In certain embodiments, each of the R^{L} groups, as defined herein, is an unsubstituted alkenyl moiety.

In certain embodiments, one or more R^{L} groups, as defined herein, is an n-alkenyl moiety. For example, in certain embodiments, at least one (e.g., one, two, three, or each) instance of R^{L} is independently optionally substituted C₆-C₄₀ n-alkenyl, e.g., in certain embodiments, at least one (e.g., one, two, three, or each) instance of R^{L} is independently an optionally substituted C₆₋₃₀ n-alkenyl, optionally substituted C₆₋₂₅ n-alkenyl, optionally substituted C₆₋₂₀ n-alkenyl, optionally substituted C₁₀₋₂₅ n-alkenyl, optionally substituted C₁₀₋₂₀ n-alkenyl, optionally substituted C₁₀₋₁₈ n-alkenyl, optionally substituted C₁₀₋₁₆ n-alkenyl, optionally substituted C₁₂₋₃₀ n-alkenyl, optionally substituted C₁₄₋₃₀ n-alkenyl, optionally substituted C₁₆₋₃₀ n-alkenyl, optionally substituted C₁₂₋₁₈ n-alkenyl, optionally substituted C₁₄₋₁₈ n-alkenyl, optionally substituted C₁₆₋₁₈ n-alkenyl, optionally substituted C₁₂₋₁₆ n-alkenyl, or optionally substituted C₁₄₋₁₆ n-alkenyl. In certain embodiments, at least one (e.g., one, two, three, or each) instance of R^{L} is independently an optionally substituted C₁₂ n-alkenyl, an optionally substituted C₁₃ n-alkenyl, an optionally substituted C₁₄ n-alkenyl, an optionally substituted C₁₅n-alkenyl, an optionally substituted C₁₆ n-alkenyl, an optionally substituted C₁₇ n-alkenyl, an optionally substituted C₁₈ n-alkenyl, an optionally substituted C₁₉ n-alkenyl, or an optionally substituted C₂₀ n-alkenyl. In certain embodiments, one or more R^{L} groups, as defined herein, is an unsubstituted n-alkenyl moiety. In certain embodiments, each of the R^{L}groups, as defined herein, is an unsubstituted n-alkenyl moiety.

As understood herein, the alkenyl R^{L} group comprises cis (Z) and/or trans (E) double bonds. It is understood that the designation of cis may also refer to the Z configuration, and the designation of trans may also refer to the E configuration of the double bond if the double bond is tri- or tetra-substituted. In certain embodiments, the only degrees of unsaturation in the group R^{L} are attributed to olefinic (double) bonds. In certain embodiments, at least one (e.g., one, two, three, or each) instance of R^{L} comprises only cis double bonds (and thus no trans double bonds). In certain embodiments, at least one (e.g., one, two, three, or each) instance of R^{L} comprises only trans double bonds (and thus no cis double bonds). In certain embodiments, at least one (e.g., one, two, three, or each) instance of R^{L} comprises 1, 2, or 3 double bonds. In certain embodiments, at least one (e.g., one, two, three, or each) instance of R^{L} comprises 1, 2, or 3 double bonds, and no triple bonds. In certain embodiments, at least one (e.g., one, two, three, or each) instance of R^{L} comprises 2 cis and/or trans double bonds. In certain embodiments, at least one (e.g., one, two, three, or each) instance of R^{L} comprises only cis double bonds. In certain embodiments, trans alkenyl bonds provided in the R^{L} group are specifically excluded. In certain embodiments, each instance of R^{L} comprises only 2 cis double bonds.

In certain embodiments, wherein the at least one (e.g., one, two, three, or each) alkenyl R^{L} group comprises only 1 double bond, the alkenyl R^{L} group is optionally substituted —(C₄₋₁₀alkylene)-(C₂alkenylene)-(C₁₋₂₀alkyl), provided R^{L} comprises no more than 40 linear carbon atoms (in other words, the number of carbon atoms within the linear carbon chain). In certain embodiments, the alkenyl R^{L} group is an —(C₄₋₁₀ n-alkylene)-(C₂alkenylene)-(C₁₋₂₀ n-alkyl), provided R^{L} comprises no more than 40 linear carbon atoms. In certain embodiments, the alkenyl R^{L} group is an optionally substituted —(C₄₋₁₀alkylene)-(cis-C₂alkenylene)-(C₁₋₂₀alkyl) moiety, provided R^{L} comprises no more than 40 linear carbon atoms. In certain embodiments, the alkenyl R^{L} group is an optionally substituted —(C₄₋₁₀ n-alkylene)-(cis-C₂alkenylene)-(C₁₋₂₀ n-alkyl) moiety, provided R^{L} comprises no more than 40 linear carbon atoms. In certain embodiments, R^{L} comprises no more than 30 linear carbon atoms. In certain embodiments, R^{L} comprises between 6 to 40, 10 to 40, 10 to 30, or 10 to 20 linear carbon atoms, inclusive.

For example, in certain embodiments, wherein at least one (e.g., one, two, three, or each) alkenyl R^{L} group comprises only 1 double bond, the at least one (e.g., one, two, three, or each) alkenyl R^{L} group is a group of formula:

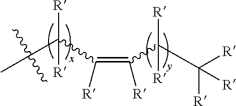

wherein:
x is an integer between 4 and 20, inclusive;
y is an integer between 1 and 20, inclusive; and
each instance of R′ is independently hydrogen, optionally substituted C₁₋₆alkyl, halogen, substituted hydroxyl, substituted thiol, and substituted amino;
provided the group comprises no more than 40 linear carbon atoms.

In certain embodiments, wherein at least one (e.g., one, two, three, or each) alkenyl R^{L} group comprises only 1 double bond, the at least one (e.g., one, two, three, or each) alkenyl R^{L} group is a group of formula:

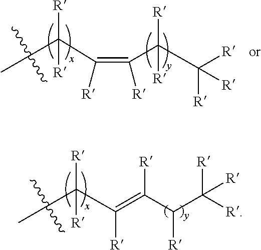

In certain embodiments, each R′ is independently selected from the group consisting of hydrogen, unsubstituted C₁₋₆alkyl (e.g., —CH₃) haloalkyl (e.g., —CF₃), and halogen (e.g., —F). In certain embodiments, each R′ is independently selected from the group consisting of hydrogen and halogen (e.g., —F). In certain embodiments, each R′ is hydrogen.

In certain embodiments, wherein at least one (e.g., one, two, three, or each) alkenyl R^{L} group comprises only 1 double bond, the at least one (e.g., one, two, three, or each) alkenyl R^{L} group is a group of formula:

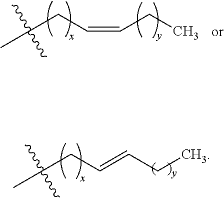

In certain embodiments, x is 4, 5, 6, 7, or 8. In certain embodiments, y is 1, 2, 3, 4, 5, 6, 7, 8, 9, or 10. In certain embodiments, x is 6. In certain embodiments, y is 7.

For example, in certain embodiments, wherein at least one (e.g., one, two, three, or each) alkenyl R^{L} group comprises only 1 double bond, the at least one (e.g., one, two, three, or each) alkenyl R^{L} group is a group of formula:

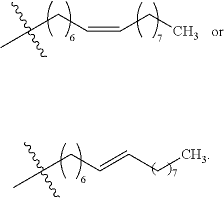

In certain embodiments, wherein the at least one (e.g., one, two, three, or each) alkenyl R^{L} group comprises only 2 double bonds, the alkenyl R^{L} group is optionally substituted —(C₄₋₁₀alkylene)-(C₂alkenylene)-(C₁₋₃alkylene)-(C₂alkenylene)-(C₁₋₂₀alkyl) provided R^{L} comprises no more than 40 linear carbon atoms (in other words, the number of carbon atoms within the linear carbon chain). In certain embodiments, the alkenyl R^{L} group is an —(C₄₋₁₀ n-alkylene)-(C₂alkenylene)-(C₁₋₃ n-alkylene)-(C₂alkenylene)-(C₁₋₂₀ n-alkyl), provided R^{L} comprises no more than 40 linear carbon atoms. In certain embodiments, the alkenyl R^{L} group is an optionally substituted —(C₄₋₁₀alkylene)-(cis-C₂alkenylene)-(C₁₋₃alkylene)-(cis-C₂alkenylene)-(C₁₋₂₀alkyl) moiety, provided R^{L} comprises no more than 40 linear carbon atoms. In certain embodiments, the alkenyl R^{L} group is an optionally substituted —(C₄₋₁₀ n-alkylene)-(cis-C₂alkenylene)-(C₁₋₃ n-alkylene)-(cis-C₂alkenylene)-(C₁₋₂₀ n-alkyl) moiety, provided R^{L} comprises no more than 40 linear carbon atoms. In certain embodiments, R^{L} comprises no more than 30 linear carbon atoms. In certain embodiments, R^{L} comprises between 6 to 40, 10 to 40, 10 to 30, or 10 to 20 linear carbon atoms, inclusive.

For example, in certain embodiments, wherein at least one (e.g., one, two, three, or each) alkenyl R^{L} group comprises only 2 double bonds, the at least one (e.g., one, two, three, or each) alkenyl R^{L} group is a group of formula:

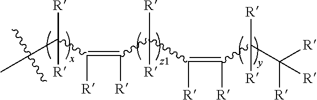

wherein:
x is an integer between 4 and 20, inclusive;
y is an integer between 1 and 20, inclusive;
z1 is 1, 2, or 3;
   and
each instance of R′ is independently hydrogen, optionally substituted C₁₋₆alkyl, halogen, substituted hydroxyl, substituted thiol, and substituted amino;
provided the group comprises no more than 40 linear carbon atoms.

In certain embodiments, wherein at least one (e.g., one, two, three, or each) alkenyl R^{L} group comprises only 2 double bonds, the at least one (e.g., one, two, three, or each) alkenyl R^{L} group is a group of formula:

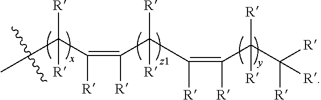

In certain embodiments, each R′ is independently selected from the group consisting of hydrogen, unsubstituted C₁₋₆alkyl (e.g., —CH₃) haloalkyl (e.g., —CF₃), and halogen (e.g., —F). In certain embodiments, each R′ is independently selected from the group consisting of hydrogen and halogen (e.g., —F). In certain embodiments, each R′ is hydrogen.

In certain embodiments, wherein at least one (e.g., one, two, three, or each) alkenyl R^{L} group comprises only 2 double bonds, the at least one (e.g., one, two, three, or each) alkenyl R^{L} group is a group of formula:

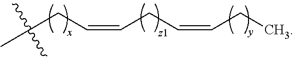

In certain embodiments, x is 4, 5, 6, 7, or 8. In certain embodiments, y is 1, 2, 3, 4, 5, 6, 7, 8, 9, or 10. In certain embodiments, z1 is 1 or 2. In certain embodiments, x is 6. In certain embodiments, y is 4. In certain embodiments, z1 is 1.

For example, in certain embodiments, wherein at least one (e.g., one, two, three, or each) alkenyl R^{L} group comprises only 2 double bond, the at least one (e.g., one, two, three, or each) alkenyl R^{L} group is a group of formula:

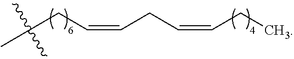

In certain embodiments, wherein the at least one (e.g., one, two, three, or each) alkenyl R^{L} group comprises only 3 double bonds, the alkenyl R^{L} group is an optionally substituted —(C₄₋₁₀alkylene)-(C₂alkenylene)-(C₁₋₃alkylene)-(C₂alkenylene)-(C₁₋₃alkylene)-(C₂alkenylene)-(C₁₋₂₀alkyl) moiety, provided R^{L} comprises no more than 40 linear carbon atoms (in other words, the number of carbon atoms within the linear carbon chain). In certain embodiments, the alkenyl R^{L} group is an —(C₄₋₁₀ n-alkylene)-(C₂alkenylene)-(C₁₋₃ n-alkylene)-(C₂alkenylene)-(C₁₋₃ n-alkylene)-(C₂alkenylene)-(C₁₋₂₀ n-alkyl), provided R^{L}comprises no more than 40 linear carbon atoms. In certain embodiments, the alkenyl R^{L}group is an optionally substituted —(C₄₋₁₀alkylene)-(cis-C₂alkenylene)-(C₁₋₃alkylene)-(cis-C₂alkenylene)-(C₁₋₃alkylene)-(cis-C₂alkenylene)-(C₁₋₂₀alkyl) moiety, provided R^{L} comprises no more than 40 linear carbon atoms. In certain embodiments, the alkenyl R^{L} group is an optionally substituted —(C₄₋₁₀ n-alkylene)-(cis-C₂alkenylene)-(C₁₋₃ n-alkylene)-(cis-C₂alkenylene)-(C₁₋₃ n-alkylene)-(cis-C₂alkenylene)-(C₁₋₂₀ n-alkyl) moiety, provided R^{L}comprises no more than 40 linear carbon atoms. In certain embodiments, R^{L} comprises no more than 30 linear carbon atoms. In certain embodiments, R^{L} comprises between 6 to 40, 10 to 40, 10 to 30, or 10 to 20 linear carbon atoms, inclusive.

For example, in certain embodiments, wherein at least one (e.g., one, two, three, or each) alkenyl R^{L} group comprises only 3 double bonds, the at least one (e.g., one, two, three, or each) alkenyl R^{L} group is a group of formula:

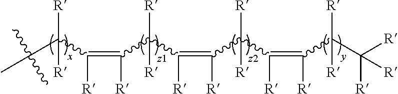

wherein:
x is an integer between 4 and 20, inclusive;
y is an integer between 1 and 20, inclusive;
each instance of z1 and z2 is independently 1, 2, or 3;
   and
each instance of R′ is independently hydrogen, optionally substituted C₁₋₆alkyl, halogen, substituted hydroxyl, substituted thiol, and substituted amino;
provided the group comprises no more than 40 linear carbon atoms.

In certain embodiments, wherein the at least one (e.g., one, two, three, or each) alkenyl R^{L} group comprises only 3 double bonds, the at least one (e.g., one, two, three, or each) alkenyl R^{L} group is a group of formula:

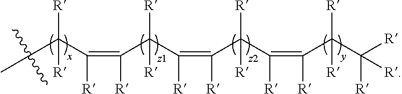

In certain embodiments, each R′ is independently selected from the group consisting of hydrogen, unsubstituted C₁₋₆alkyl (e.g., —CH₃) haloalkyl (e.g., —CF₃), and halogen (e.g., —F). In certain embodiments, each R′ is independently selected from the group consisting of hydrogen and halogen (e.g., —F). In certain embodiments, each R′ is hydrogen.

In certain embodiments, wherein the at least one (e.g., one, two, three, or each) alkenyl R^{L} group comprises only 3 double bonds, the at least one (e.g., one, two, three, or each) alkenyl R^{L} group is a group of formula:

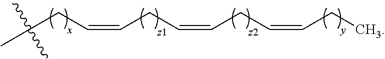

In certain embodiments, x is 4, 5, 6, 7, or 8. In certain embodiments, y is 1, 2, 3, 4, 5, 6, 7, 8, 9, or 10. In certain embodiments, z1 is 1 or 2. In certain embodiments, z2 is 1 or 2. In certain embodiments, x is 6. In certain embodiments, y is 1. In certain embodiments, z1 is 1. In certain embodiments, z2 is 1.

For example, in certain embodiments, wherein the least one (e.g., each) alkenyl R^{L} group comprises only 2 double bonds, the at least one (e.g., one, two, three, or each) alkenyl R^{L} group is a group of formula:

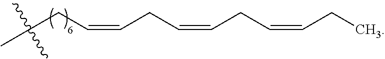

Exemplary compounds of Formula (III) include:
(OF-00), wherein each R^{L} is a group of formula:

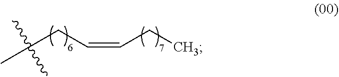

(OF-01), wherein each R^{L} is a group of formula:

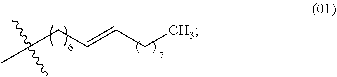

(OF-02), wherein each R^{L} is a group of formula:

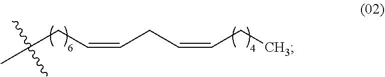

and
(OF-03), wherein each R^{L} is a group of formula:

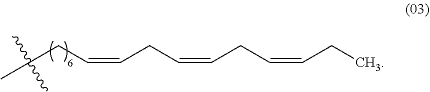

### (b) Sterol

In certain embodiments, the sterol is cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, or a derivative thereof. In some embodiments, the sterol is cholesterol, or a derivative thereof. In certain embodiments, the sterol is cholesterol.

### (c) Phospholipid

In certain embodiments, the phospholid is a phosphoethanolamine or phosphatidylcholine. In some embodiments, the phospholipid is 1,2-distearoyl-sn-glycero-3-phosphorylethanolamine (DSPE), 1,2-distearoyl-sn-glycero-3-phosphocholine (DSPC), or 1,2-dioleoyl-sn-glycero-3-phosphoethanolamine (DOPE). In some embodiments, the phospholipid is a phosphoethanolamine. In certain embodiments, the phospholipid is 1,2-distearoyl-sn-glycero-3-phosphorylethanolamine (DSPE) or phospholipid is 1,2-dioleoyl-sn-glycero-3-phosphoethanolamine (DOPE). In certain embodiments, the phospholipid is 1,2-distearoyl-sn-glycero-3-phosphorylethanolamine (DSPE). In certain embodiments, the phospholipid is 1,2-dioleoyl-sn-glycero-3-phosphoethanolamine (DOPE). In some embodiments, the phospholipid is a phosphatidylcholine. In some embodiments, the phospholipid is 1,2-distearoyl-sn-glycero-3-phosphocholine (DSPC).

### (d) PEG-Lipid

In some embodiments, the PEG-lipid is a PEG-phospholipid or PEG-glyceride lipid. In certain embodiments, the PEG-lipid is 1,2-dimyristoyl-sn-glycero-3-phosphoethanolamine-N-[methoxy(polyethylene glycol)-2000] (C₁₄PEG2000) or 1,2-dimyristoyl-rac-glycero-3-methoxypolyethylene glycol-2000 (DMG-PEG2000).

In certain embodiments, the PEG-lipid is a PEG-phospholipid. In certain embodiments, the PEG-phospholipid is a PEG-phosphoethanolamine. In some embodiments, the PEG-phospholipid is a PEG-phosphatidylcholine.

In certain embodiments, the PEG component has a molecular weight of about 350, about 550, about 750, about 1000, about 2000, about 3000, or about 5000. In some embodiments, the PEG component has a molecular weight of about 750, about 1000, about 2000, about 3000, or about 5000. In certain embodiments, the PEG component has a molecular weight of about 1000, about 2000, or about 3000. In some embodiments, the PEG component has a molecular weight of about 2000.

In certain embodiments, the PEG-lipid is stearoyl-substituted (C₁₈). In some embodiments, the PEG-phospholipid is palmitoyl-substituted (C₁₆). In certain embodiments, the PEG-phospholipid is myristoyl-substituted (C₁₄).

In certain embodiments, the PEG-lipid is selected from the group consisting of 1,2-distearoyl-sn-glycero-3-phosphoethanolamine-N-[methoxy(polyethylene glycol)-5000](C₁₈PEG5000), 1,2-dipalmitoyl-sn-glycero-3-phosphoethanolamine-N-[methoxy(polyethylene glycol)-5000] (C₁₆PEG5000), 1,2-dimyristoyl-sn-glycero-3-phosphoethanolamine-N-[methoxy(polyethylene glycol)-5000] (C₁₄PEG5000), 1,2-distearoyl-sn-glycero-3-phosphoethanolamine-N-[methoxy(polyethylene glycol)-3000](C₁₈PEG3000), 1,2-dipalmitoyl-sn-glycero-3-phosphoethanolamine-N-[methoxy(polyethylene glycol)-3000] (C₁₆PEG3000), 1,2-dimyristoyl-sn-glycero-3-phosphoethanolamine-N-[methoxy(polyethylene glycol)-3000] (C₁₄PEG3000), 1,2-distearoyl-sn-glycero-3-phosphoethanolamine-N-[methoxy(polyethylene glycol)-2000](C₁₈PEG2000), 1,2-dipalmitoyl-sn-glycero-3-phosphoethanolamine-N-[methoxy(polyethylene glycol)-2000] (C₁₆PEG2000), 1,2-dimyristoyl-sn-glycero-3-phosphoethanolamine-N-[methoxy(polyethylene glycol)-2000] (C₁₄PEG2000), 1,2-distearoyl-sn-glycero-3-phosphoethanolamine-N-[methoxy(polyethylene glycol)-1000](C₁₈PEG2000), 1,2-dipalmitoyl-sn-glycero-3-phosphoethanolamine-N-[methoxy(polyethylene glycol)-1000] (C₁₆PEG1000), 1,2-dimyristoyl-sn-glycero-3-phosphoethanolamine-N-[methoxy(polyethylene glycol)-1000] (C₁₄PEG1000), 1,2-distearoyl-sn-glycero-3-phosphoethanolamine-N-[methoxy(polyethylene glycol)-750](C₁₈PEG750), 1,2-dipalmitoyl-sn-glycero-3-phosphoethanolamine-N-[methoxy(polyethylene glycol)-750] (C₁₆PEG750), and 1,2-dimyristoyl-sn-glycero-3-phosphoethanolamine-N-[methoxy(polyethylene glycol)-750] (C₁₄PEG750). In some embodiments, the PEG-lipid is selected from the group consisting of 1,2-distearoyl-sn-glycero-3-phosphoethanolamine-N-[methoxy(polyethylene glycol)-2000] (C₁₈PEG2000), 1,2-dipalmitoyl-sn-glycero-3-phosphoethanolamine-N-[methoxy(polyethylene glycol)-2000] (C₁₆PEG2000), and 1,2-dimyristoyl-sn-glycero-3-phosphoethanolamine-N-[methoxy(polyethylene glycol)-2000](C₁₄PEG2000). In certain embodiments, the PEG-lipid is selected from the group consisting of 1,2-dimyristoyl-sn-glycero-3-phosphoethanolamine-N-[methoxy(polyethylene glycol)-5000] (C₁₄PEG5000), 1,2-dimyristoyl-sn-glycero-3-phosphoethanolamine-N-[methoxy(polyethylene glycol)-3000] (C₁₄PEG3000), 1,2-dimyristoyl-sn-glycero-3-phosphoethanolamine-N-[methoxy(polyethylene glycol)-2000] (C₁₄PEG2000), 1,2-dimyristoyl-sn-glycero-3-phosphoethanolamine-N-[methoxy(polyethylene glycol)-1000](C₁₄PEG1000), and 1,2-dimyristoyl-sn-glycero-3-phosphoethanolamine-N-[methoxy(polyethylene glycol)-750] (C₁₄PEG750). In certain embodiments, the PEG-phospholipid is 1,2-dimyristoyl-sn-glycero-3-phosphoethanolamine-N-[methoxy(polyethylene glycol)-2000] (C₁₄PEG2000).

In some embodiments, the PEG-lipid is a PEG-glyceride lipid. In certain embodiments, the PEG-lipid is 1,2-distearoyl-rac-glycero-3-methoxypolyethylene glycol-2000 (DSG-PEG2000) or 1,2-dimyristoyl-rac-glycero-3-methoxypolyethylene glycol-2000 (DMG-PEG2000). In some embodiments, the PEG-lipid is 1,2-distearoyl-rac-glycero-3-methoxypolyethylene glycol-2000 (DSG-PEG2000). In certain embodiments, the PEG-lipid is 1,2-dimyristoyl-rac-glycero-3-methoxypolyethylene glycol-2000 (DMG-PEG2000).

### Formulations

In some embodiments, the lipids are selected from (a) cKK-E12, (b) cholesterol, (c) DOPE, and (d) DMG-PEG2000. In certain embodiments, the lipids are selected from (a) cKK-E12, (b) cholesterol, (c) DOPE, and (d) C₁₄PEG2000. In certain embodiments, the lipids are selected from (a) cKK-E12, (b) cholesterol, (c) DSPC, and (d) C₁₄PEG2000. In certain embodiments, the lipids are selected from (a) cKK-E12, (b) cholesterol, (c) DSPE, and (d) C₁₄PEG2000. In certain embodiments, the lipids are selected from (a) DOTAP, (b) cholesterol, (c) DOPE, and (d) C₁₄PEG2000.

In some embodiments, the weight ratio of the ionizable amino lipid to the agent is 10:1. In certain embodiments, the weight ratio of the ionizable amino lipid to the agent is 5:1. In some embodiments, the weight ratio of CKK-E12:mRNA is 10:1. In certain embodiments, the weight ratio of CKK-E12:mRNA is 5:1. In some embodiments, the weight ratio of C12-200:mRNA is 10:1. In certain embodiments, the weight ratio of C12-200:mRNA is 5:1. In some embodiments, the weight ratio of OF-02:mRNA is 10:1. In certain embodiments, the weight ratio of OF-02:mRNA is 5:1.

In some embodiments, the molar percentages of lipids are: (a) 35%, (b) 46.5%, (c) 16%, and (d) 2.5%.

### Agents

Agents that are delivered by the systems (e.g., pharmaceutical compositions) described herein may be (e.g., therapeutic or prophylactic), diagnostic, cosmetic, or nutraceutical agents. Any chemical compound to be administered to a subject may be delivered using the complexes, picoparticles, nanoparticles, microparticles, micelles, or liposomes, described herein. In some embodiments, the agent is an organic molecule, inorganic molecule, nucleic acid, protein, peptide, polynucleotide, targeting agent, an isotopically labeled chemical compound, vaccine, an immunological agent, or an agent useful in bioprocessing (e.g., intracellular manufacturing of proteins, such as a cell's bioprocessing of a commercially useful chemical or fuel). For example, intracellular delivery of an agent may be useful in bioprocessing by maintaining the cell's health and/or growth, e.g., in the manufacturing of proteins. Any chemical compound to be administered to a subject or contacted with a cell may be delivered to the subject or cell using the compositions.

Exemplary agents that may be included in a composition described herein include, but are not limited to, small molecules, organometallic compounds, polynucleotides, proteins, peptides, carbohydrates, monosaccharides, oligosaccharides, polysaccharides, nucleoproteins, mucoproteins, lipoproteins, small molecules linked to proteins, glycoproteins, steroids, nucleotides, oligonucleotides, polynucleotides, nucleosides, antisense oligonucleotides, lipids, hormones, vitamins, cells, metals, targeting agents, isotopically labeled chemical compounds, drugs (e.g., compounds approved for human or veterinary use by the U.S. Food and Drug Administration as provided in the Code of Federal Regulations), vaccines, immunological agents, agents useful in bioprocessing, and mixtures thereof. The targeting agents are described in more detail herein. In certain embodiments, the agents are nutraceutical agents. In certain embodiments, the agents are pharmaceutical agents (e.g., a therapeutic or prophylactic agent). In certain embodiments, the agent is an antibiotic agent (e.g., an anti-bacterial, anti-viral, or anti-fungal agent), anesthetic, steroidal agent, anti-proliferative agent, anti-inflammatory agent, anti-angiogenesis agent, anti-neoplastic agent, anti-cancer agent, anti-diabetic agent, antigen, vaccine, antibody, decongestant, antihypertensive, sedative, birth control agent, progestational agent, anti-cholinergic, analgesic, immunosuppressant, anti-depressant, anti-psychotic, β-adrenergic blocking agent, diuretic, cardiovascular active agent, vasoactive agent, non-steroidal, nutritional agent, anti-allergic agent, or pain-relieving agent. Vaccines may comprise isolated proteins or peptides, inactivated organisms and viruses, dead organisms and viruses, genetically altered organisms or viruses, and cell extracts. Therapeutic and prophylactic agents may be combined with interleukins, interferon, cytokines, and adjuvants such as cholera toxin, alum, and Freund's adjuvant, etc.

In certain embodiments, an agent to be delivered or used in a composition described herein is a polynucleotide. In certain embodiments, the agent is plasmid DNA (pDNA). In certain embodiments, the agent is single-stranded DNA (ssDNA), double-stranded DNA (dsDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA. In certain embodiments, the agent is RNA. In certain embodiments, the agent is small interfering RNA (siRNA). In certain embodiments, the agent is messenger RNA (mRNA). In certain embodiments, the agent is single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, the agent is an RNA that carries out RNA interference (RNAi). The phenomenon of RNAi is discussed in greater detail, for example, in the following references: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553. In certain embodiments, upon delivery of an RNA into a subject, tissue, or cell, the RNA is able to interfere with the expression of a specific gene in the subject, tissue, or cell. In certain embodiments, the agent is a pDNA, siRNA, mRNA, or a combination thereof.

In certain embodiments, the polynucleotide may be provided as an antisense agent or RNAi. See, e.g., Fire et al., Nature 391:806-811, 1998. Antisense therapy is meant to include, e.g., administration or in situ provision of single- or double-stranded polynucleotides, or derivatives thereof, which specifically hybridize, e.g., bind, under cellular conditions, with cellular mRNA and/or genomic DNA, or mutants thereof, so as to inhibit the expression of the encoded protein, e.g., by inhibiting transcription and/or translation. See, e.g., Crooke, “Molecular mechanisms of action of antisense drugs,” Biochim. Biophys. Acta 1489(1):31-44, 1999; Crooke, “Evaluating the mechanism of action of anti-proliferative antisense drugs,” Antisense Nucleic Acid Drug Dev. 10(2):123-126, discussion 127, 2000; Methods in Enzymology volumes 313-314, 1999. The binding may be by conventional base pair complementarity, or, for example, in the case of binding to DNA duplexes, through specific interactions in the major groove of the double helix (i.e., triple helix formation). See, e.g., Chan et al., J. Mol. Med. 75(4):267-282, 1997.

In some embodiments, pDNA, siRNA, dsRNA, shRNA, miRNA, mRNA, tRNA, asRNA, and/or RNAi can be designed and/or predicted using one or more of a large number of available algorithms. To give but a few examples, the following resources can be utilized to design and/or predict polynucleotides: algorithms found at Alnylum Online; Dharmacon Online; OligoEngine Online; Molecula Online; Ambion Online; BioPredsi Online; RNAi Web Online; Chang Bioscience Online; Invitrogen Online; LentiWeb Online GenScript Online; Protocol Online; Reynolds et al., 2004, Nat. Biotechnol., 22:326; Naito et al., 2006, Nucleic Acids Res., 34:W448; Li et al., 2007, RNA, 13:1765; Yiu et al., 2005, Bioinformatics, 21:144; and Jia et al., 2006, BMC Bioinformatics, 7: 271.

The polynucleotide included in a composition may be of any size or sequence, and they may be single- or double-stranded. In certain embodiments, the polynucleotide includes at least about 30, at least about 100, at least about 300, at least about 1,000, at least about 3,000, or at least about 10,000 base pairs. In certain embodiments, the polynucleotide includes less than about 10,000, less than about 3,000, less than about 1,000, less than about 300, less than about 100, or less than about 30 base pairs. Combinations of the above ranges (e.g., at least about 100 and less than about 1,000) are also within the scope of the invention. The polynucleotide may be provided by any means known in the art. In certain embodiments, the polynucleotide is engineered using recombinant techniques. See, e.g., Ausubel et al., Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989). The polynucleotide may also be obtained from natural sources and purified from contaminating components found normally in nature. The polynucleotide may also be chemically synthesized in a laboratory. In certain embodiments, the polynucleotide is synthesized using standard solid phase chemistry. The polynucleotide may be isolated and/or purified. In certain embodiments, the polynucleotide is substantially free of impurities. In certain embodiments, the polynucleotide is at least about 50%, at least about 60%, at least about 70%, at least about 80%, at least about 90%, at least about 95%, or at least about 99% free of impurities.

The polynucleotide may be modified by physical, chemical, and/or biological means. The modifications include methylation, phosphorylation, and end-capping, etc. In certain embodiments, the modifications lead to increased stability of the polynucleotide.

Wherever a polynucleotide is employed in the composition, a derivative of the polynucleotide may also be used. These derivatives include products resulted from modifications of the polynucleotide in the base moieties, sugar moieties, and/or phosphate moieties of the polynucleotide. Modified base moieties include, but are not limited to, 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine. Modified sugar moieties include, but are not limited to, 2′-fluororibose, ribose, 2′-deoxyribose, 3′-azido-2′,3′-dideoxyribose, 2′,3′-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5′-N-phosphoramidite linkages. Combinations of the various modifications may be used in a single polynucleotide. These modified polynucleotides may be provided by any means known in the art; however, as will be appreciated by those of skill in the art, the modified polynucleotides may be prepared using synthetic chemistry in vitro.

The polynucleotide described herein may be in any form, such as a circular plasmid, a linearized plasmid, a cosmid, a viral genome, a modified viral genome, and an artificial chromosome.

The polynucleotide described herein may be of any sequence. In certain embodiments, the polynucleotide encodes a protein or peptide. The encoded protein may be an enzyme, structural protein, receptor, soluble receptor, ion channel, active (e.g., pharmaceutically active) protein, cytokine, interleukin, antibody, antibody fragment, antigen, coagulation factor, albumin, growth factor, hormone, and insulin, etc. The polynucleotide may also comprise regulatory regions to control the expression of a gene. These regulatory regions may include, but are not limited to, promoters, enhancer elements, repressor elements, TATA boxes, ribosomal binding sites, and stop sites for transcription, etc. In certain embodiments, the polynucleotide is not intended to encode a protein. For example, the polynucleotide may be used to fix an error in the genome of the cell being transfected.

In certain embodiments, the polynucleotide described herein comprises a sequence encoding an antigenic peptide or protein. A composition containing the polynucleotide can be delivered to a subject to induce an immunologic response sufficient to decrease the chance of a subsequent infection and/or lessen the symptoms associated with such an infection. The polynucleotide of these vaccines may be combined with interleukins, interferon, cytokines, and/or adjuvants described herein.

The antigenic protein or peptides encoded by the polynucleotide may be derived from bacterial organisms, such as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, and Camphylobacter jejuni; from viruses, such as smallpox virus, influenza A virus, influenza B virus, respiratory syncytial virus, parainfluenza virus, measles virus, HIV virus, varicella-zoster virus, herpes simplex 1 virus, herpes simplex 2 virus, cytomegalovirus, Epstein-Barr virus, rotavirus, rhinovirus, adenovirus, papillomavirus, poliovirus, mumps virus, rabies virus, rubella virus, coxsackieviruses, equine encephalitis virus, Japanese encephalitis virus, yellow fever virus, Rift Valley fever virus, hepatitis A virus, hepatitis B virus, hepatitis C virus, hepatitis D virus, and hepatitis E virus; and from fungal, protozoan, or parasitic organisms, such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, and Schistosoma mansoni.

In certain embodiments, the agent is erythropoietin (EPO), e.g., recombinant human erythropoietin (rhEPO). Erythropoietin is an essential hormone for red blood cell production, and may be used in treating hematological diseases, e.g., anemia., such as anemia resulting from chronic kidney disease, chemotherapy induced anemia in patients with cancer, inflammatory bowel disease (Crohn's disease and ulcerative colitis) and myelodysplasia from the treatment of cancer (chemotherapy and radiation). Recombinant human erythropoietins available for use include EPOGEN/PROCRIT (Epoetin alfa, rINN) and ARANESP (Darbepoetin alfa, rINN).

An agent described herein may be non-covalently (e.g., complexed or encapsulated) attached to a compound as described herein, or included in a composition described herein. In certain embodiments, upon delivery of the agent into a cell, the agent is able to interfere with the expression of a specific gene in the cell.

In certain embodiments, the agent in a composition that is delivered to a subject in need thereof may be a mixture of two or more agents that may be useful as, e.g., combination therapies. The compositions including the two or more agents can be administered to achieve a synergistic effect. In certain embodiments, the compositions including the two or more agents can be administered to improve the activity and/or bioavailability, reduce and/or modify the metabolism, inhibit the excretion, and/or modify the distribution within the body of a subject, of each one of the two or more agents. It will also be appreciated that the therapy employed may achieve a desired effect for the same disorder, and/or it may achieve different effects.

The compositions (e.g., pharmaceutical compositions) can be administered concurrently with, prior to, or subsequent to the one or more agents (e.g., pharmaceutical agents). The two or more agents may be useful for treating and/or preventing a same disease or different diseases described herein. Each one of the agents may be administered at a dose and/or on a time schedule determined for that agent. The agents may also be administered together with each other and/or with the composition described herein in a single dose or administered separately in different doses. The particular combination to employ in a regimen will take into account compatibility of the agents and/or the desired therapeutic and/or prophylactic effect to be achieved. In general, it is expected that the agents utilized in combination be utilized at levels that do not exceed the levels at which they are utilized individually. In some embodiments, the levels utilized in combination will be lower than those utilized individually.

### Targeting Agents

Since it is often desirable to target a particular cell, collection of cells, or tissue, compounds provided herein, and the complexes, liposomes, micelles, and particles (e.g., microparticles and nanoparticles) thereof, may be modified to include targeting moieties. For example, a compound provided herein may include a targeting moiety. A variety of agents or regions that target particular cells are known in the art. See, e.g., Cotten et al., Methods Enzym. 217:618, 1993. The targeting agent may be included throughout a particle of a compound provided herein or may be only on the surface of the particle. The targeting agent may be a protein, peptide, carbohydrate, glycoprotein, lipid, small molecule, or polynucleotide, etc. The targeting agent may be used to target specific cells or tissues or may be used to promote endocytosis or phagocytosis of the particle. Examples of targeting agents include, but are not limited to, antibodies, fragments of antibodies, proteins, peptides, carbohydrates, receptor ligands, sialic acid, and aptamers, etc. If the targeting agent is included throughout a particle, the targeting agent may be included in the mixture that is used to form the particle. If the targeting agent is only on the surface of a particle, the targeting agent may be associated with (e.g., by covalent or non-covalent (e.g., electrostatic, hydrophobic, hydrogen bonding, van der Waals, π-π stacking) interactions) the formed particle using standard chemical techniques.

### Particles

A composition including a compound provided herein and an agent may be in the form of a particle. In certain embodiments, the compound provided herein and agent form a complex, and the complex is in the form of a particle. In certain embodiments, the compound provided herein encapsulates the agent and is in the form of a particle. In certain embodiments, the compound provided herein is mixed with the agent, and the mixture is in the form of a particle.

In certain embodiments, a complex of a compound provided herein and an agent in a composition of is in the form of a particle. In certain embodiments, the particle is a microparticle (i.e., particle having a characteristic dimension of less than about 1 millimeter and at least about 1 micrometer, where the characteristic dimension of the particle is the smallest cross-sectional dimension of the particle). In certain embodiments, the particle is a nanoparticle (i.e., a particle having a characteristic dimension of less than about 1 micrometer and at least about 1 nanometer, where the characteristic dimension of the particle is the smallest cross-sectional dimension of the particle). In certain embodiments, the average diameter of the particle is at least about 10 nm, at least about 30 nm, at least about 100 nm, at least about 300 nm, at least about 1 μm, at least about 3 μm, at least about 10 μm, at least about 30 μm, at least about 100 μm, at least about 300 μm, or at least about 1 mm. In certain embodiments, the average diameter of the particle is less than about 1 mm, less than about 300 μm, less than about 100 μm, less than about 30 μm less than about 10 μm, less than about 3 μm, less than about 1 μm, less than about 300 nm, less than about 100 nm, less than about 30 nm, or less than about 10 nm. Combinations of the above ranges (e.g., at least about 100 nm and less than about 1 μm) are also within the scope of the present invention.

The particles described herein may include additional materials such as polymers (e.g., synthetic polymers (e.g., PEG, PLGA) and natural polymers (e.g., phospholipids)). In certain embodiments, the additional materials are approved by a regulatory agency, such as the U.S. FDA, for human and veterinary use.

The particles may be prepared using any method known in the art, such as precipitation, milling, spray drying, single and double emulsion solvent evaporation, solvent extraction, phase separation, and simple and complex coacervation. In certain embodiments, methods of preparing the particles are the double emulsion process and spray drying. The conditions used in preparing the particles may be altered to yield particles of a desired size or property (e.g., hydrophobicity, hydrophilicity, external morphology, “stickiness”, shape, polydispersity, etc.). The method of preparing the particle and the conditions (e.g., solvent, temperature, concentration, and air flow rate, etc.) used may also depend on the agent being complexed, encapsulated, or mixed, and/or the composition of the matrix.

Methods developed for making particles for delivery of agents that are included in the particles are described in the literature. See, e.g., Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al., Reactive Polymers 6:275-283, 1987; Mathiowitz et al., J. Appl. Polymer Sci. 35:755-774, 1988.

If the particles prepared by any of the above methods have a size range outside of the desired range, the particles can be sized, for example, using a sieve. The particles may also be coated. In certain embodiments, the particles are coated with a targeting agent. In certain embodiments, the particles are coated with a surface-altering agent. In some embodiments, the particles are coated to achieve desirable surface properties (e.g., a particular charge).

In certain embodiments, the polydispersity index (PDI, determined by dynamic light scattering) of the particles described herein (e.g., particles included in a composition described herein) is between 0.01 and 0.9, between 0.1 and 0.9, between 0.1 and 0.7, between 0.1 and 0.5, between 0.01 and 0.4, between 0.03 and 0.4, between 0.1 and 0.4, between 0.01 and 0.3, between 0.03 and 0.3, or between 0.1 and 0.3.

### Micelles and Liposomes

A composition including one or more compounds provided herein and an agent may be in the form of a micelle or liposome. In certain embodiments, the compound provided herein is in the form of a micelle or liposome. In certain embodiments, the agent is in the form of a micelle or liposome. In certain embodiments, the compound provided herein and agent form a complex, and the complex is in the form of a micelle or liposome. In certain embodiments, the compound provided herein encapsulates the agent and is in the form of a micelle or liposome. In certain embodiments, the compound provided herein is mixed with the agent, and the mixture is in the form of a micelle or liposome. Micelles and liposomes are particularly useful in delivering an agent, such as a hydrophobic agent. When the micelle or liposome is complexed with (e.g., encapsulates or covers) a polynucleotide, the resulting complex may be referred to as a “lipoplex.” Many techniques for preparing micelles and liposomes are known in the art, and any such method may be used herein to make micelles and liposomes.

In certain embodiments, liposomes are formed through spontaneous assembly. In some embodiments, liposomes are formed when thin lipid films or lipid cakes are hydrated and stacks of lipid crystalline bilayers become fluid and swell. The hydrated lipid sheets detach during agitation and self-close to form large, multilamellar vesicles (LMV). This prevents interaction of water with the hydrocarbon core of the bilayers at the edges. Once these liposomes have formed, reducing the size of the liposomes can be modified through input of sonic energy (sonication) or mechanical energy (extrusion). See, e.g., Walde, P. “Preparation of Vesicles (Liposomes)” In Encylopedia of Nanoscience and Nanotechnology; Nalwa, H. S. Ed. American Scientific Publishers: Los Angeles, 2004; Vol. 9, pp. 43-79; Szoka et al., “Comparative Properties and Methods of Preparation of Lipid Vesicles (Liposomes)” Ann. Rev. Biophys. Bioeng. 9:467-508, 1980; each of which is incorporated herein by reference. The preparation of lipsomes may involve preparing a compound provided herein for hydration, hydrating the compound with agitation, and sizing the vesicles to achieve a homogenous distribution of liposomes. A compound provided herein may be first dissolved in an organic solvent in a container to result in a homogeneous mixture. The organic solvent is then removed to form a polymer-derived film. This polymer-derived film is thoroughly dried to remove residual organic solvent by placing the container on a vacuum pump for a period of time. Hydration of the polymer-derived film is accomplished by adding an aqueous medium and agitating the mixture. Disruption of LMV suspensions using sonic energy typically produces small unilamellar vesicles (SUV) with diameters in the range of 15-50 nm. Lipid extrusion is a technique in which a lipid/polymer suspension is forced through a polycarbonate filter with a defined pore size to yield particles having a diameter near the pore size of the filter used. Extrusion through filters with 100 nm pores typically yields large, unilamellar polymer-derived vesicles (LUV) with a mean diameter of 120-140 nm. In certain embodiments, the amount of a compound provided herein in the liposome ranges from about 30 mol % to about 80 mol %, from about 40 mol % to about 70 mol %, or from about 60 mol % to about 70 mol %. In certain embodiments, the compound provided herein employed further complexes an agent, such as a polynucleotide. In such embodiments, the application of the liposome is the delivery of the polynucleotide.

The following scientific papers described other methods for preparing liposomes and micelles: Narang et al., “Cationic Lipids with Increased DNA Binding Affinity for Nonviral Gene Transfer in Dividing and Nondividing Cells,” Bioconjugate Chem. 16:156-68, 2005; Hofland et al., “Formation of stable cationic lipid/DNA complexes for gene transfer,” Proc. Natl. Acad. Sci. USA 93:7305-7309, July 1996; Byk et al., “Synthesis, Activity, and Structure—Activity Relationship Studies of Novel Cationic Lipids for DNA Transfer,” J. Med Chem. 41(2):224-235, 1998; Wu et al., “Cationic Lipid Polymerization as a Novel Approach for Constructing New DNA Delivery Agents,” Bioconjugate Chem. 12:251-57, 2001; Lukyanov et al., “Micelles from lipid derivatives of water-soluble polymers as delivery systems for poorly soluble drugs,” Advanced Drug Delivery Reviews 56:1273-1289, 2004; Tranchant et al., “Physicochemical optimisation of plasmid delivery by cationic lipids,” J. Gene Med. 6:S24-S35, 2004; van Balen et al., “Liposome/Water Lipophilicity: Methods, Information Content, and Pharmaceutical Applications,” Medicinal Research Rev. 24(3):299-324, 2004.

### Kits

Also contemplated herein are kits (e.g., packs). The kits provided may comprise a composition as described herein and a container (e.g., a vial, ampule, bottle, syringe, and/or dispenser package, or other suitable container). In some embodiments, provided kits may optionally further include a second container comprising an excipient for dilution or suspension of the composition. In some embodiments, the composition provided in the first container and the composition provided in the second container are combined to form one unit dosage form. In certain embodiments, the kits further include instructions for administering the composition. The kits may also include information as required by a regulatory agency such as the U.S. Food and Drug Administration (FDA). In certain embodiments, the information included in the kits is prescribing information. In certain embodiments, the kits, including the instructions, provide for treating and/or preventing a disease, disorder, or condition described herein. The kit may include one or more agents described herein as a separate composition.

### EXAMPLES

In order that the invention described herein may be more fully understood, the following examples are set forth. It should be understood that these examples are for illustrative purposes only and are not to be construed as limiting this invention in any manner.

### Example 1

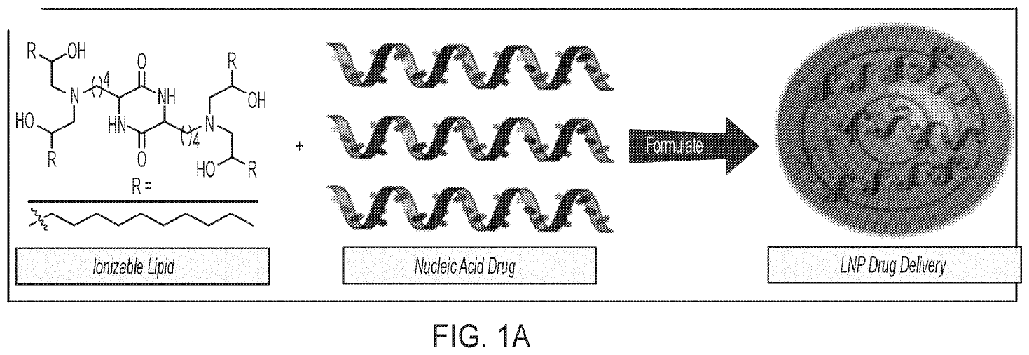

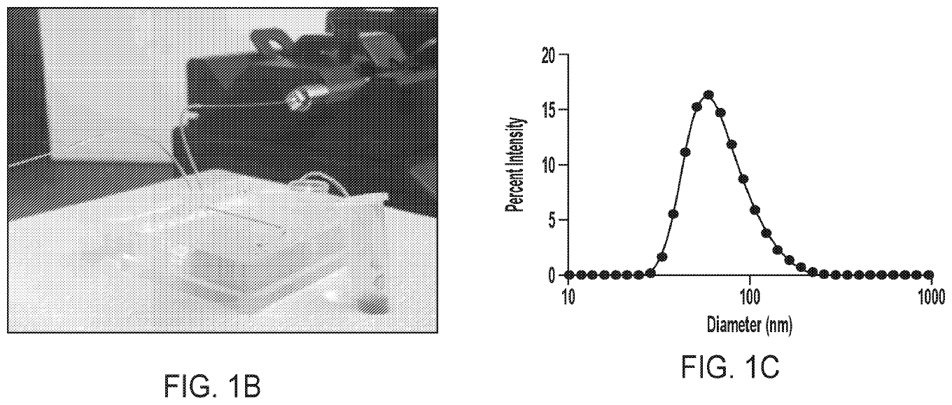

This system proposes the use of lipid nanoparticles (LNP) encapsulating therapeutic cargo as a novel drug delivery vehicle for selective uptake in microglia. Towards this end, LNP formulations capable of transfecting microglia with mRNA encoding for reporter genes in vitro and in vivo were identified. These LNP formulations utilize ApoE-dependent mechanisms of uptake for intracellular delivery of the encapsulated cargo.² Although mRNA has acted as the initial cargo, other types of therapeutic nucleic acids (siRNA, ASO, DNA) or small molecule drugs can be encapsulated and delivered to microglia utilizing the same LNP technology platform (FIG. 1A)³⁻⁵. The formulation will be optimized by varying the ratio of individual molecular components in order improve cell type specificity and delivery. Additional work can be performed through chemical modification of the LNP surface with microglia-specific targeting moieties. Successful development of this technology would greatly expand upon traditional treatment options and develop new paradigms for addressing neurodegeneration disorders and neuroinflammation.

LNPs were formulated using four standard lipid nanoparticle components: cholesterol, DOPE phospholipid, DMG-PEG2000 pegylated lipid, and ionizable lipid cKK-E12. Cholesterol (Sigma Aldrich), DOPE (Avanti Polar Lipids), and DMG-PEG2000 (Avanti Polar Lipids) are commercially available. cKK-E12 was synthesized according to previously published work². These lipids were combined in a 46.5:16:2.5:35 molar ratio in ethanol and mixed in a microfluidic device with firefly luciferase mRNA (Trilink Biotech) in 10 mM citrate buffer pH 3.0 in a defined 1:3 volume ratio (FIG. 1B)⁶. Additional chemical components with varying structural modifications, including alternative phospholipids or modified cholesterols, can be incorporated utilizing the same microfluidic mixing process⁷. LNPs were dialyzed in PBS and characterized for physical parameters including polydispersity, size, and encapsulation rate (FIG. 1C).

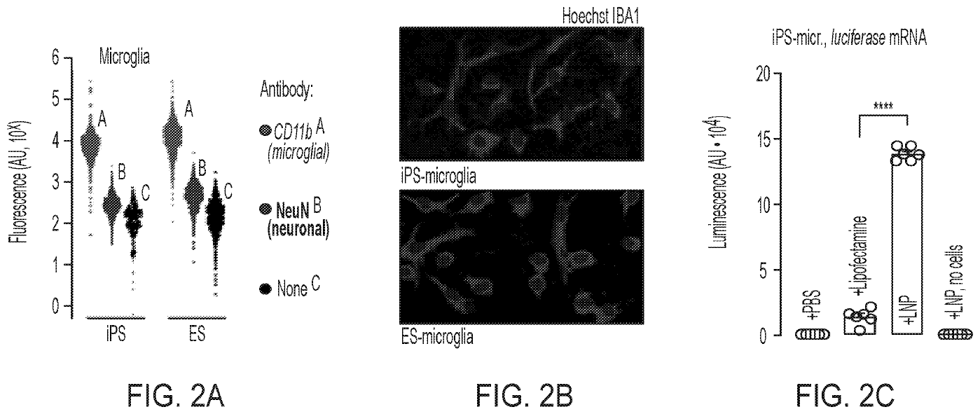

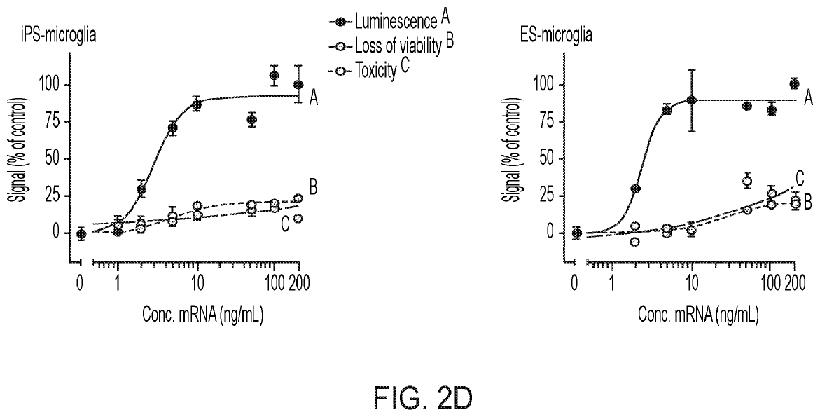

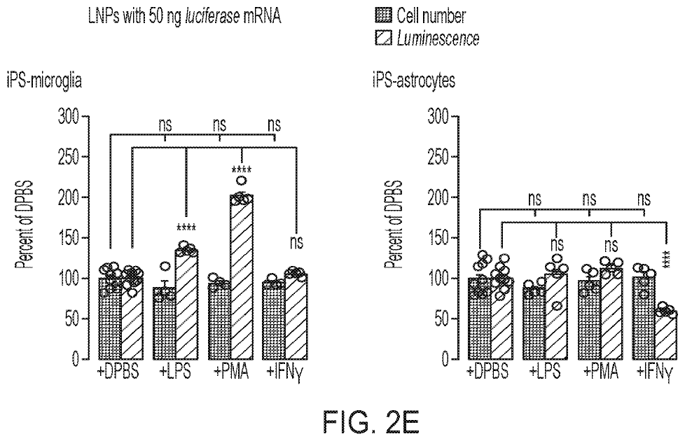

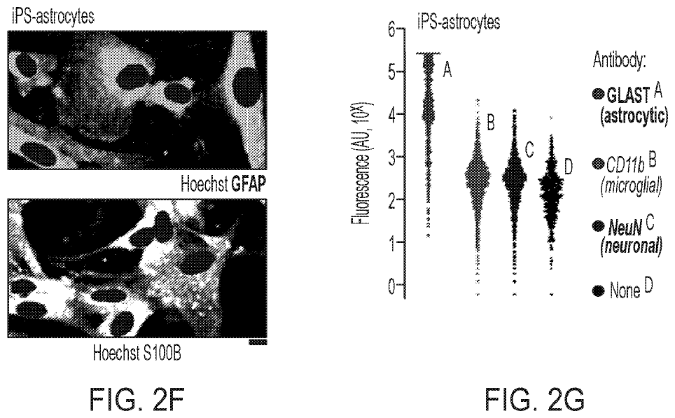

Prior developed methods were utilized to generate induced pluripotent stem cell cultures (iPSC) containing differentiated microglia or astrocytes (FIG. 2A,2B,2F,2G). Cells were initially screened at 50 ng mRNA per 10 000 cells in a 96 well plate. Subsequent dose-dependent studies varied the encapsulated mRNA between 1 ng and 200 ng mRNA per 10 000 cells. Upon treatment with LNP-encapsulated mRNA, the iPS- and ES-microglia expressed significant levels of bioluminescence (FIG. 2C). Dose-dependent studies revealed that maximal expression was achieved at a 10 ng mRNA per 10 000 cell concentration, outperforming commercially available transfection agent Lipofectamine (FIG. 2D). Inflammatory conditions were simulated by adding agents such as lipopolysaccharide (LPS) or phorbol myristate acetate (PMA) to the media of iPS-microglia. With the exception of IFNg treatment, LNP transfection was improved in inflammatory microglia as compared to untreated microglia. These results suggest that LNPs could be used to target disease-associated inflammatory microglia. On the other hand, iPS-astrocytes treated with identical pro-inflammatory signals did not achieve similar increases in LNP transfection (FIG. 2E).

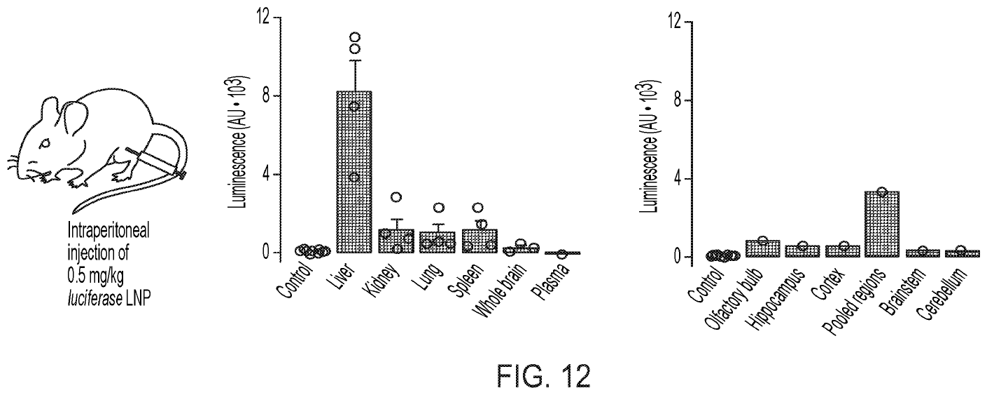

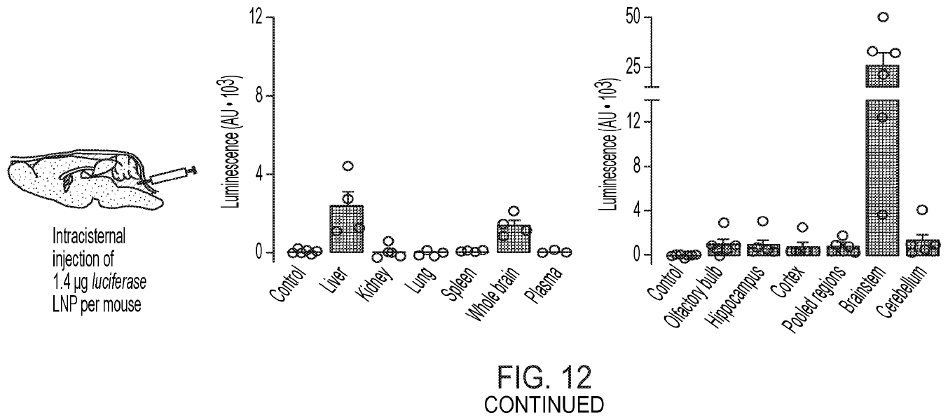

For pilot in vivo experiments, LNP were concentrated using a centrifugal filter prior to administration in mice. Wildtype (C57BL6/J) or P301STau mice were dosed at 0.5 mg/kg intraperitoneally or with 1.4 pg through an intracisternal injection into the cerebrospinal fluid via the foramen magnum. After 24 hours, mice anesthetized and peripheral organs or the brain was resected for tissue analysis (FIG. 12). Brains were dissected to identify transfection within specific regions and tissues of interest were homogenized and observed for bioluminescence. Intraperitoneal injections revealed limited penetration of LNP across the BBB with luciferase expression primarily observed in the liver and spleen as expected (FIG. 12). However, intracisternal injections at far lower doses of total encapsulated mRNA successfully transfected brain tissue with subsequent induced protein expression (FIG. 12). As measured by bioluminescence in whole brain homogenate, the bioluminescent signal significantly increased compared to untreated or intraperitoneally injected mice.

In order to analyze the distribution of LNP within the brain, brain regions were resected from WT or P301S Tau mice after intraperitoneal or intracisternal administration. After intraperitoneal injection, some luciferase expression was observed, but primarily in pooled regions of the brain (including the thalamus, hypothalamus and midbrain) at relatively low levels (FIG. 12). In contrast, intracisternal injections predominantly delivered to the brainstem, inducing significant protein expression within this region (FIG. 12).

In some embodiments, the present disclosure describes a carrier system that is capable of delivering RNA therapeutics to a specific cell population in the brain. Specifically, this system explores the usage of lipid nanoparticles, one of the most clinically advanced forms of carrier molecules for RNA therapeutics, to modulate genetic expression and manipulation within microglia. As disease-associated inflammatory microglia actively express ApoE4 proteins, the disclosed LNP system will preferentially target these cells through an ApoE-mediated mechanism at the nanoparticle surface.

Further, from the standpoint of the carrier molecule, structural modifications will be explored at the level of the ionizable lipid and other molecular excipients. Polymeric carriers will also be considered. Moreover, targeting ligands that can be decorated onto the surface of the carrier system will also be explored to improve selectivity for given cell types relative to unmodified carriers.

### Example 2

Formulations were prepared comprising (a) an ionizable amino lipid, (b) a sterol, (c) a phospholipid, and (d) a PEG-lipid in a molar ratio of 35:16:46.5:2.5 and a 10:1 lipid:mRNA weight ratio. Additional formulations modulating the weight ratio of the ionizable lipid:mRNA were also prepared. Various formulations were prepared altering the phospholipid selection. Additional formulations were prepared in which the ionizable lipid was replaced with a phospholipid or cationic lipid. The formulations are summarized in Table 1.

TABLE 1
LNP Formulations

| Formulation | Components | Lipid:mRNA wt ratio |
|---|---|---|
| A | cKK-E12, DOPE, Cholesterol, | 10:1 cKK-E12:mRNA |
| | C14-PEG2000 | |
| B | cKK-E12, DOPE, Cholesterol, | 5:1 cKK-E12:mRNA |
| | C14-PEG2000 | |
| C | DOPE, Cholesterol, | 10:1 DOPE:mRNA |
| | C14-PEG2000 (no cKK-E12) | |
| D | DOPE, Cholesterol, | 5:1 DOPE:mRNA |
| | C14-PEG2000 (no cKK-E12) | |
| E | cKK-E12, DSPE, Cholesterol, | 10:1 cKK-E12:mRNA |
| | C14-PEG2000 | |
| F | cKK-E12, DSPC, Cholesterol, | 10:1 cKK-E12:mRNA |
| | C14-PEG2000 | |
| G | DOTAP, DOPE, Cholesterol, | 10:1 DOTAP:mRNA |
| | C14-PEG2000 | |

### Example 3

### Results and Discussion

Potent In Vitro Delivery of mRNA by LNP is Achieved in Microglia

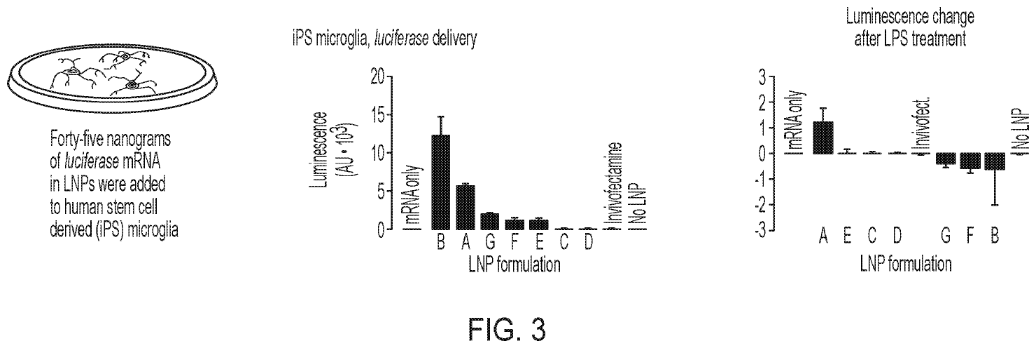

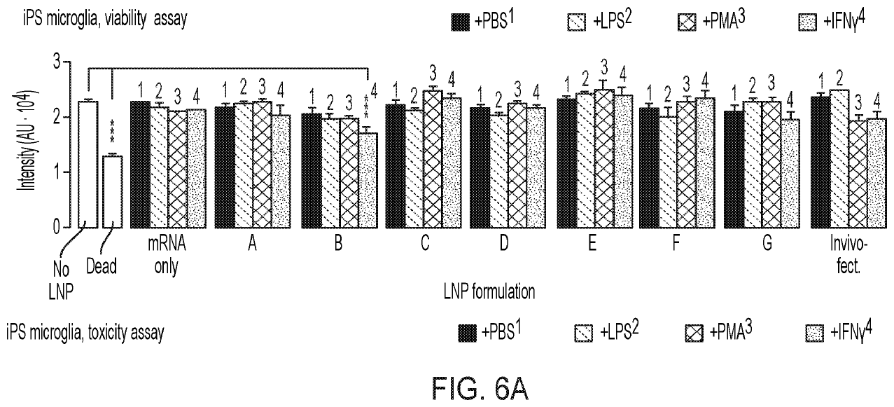

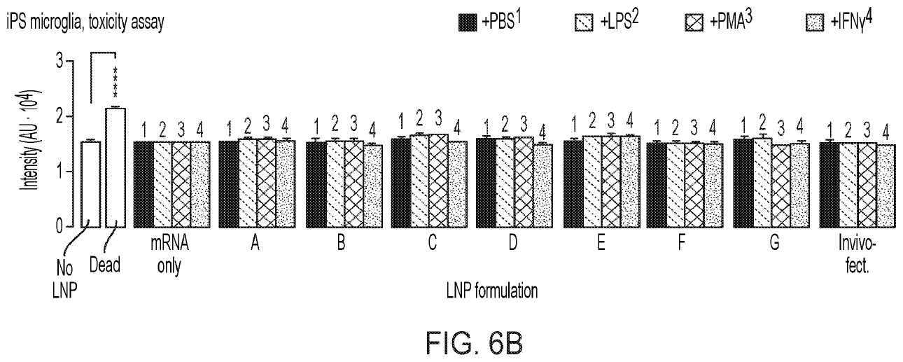

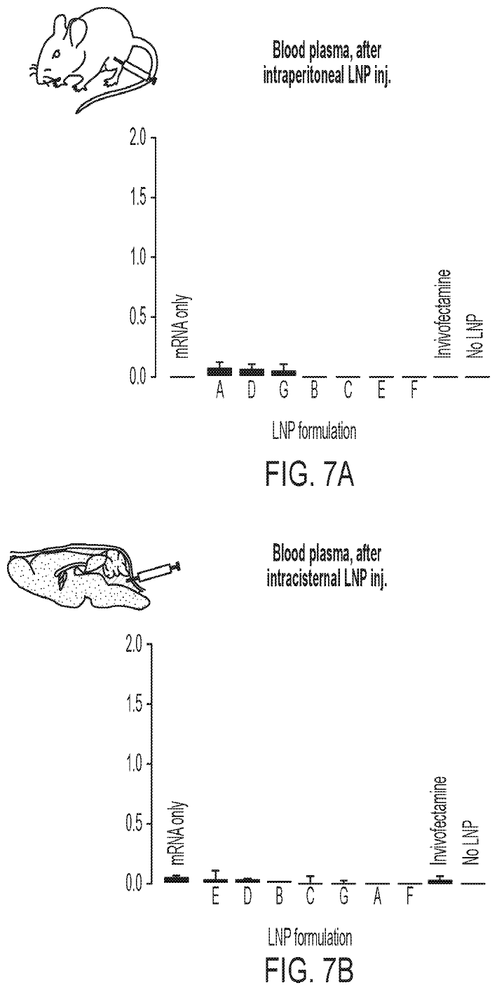

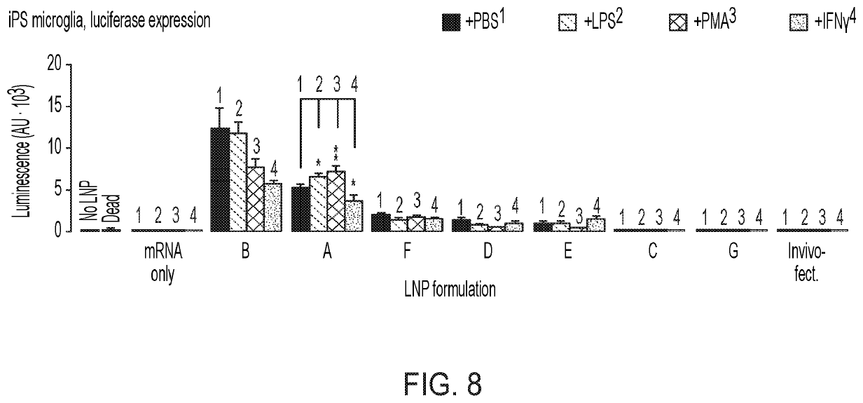

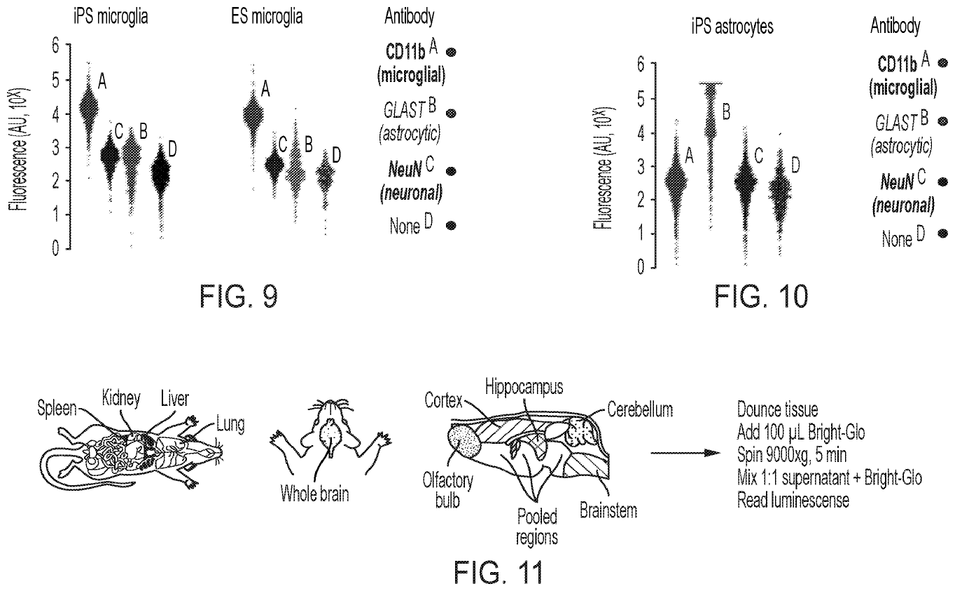

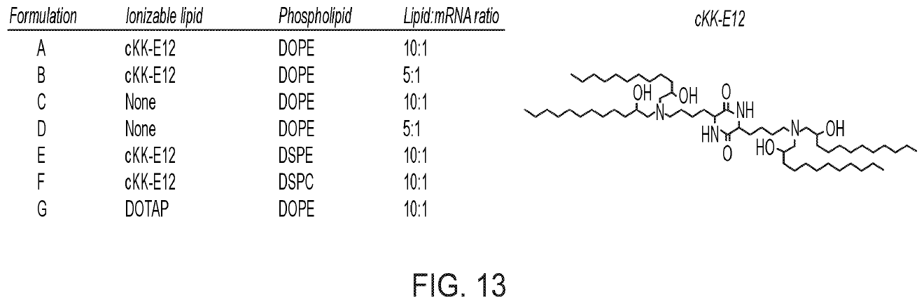

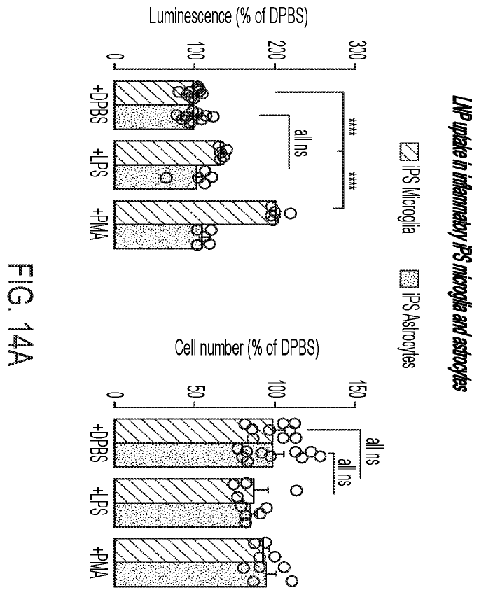

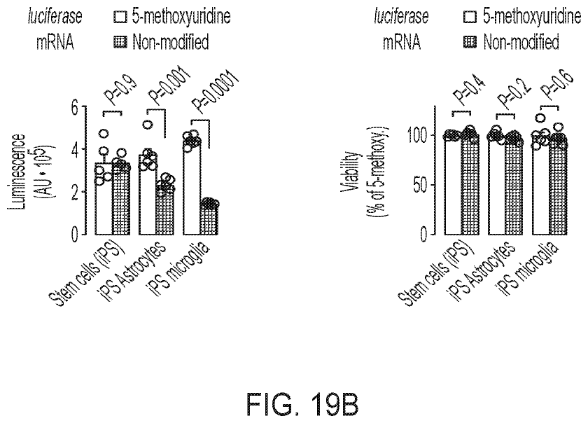

To identify a lead LNP formulation for eventual in vivo delivery, formulations were first screened in vitro in iPS microglia and ES microglia, which have been validated as cellular models of AD.⁴⁰⁻⁴² Microglial differentiation was validated using flow cytometry and immunohistochemistry (IHC) for CD11b, a microglial marker (FIGS. 2B, 2F, 9, and 10). Commercially available transfection reagents are typically less effective in vitro in cells of the brain, with microglia proving especially hard to manipulate, necessitating the need for novel non-viral delivery vectors.⁴³ Thus, a small library of eight LNP candidates were formulated through microfluidic mixing and selected for initial screening.⁷ LNP parameters were modified around a base formulation to focus on the structural identity of the ionizable lipid, phospholipid, or through variation of the lipid:RNA ratios (FIG. 13).⁶ Initial delivery experiments were performed using firefly luciferase (luc) mRNA (Trilink Biosciences) allowing for rapid comparisons of delivery efficacy through quantification of induced luminescent signals. Non-canonical 5-methoxyuridine (5moU) modifications were used in all mRNA sequences as these modulations were shown to significantly increase protein production in microglia, most likely due to improved immune evasion (FIGS. 2B, 2F, 6-10, 19A, and 19B).^{44,45} Of the screened candidates, all LNP containing an ionizable lipid achieved significant levels of luciferase expression in iPS microglia and outperformed commercially available Invivofectamine (ThermoFisher) (FIG. 3).

As delivery to inflammatory microglia is of particular interest for treatment of AD, LNP delivery efficacy was also evaluated after treating the cells with immunostimulatory signals LPS, PMA, and IFNγ. When comparing luciferase expression in iPS microglia with and without inflammatory LPS treatment, only one formulation demonstrated a significant increase in observed luminescence (FIG. 3). Interestingly, this was the original formulation optimized for IV administration and RNA delivery to hepatocytes of the liver.⁶ In fact, several formulations exhibited losses in delivery efficacy in inflammatory conditions (FIGS. 2B, 2F, 6-10, 19A, and 19B). Across all LNP formulations, there were no changes in cell viability compared to untreated control cells as measured by both a cellular viability and toxicity assay (FIGS. 2B, 2F, 6-10, 19A, and 19B). This formulation was then further evaluated in iPS and microglia with a dose-response assay ranging from 1 to 200 ng of encapsulated mRNA in LNP in both iPS and ES microglia cultures. Negligible cell toxicity and loss of viability was observed at doses up to 50 ng and a plateauing of delivery efficacy and protein expression above 10 ng/mL. When analyzing only luminescence, this LNP formulation outperformed a lipofectamine control by nearly an order of magnitude in iPS microglia (FIGS. 2C and 2D).

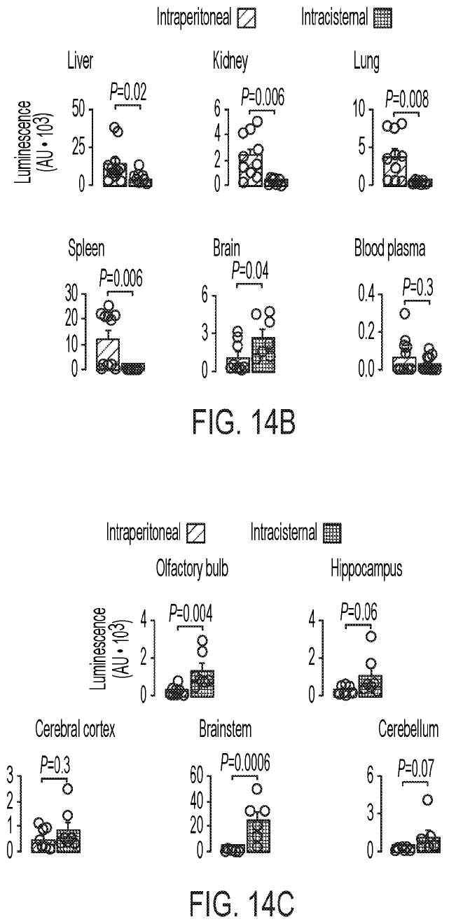

As ApoE uptake pathways in AD are not only misregulated in microglia, but also astrocytes, delivery of LNP in inflammatory astrocytes was evaluated in vitro.¹ Cultured iPS astrocytes were treated with the same immunostimulatory reagents and compared to untreated controls (FIGS. 2B, 2F, 6-10, 19A, and 19B). Upon LNP treatment in the inflammatory microglia culture, a significant increase in luminescence with no corresponding change in the cell number was observed (FIGS. 14A and 2E). When inflammatory iPS astrocytes were treated with LNP, there was no detectable change in the overall level of luminescence nor cell numbers, indicating that there may be a preferential uptake and transfection of inflammatory microglia compared to astrocytes.

### Localized Delivery of LNP In Vivo Efficiently Transfects Cells of the Brain

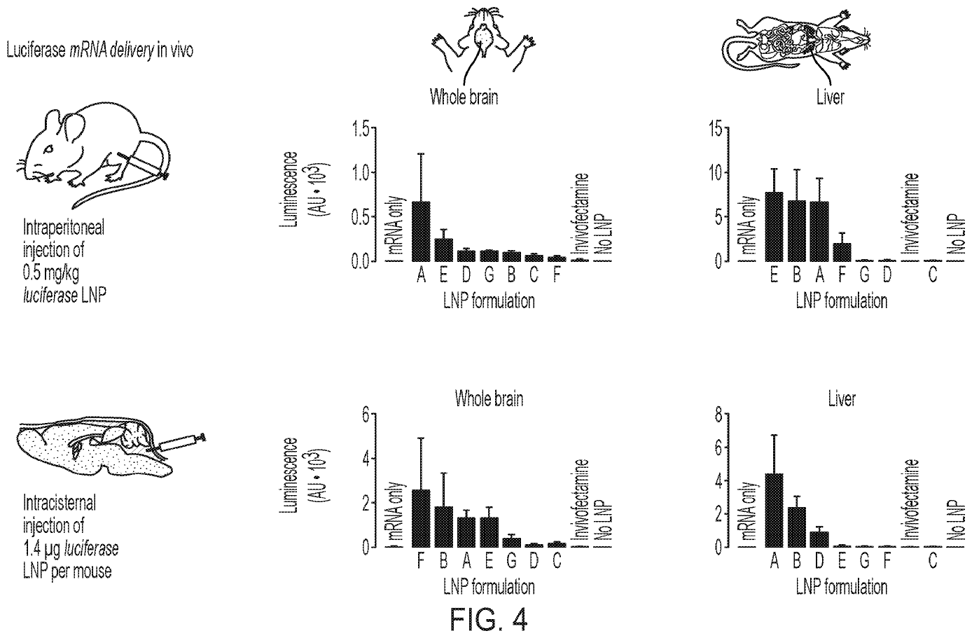

Having established LNP as a valuable tool to achieve mRNA delivery in microglia cell culture, the delivery efficacy was evaluated and the trends in this series of LNP formulations in vivo were compared. Although the base formulation was identified as a potent delivery vehicle administered IV, the large majority (>90%) of protein production is observed within the liver and there is very little penetration across the BBB.^{2,46} With several reports investigating localized delivery of liposomes and polymeric nanoparticles in the brain, intracisternal (IC) injection of LNP to delivery RNA to the brain was selected.^{36,39,47-49} Using the same small library of LNP formulations as were screened in vitro, LNP encapsulating luc mRNA were injected either IP or IC in C57BL/6J mice (FIG. 4). After 24 hours post-injection, the liver and the whole brain were recovered and subsequently homogenized to observe luminescence within these organs. Upon IP injection, several formulations achieved liver transfection. Some luminescence was observed in the brain tissue indicating some passage of the BBB, though at roughly 10%% or less of the total signal seen in the liver. When LNP were locally administered via IC, despite using a far smaller dose (due to volume limitations compared to IP injections), luminescence within the brain was nearly twofold greater (FIG. 4).

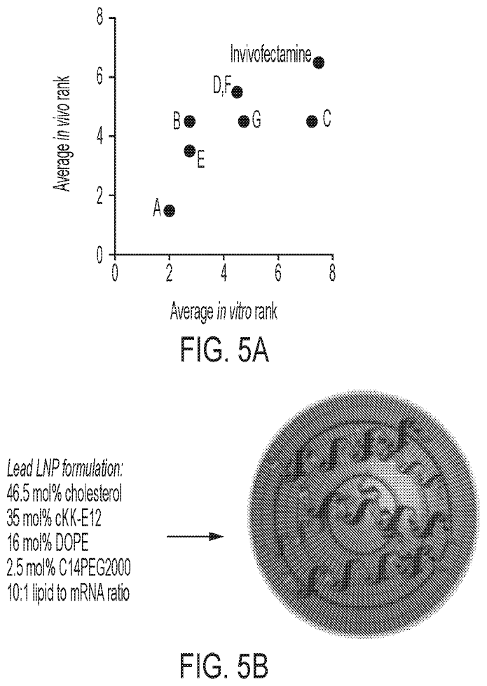

To identify a lead LNP formulation, the overall delivery efficacy both in vitro and in vivo were taken into consideration (FIGS. 5A and 5B). Typically, the correlation of in vitro and in vivo results (IVIVC) is limited in predictive value, necessitating more costly screening experiments in vivo.50,51 However, a strong correlation in IVIVC was observed when comparing the efficacy of LNP in iPS microglia transfection and localized delivery to the brain. Importantly, all LNP formulations outperformed Invivofectamine in delivery to microglia and cells of the brain. In ranking the overall delivery results, formulation A—despite being originally optimized for IV delivery—was the highest performing across replicates both in vitro and in vivo and was therefore selected for future experiments in both mRNA and siRNA delivery.

Intracisternal Administration of mRNA LNP Results in Delivery to the Brainstem

Using IP or IC injections, tissues in proximity to the injection site were then evaluated for LNP-mediated mRNA delivery. Again, individual organs were resected and homogenized to measure the luminescence within the tissues (FIGS. 12, 14B, and 14C). For all internal organs, IP injection was more efficacious, with significant increases in luminescence compared to the IC injections. Luminescent signal was strongest in the liver followed by the spleen. The overall transfection of cells within the blood plasma was negligible in both IP and IC injection (FIGS. 2B, 2F, 6-10, 19A, and 19B). As expected, however, IC injections outperformed IP injection for mRNA delivery in the brain. Brain dissections were performed to evaluate transfection within specific regions of the brain. Here, the effect of the localized IC injection can be observed as the brainstem, the region in closest proximity to the injection site demonstrated significant increases in luminescence, especially compared to regions towards the front of the brain such as the cerebral cortex (FIGS. 12, 14B, and 14C).

Lead LNP Act as a Potent Delivery Vehicle for Anti-PU.1 siRNA In Vitro

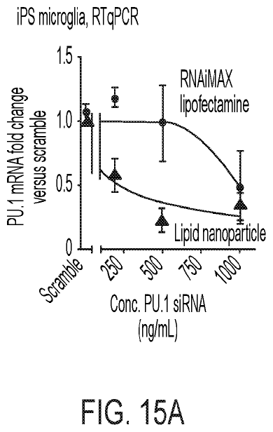

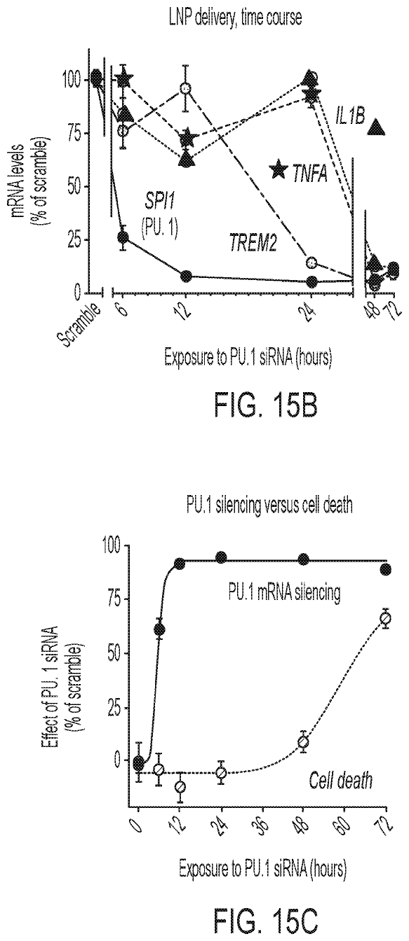

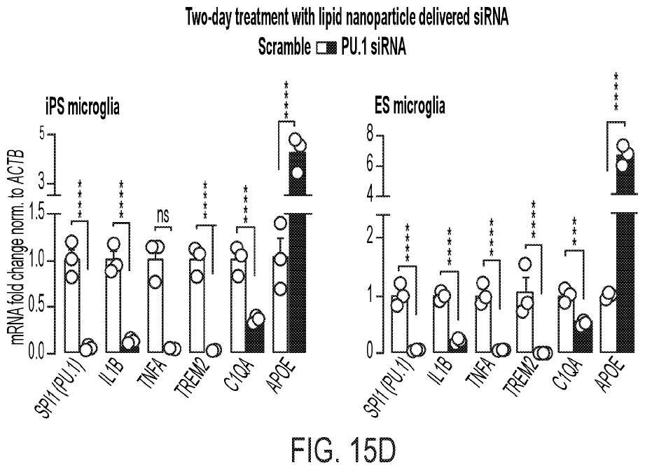

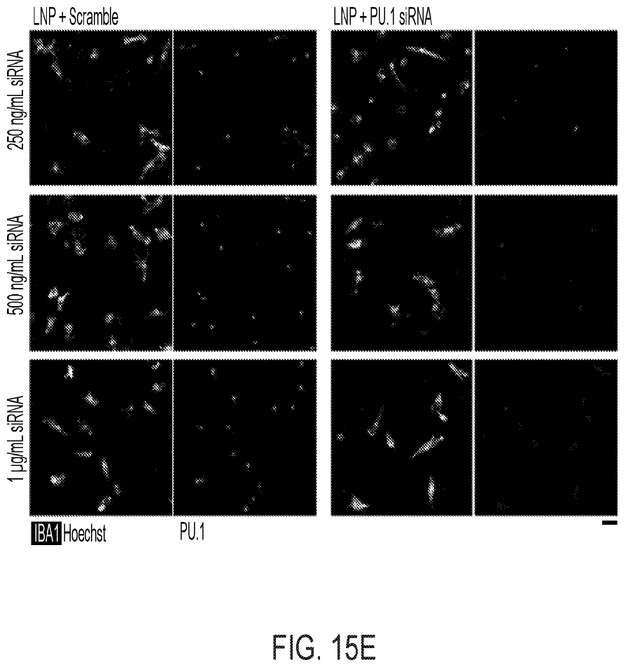

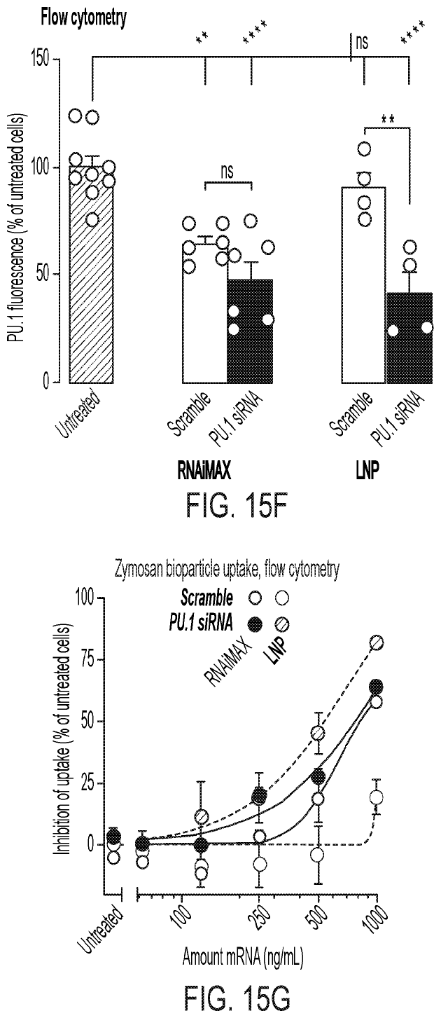

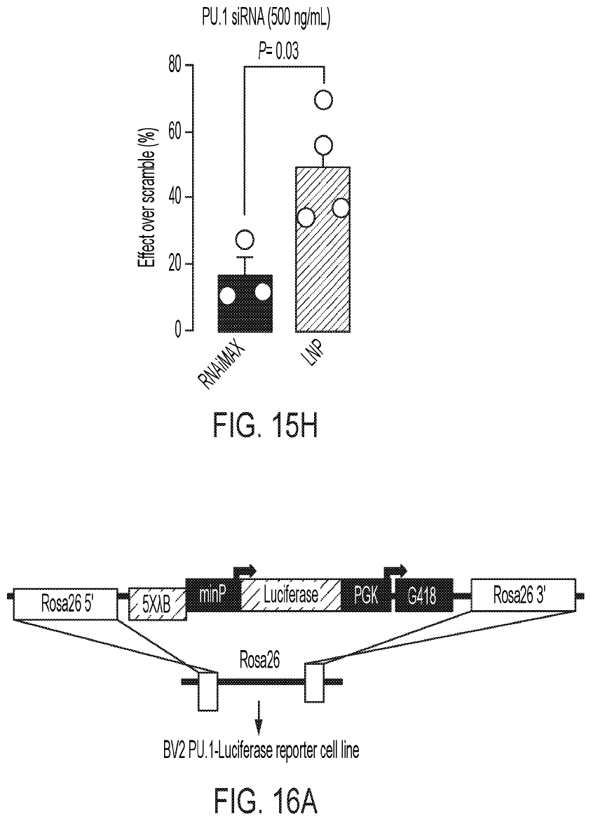

Initial screening efforts of LNP to transfect microglia were performed by encapsulating luc mRNA to use luminescence readings in order to validate successful transfection. However, therapeutic siRNA to address neuroinflammation is of particular interest as a modality to address the causes of neurodegenerative diseases such as AD. Towards this end, LNP formulation A was selected to validate delivery efficacy of anti-PU.1 siRNA in vitro in iPS microglia. When compared to 8 commercially available transfection reagents, LNP were capable of not only producing the greatest knockdown effect (˜50%%), but also maintained the highest overall cell viability (>90%%) (FIGS. 2B, 2F, 6-10, 19A, and 19B). LNP doses of 250 ng/mL of anti-PU.1 siRNA achieved significant silencing of ˜50%% as quantified by RTqPCR. (FIGS. 15A-15C). By comparison, transfection with RNAiMAX lipofectamine required a dose of 1000 ng/mL to achieve similar effects. PU.1 silencing of ˜90% was achieved with siRNA-LNP with negligible change to cell viability after 48 hours, however, silencing led to cell death in iPS microglia culture at the 72-hour timepoint.

Transcript levels of microglial activation markers downstream from PU.1 including TREM2, TNFA, and IL1B were almost completely ablated upon PU.1 knockdown at a 48-hour timepoint (FIG. 15D). When treated with anti-PU.1 siRNA, the levels of APOE transcription rise significantly, further supporting the identified relationship between microglial inflammation and ApoE metabolism pathways. IHC staining of iPS microglia with fluorescent antibodies against PU.1 further demonstrate the knockdown effect of anti-PU.1 siRNA delivered by LNP compared to a scramble siRNA control (FIG. 15E). To validate these results and compare the efficacy of LNP delivery of anti-PU.1 siRNA over cationic transfection reagents, IHC was performed in conjunction with flow cytometry. No appreciable drop in PU.1 fluorescence was observed when scramble siRNA was encapsulated in LNP and a corresponding ˜60%% reduction in fluorescence when microglia were treated with PU.1 siRNA (FIG. 15F). In contrast, RNAimax lipofectamine lowered fluorescence by ˜50%% regardless of the nature of the encapsulated cargo, indicating the knockdown effect occurs through nonspecific means outside of the RNA silencing complex. Using pHrodo-tagged Zymosan A particles to measure levels of particle uptake in a dose-dependent manner, a similar effect is observed. When treated RNAiMax/siRNA complexes, there is only an ˜18%% increase in uptake for PU.1 siRNA versus scramble siRNA controls. In contrast, the increased uptake effect approaches 50%% for LNP delivering anti-PU.1 siRNA (FIGS. 15G and 15H).

LNP Delivery of PU.1 siRNA by Localized Injection has Anti-Inflammatory Effect in Systemic Model of Inflammation

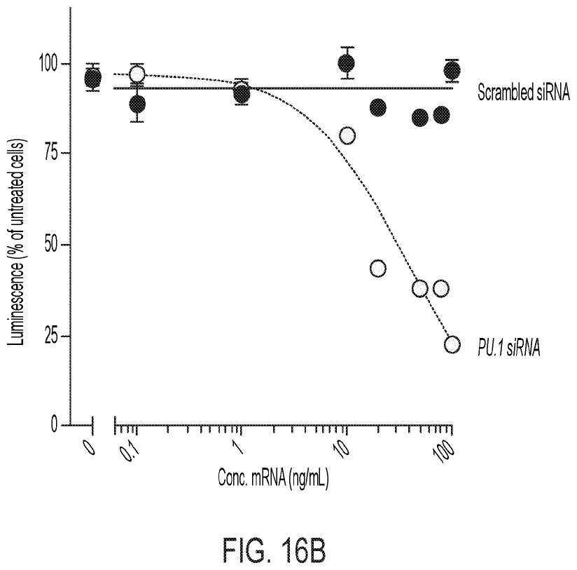

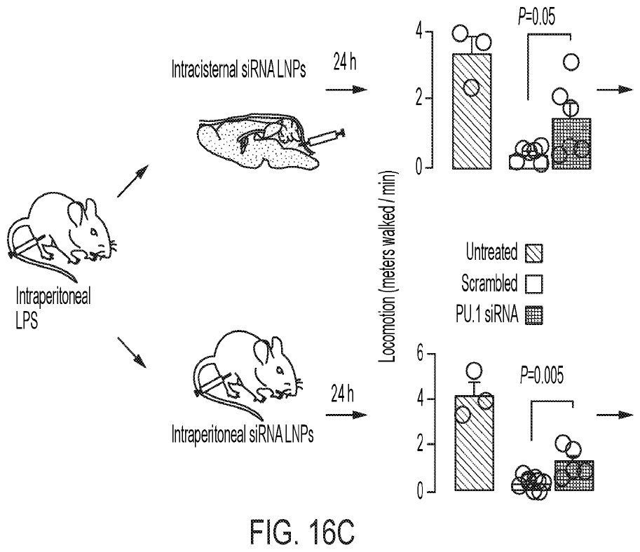

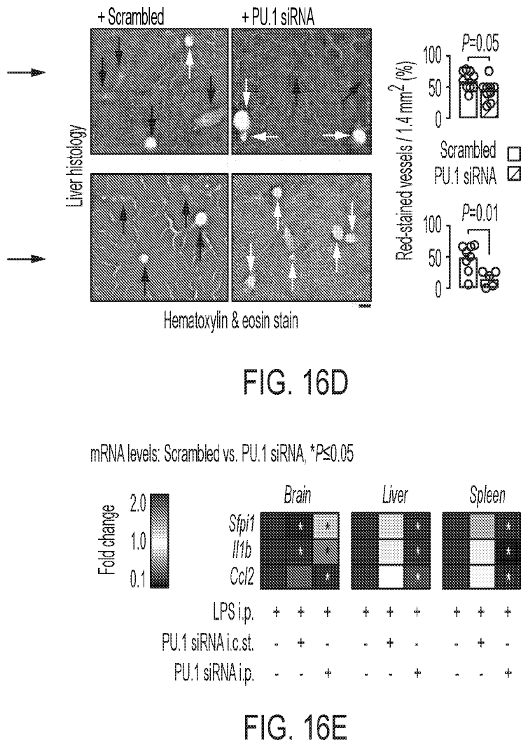

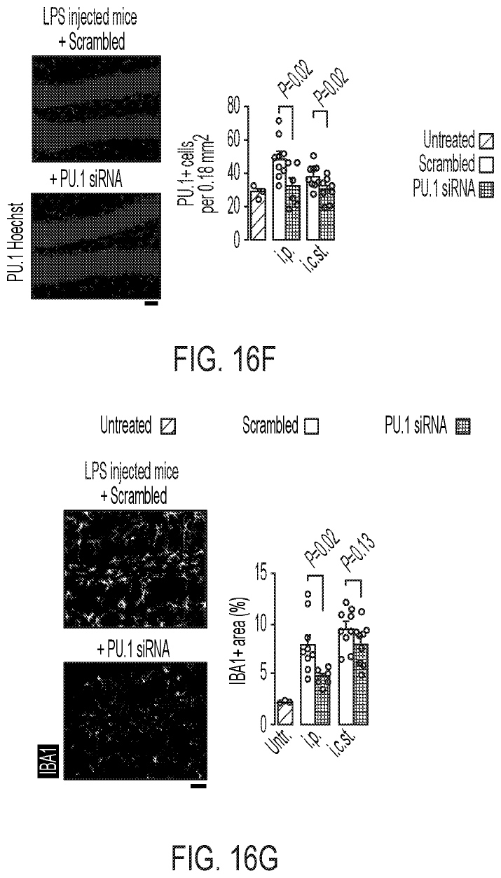

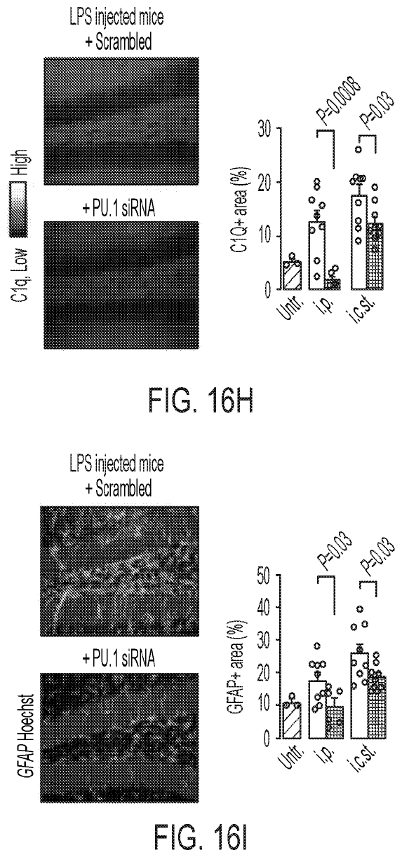

A BV2 mouse microglia cell line that expresses a PU.1-Luciferase reporter construct to help identify compounds with PU.1 knockdown activity through changes in luminescence prior to screening in mouse models was previously developed.^{23,52} BV2-PU.1-Luciferase cells treated with LNP encapsulating scramble siRNA control had no decrease in luminescence (FIGS. 16A and 16B). In BV2-PU.1-Luciferase cells treated with LNP encapsulating anti-PU.1 siRNA (here for the Sfp1 mouse homologue), knockdown was detected at doses as low as 10 ng/mL, with the silencing effect increasing along with siRNA dosing increased up to 100 ng/mL.

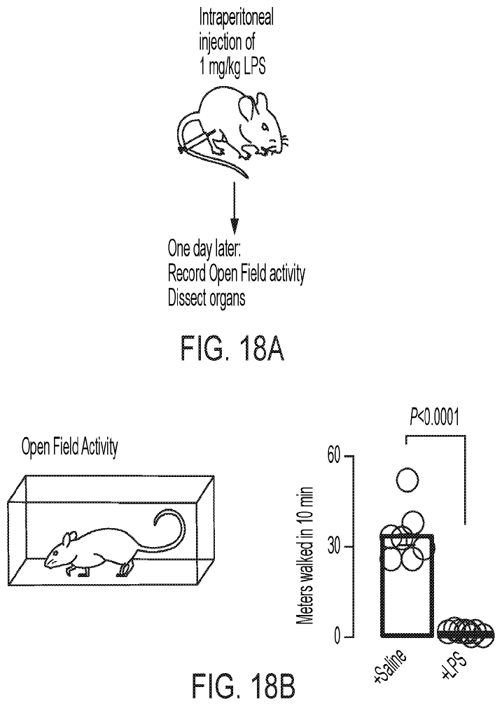

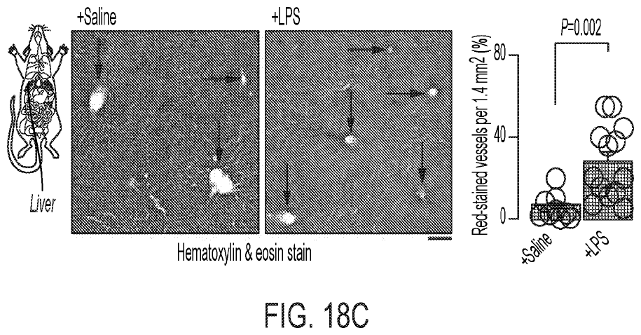

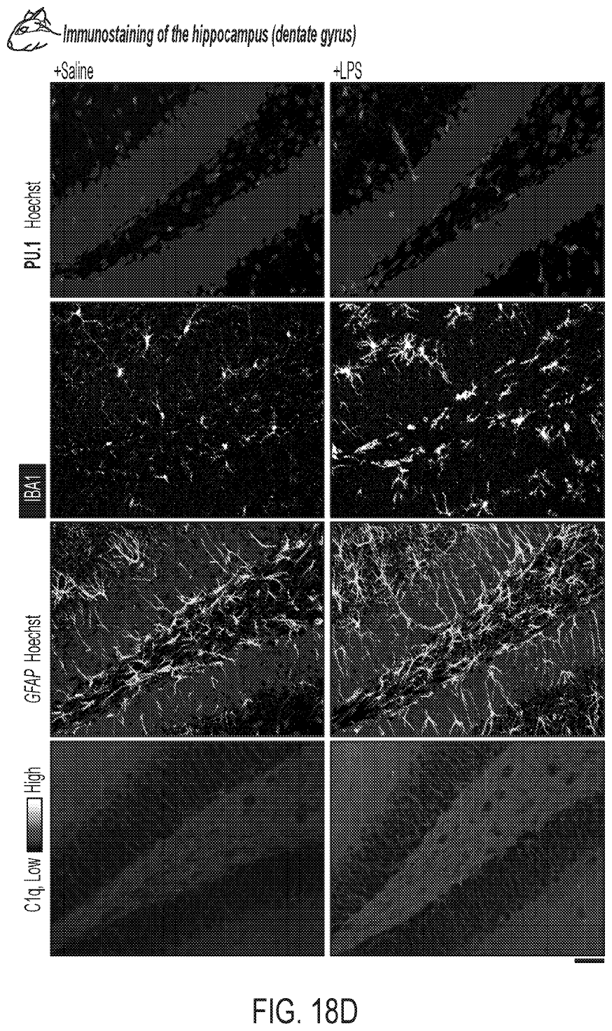

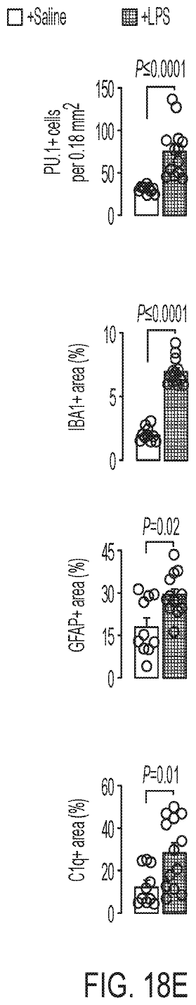

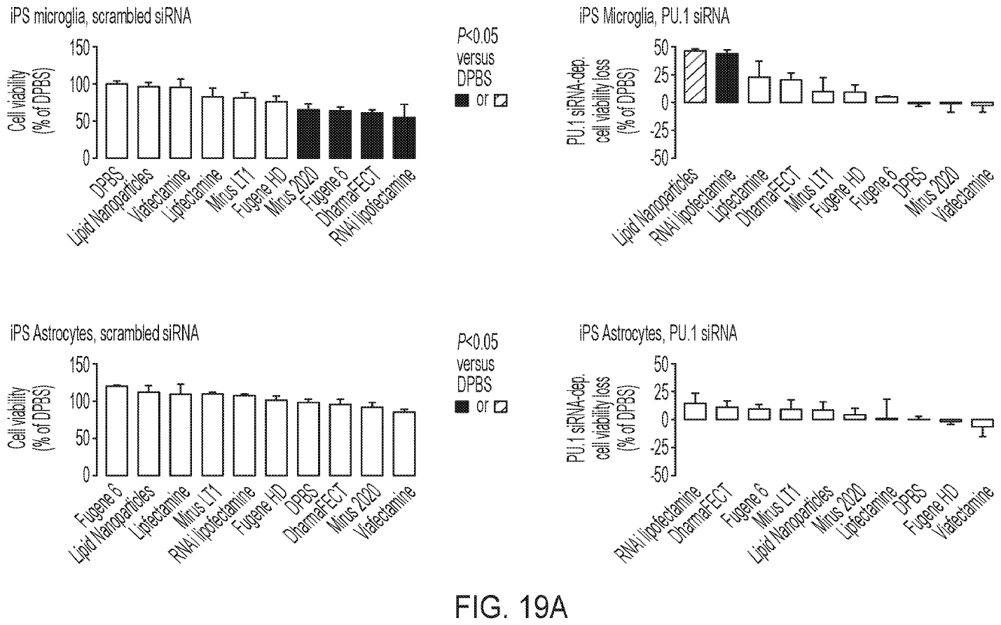

To evaluate the effect of PU.1 knockdown in a mouse model of inflammation, animals were first injected IP with LPS to observe changes in patterns of locomotion and microglial activation markers due to systemic inflammation (FIGS. 18A-18E). Mice were subsequently treated with either IP and IC with PU.1 siRNA-LNP to reduce inflammation. In both treatment groups, animals were observed to cover more distance compared to those treated with control siRNA-LNP, although the effect was more pronounced after IP administration (FIG. 16C). Liver sections were then recovered from treated mice and stained with H&E to look for red-stained vessels indicating the accumulation of immune cells. Again, both IC and IP injections reduced the overall level of red-stained vessels for PU.1 siRNA treatment compared to scramble siRNA (FIG. 16D). IP injections outperformed IC injections supporting the earlier mRNA experiments which demonstrated increased RNA delivery to the liver from IP administration. Additionally, the IP space can tolerate far larger injection volumes which allow for greater dosing of siRNA compared to IC injections.

RTqPCR was performed on resected organs including the brain, liver, and spleen to identify transcript knockdown of Sfp1 and downstream genes IL1B and Ccl2 (FIG. 16E). All genes saw nearly twofold increases in mRNA transcription due to inflammation induced by LPS injection. Both IP and IC injections achieved significant knockdown of PU.1, with IC injections reaching a threshold of 90%% reduction in mRNA transcripts. IC injections did not result in a knockdown effect in internal organs such as the liver and spleen while IP injections were able to reduce transcript levels. Confocal microscopy of hippocampal slices to analyze staining of PU.1-positive nuclei indicated a comparable knockdown effect in both IP and IC administration which restored levels of PU.1-positive cells back to the untreated, non-inflammatory baseline (FIG. 16F). Similarly, IHC staining was performed to quantify microglial activation markers such as including IBA1, C1Q, and GFAP downstream of PU.1 expression. IP injection with PU.1 siRNA-LNP significantly reduced the total fluorescent area compared to scramble siRNA-LNP delivery in all instances. IC injection also reduced the positively-stained area for all cases, though statistical significance was only reached in C1Q and GFAP (FIGS. 16G-16I).

PU.1 siRNA can Reduce Chronic Neuroinflammation

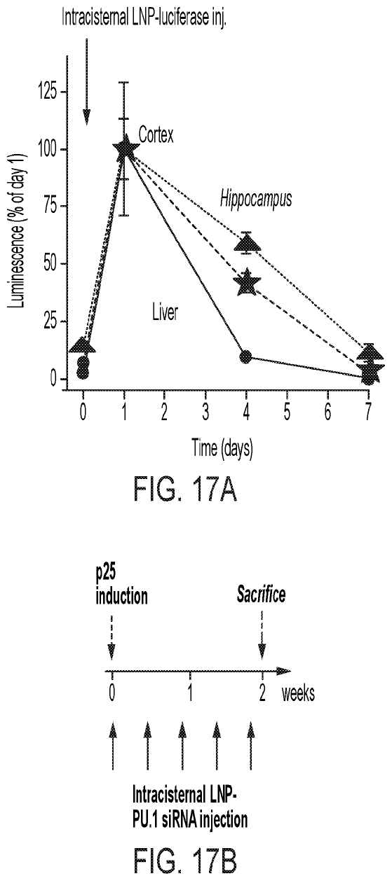

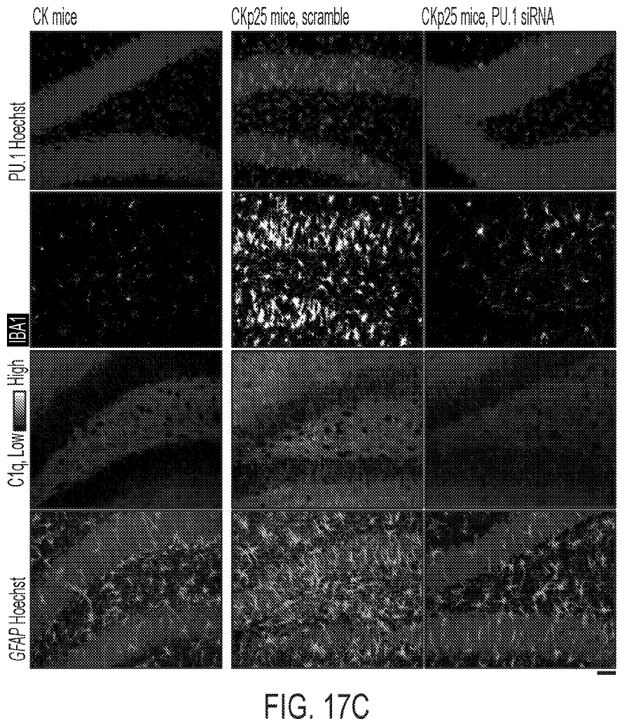

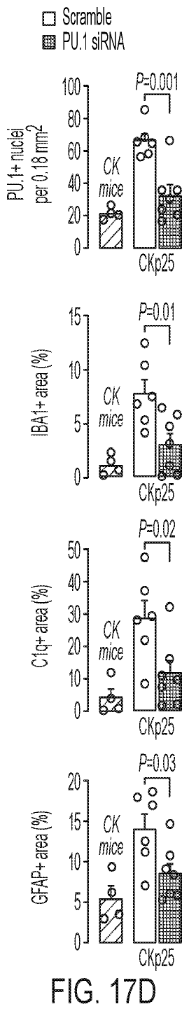

Having demonstrated that anti-PU.1 siRNA treatment effectively reduced levels of microglial activation as a result of acute systemic inflammation, this treatment was evaluated in a specific neuroinflammation context. Anticipating the need for repeated dosing to prolong the anti-inflammatory effect, luc mRNA was used to approximate the timecourse as a result of IC LNP delivery. Luminescence faded completely by day 7 with detected signal dropping ˜50%% by day 4 post-injection (FIG. 17A).

To investigate localized IC LNP delivery in a central inflammation context, the CKp25 mouse model of AD was used. Animals were induced through p25 injection for two weeks and treated every 72 hours with LNP encapsulating scramble or anti-PU.1 siRNA (Days 0, 3, 6, 9, 12) (FIG. 17B). After 14 days, animals were sacrificed and hippocampal tissue was stained through IHC for PU.1 and microglial activation markers. Anti-PU.1 siRNA again significantly reduced levels of PU. 1 expression compared to the treatment with control scramble siRNA (FIGS. 17C-17D). Upon repeated dosing with PU.1 LNP, the presence of PU.1-positive cells was nearly reduced to baseline control levels. Downstream activation markers IBA1, C1q, and GFAP were again reduced in hippocampal tissue compared to treatment with scramble siRNA. Taken together, these results indicate that PU.1 siRNA can be used as an anti-inflammatory therapeutic to address underlying neuroinflammation in the Ckp25 AD model.

### CONCLUSIONS

LNP were used as a potent RNA delivery vehicle within microglia and cells of the brain, demonstrating the role of PU.1 as a potential target for antiinflammatory siRNA therapeutics. Through the screening of a small sequence of LNP design parameters, a formulation was identified which resulted in preferential delivery to inflammatory microglia. Using the LNP to deliver luc mRNA, potent transfection in iPS microglia was observed and luminescence was detected in all regions of the brains, especially the brainstem as the site of injection. This same LNP formulation was capable of delivering PU.1 siRNA far more effectively than commercial reagents both in vitro and in vivo. The resulting knockdown of PU.1 displayed an antiinflammatory effect in systemic inflammation from LPS administration, lowering expression levels of PU.1 as well as downstream microglial activation markers. Finally, in a CKp25 model of AD neuroinflammation, anti-PU.1 siRNA delivered repeatedly via LNP to the IC space had a significant antiinflammatory effect. Anti-PU.1 siRNA-LNP can therefore act as a powerful delivery tool to treat AD and other diseases with underlying microglial inflammation.

More broadly, LNP demonstrate potent delivery of RNA to screen the effects of genetic perturbations on microglia culture in vitro to identify novel therapeutic candidates. This project focused on a limited swath of space in LNP design and additional formulation optimization may reveal LNP candidates which significantly improve delivery efficacy. Future works focused on the expansion of this LNP platform with modifications which may facilitate crossing of the BBB or to extend the longevity of the knockdown effect and reduce dosing protocols could also realize the potential synergy of LNP-mediated delivery of therapeutic RNA to identify treatments which may prevent or cure AD.

### Methods

### Human iPS and ES Microglia Experiments

Unedited human iPS (Coriell, AG09173) and ES (WiCell, WA09) cells were cultured on plates (VWR, 62406-161) coated with Matrigel (VWR, BD354277) in mTeSR1 media (STEMCELL, 85850), and lifted with ReLeSR (STEMCELL, 05872). G-band karyotyping was performed by Cell Line Genetics. iPS and ES cells were differentiated into microglia using an optimized version of a published protocol,⁸ starting with the STEMdiff Hematopoietic Kit (STEMCELL, 05310) reagents for 12 days, followed by maintenance in DMEM/F12, HEPES, 2% ITS-G (ThermoFisher, 41400045), 2% B27 (ThermoFisher, 17504044), 0.5% N2, 1% Glutamax (ThermoFisher, 17502048), 1% NEAA, 1% PenStrep, 100 μM β-mercaptoethanol (Millipore, M6250), 25 ng/mL MCSF (PeproTech, 200-35) and 100 ng/mL IL34 (PeproTech, 200-34). The cells were used between days 45 and 200 in culture. Purity of microglia cultures was assessed via flow cytometry (BD, FACSaria IIu) after washes in DPBS, blocking in FcR blocking reagent (Miltenyi, 130-059-901), and labelling with CD11b-APC or NeuN-488. Before flow cytometry, the cells were lifted using cell scrapers (Corning, 353085) and passed through a 35 μm filter cap tube (ThermoFisher, 08-771-23). Immunocytochemistry was performed on cells plated directly on untreated glass slides (Millipore, PEZGS0816) followed by 4% paraformaldehyde fixation and a one-hour incubation in blocking solution, consisting of 5% Normal Donkey Serum (Millipore, S30-M) and 0.3% Triton-X (Millipore, T8787) in DPBS. Cells were incubated with primary antibody overnight in blocking solution, followed by three 10-minute DPBS washes before exposure to a secondary antibody for 30 minutes at room temperature, and then three 10-minute DPBS washes with 1 μg/mL Hoechst (ThermoFisher, H3570) added to the last wash. Finally, the glass coverslip was mounted using Fluoromount-G (VWR, 100502-406) and left to solidify overnight at room temperature. Confocal microscopy (Zeiss, LSM 880) was performed using a 20× Plan-Apochromat objective (Zeiss, 421452-9880-000). Luminescence was triggered with Bright-Glo reagents (Promega, E2650) and immediately measured using an EnVision Multilabel Plate Reader (PerkinElmer, 2105-0010). Toxicity and loss of viability was measured with MultiTox-Fluor Multiplex Cytotoxicity Assay (Promega, G9200), and read on the EnVision Multilabel Plate Reader, after 30 minutes incubation at RT. Uptake assays were performed by overnight incubation of stem cell-derived microglia in clear-bottom 96 well plates (VWR, 3603) with 30 μg/mL pHrodo-labelled zymosan A bioparticles (ThermoFisher, P35365), analyzed with flow cytometry (BD, FACS Celesta HTS-1). Microglial activation was achieved by a two-day incubation with 25 ng/mL of either IFNγ (R&D Systems, 285-IF-100), LPS (Millipore, L-2654), or PMA (Millipore, P1585-25MG). Commercially used transfection reagents were added exactly according to the instructions of the manufacturer (Lipofectamine: ThermoFisher, 18324012; Lipofectamine RNAiMAX: ThermoFisher, 13778075; Invivofectamine, ThermoFisher, IVF3001). Astrocytes were derived from human iPS (Coriell, AG09173) cells.⁹

### BV2 PU.1 Reporter Cell Line

The BV2 PU.1-Luciferase reporter cell line was made by inserting five tandem copies of the λB motif,¹⁰ followed by a minimal promoter and the luciferase coding sequence from the pGL4.23 luciferase plasmid (Promega, E8411), into the pROSA26-1 plasmid (Addgene, 21714). Then, the reporter construct was transfected into BV2 cells with lipofectamine (ThermoFisher, 18324012). BV2 cells were cultured in RPMI media (Millipore, R7388-500ML), 1% PenStrep (Wisent, 450-201-EL), 10% FBS (Gemini, 100-106) and were under constant selection with 1% G418 (ThermoFisher, 10131027), grown in CELLSTAR flasks (VWR, 82050-872), and passaged with TrypLE (ThermoFisher, 12605028).

### Lipid Nanoparticle Preparation

LNP were formulated through a microfluidic mixing process as previously described.^{7,4} Ionizable lipid cKK-E12 was synthesized according to previously published protocols.² Cholesterol, phospholipids, and PEG-lipid were purchased from Avanti Polar Lipids and stored at −20° C. prior to use. Briefly, all lipids were dissolved as stock solutions in 100% ethanol at a concentration of 10-20 mg/mL. RNA was dissolved in 10 mM citrate buffer pH 3.0 at a concentration of 133 ug/mL. Lipid solutions were mixed with a 35:46.5:16:2.5 mol % ratio of ionizable lipid:cholesterol:phospholipid:PEG-lipid unless otherwise specified. The final lipid solution was calculated such that the final ratio of ionizable lipid:RNA was 10:1 unless otherwise indicated. These solutions were mixed through dual syringe pumps at a volume ratio of 3:1 aqueous:ethanol within a microfluidic channel in a PDMS mold, resulting in LNP with a final RNA concentration of 100 ug/mL. LNP solutions were then dialyzed against 1×PBS using 20K MWCO (for mRNA) or 3.5K MWCO (siRNA) Slide-a-lyzer cassettes (ThermoFisher®) at 4° C. for at least 2 hours. After dialysis, LNP aliquots were removed for characterization and the remaining solution was stored at 4° C. until dosing experiments were performed in vitro or in vivo. For in vivo experiments, LNP were concentrated using centrifugal filters of the appropriate MWCO (20K for mRNA, 3.5K for siRNA LNP) at 2000 rcf for 20 minutes at 4° C.

### Mouse Experiments

All of the experiments with mice were approved by the Committee for Animal Care of the Division of Comparative Medicine at Massachusetts Institute of Technology. Mice were housed on a 12-hour light-dark cycle at 24° C., 45% relative humidity and food (Envigo, Teklad RMH 3000) and water accessible ad libitum. Experiments were performed in male, two-month-old C57BL/6J (JAX, 000664) mice. Lipopolysaccharides (Millipore, L6529) were injected using a 1 mL syringe (BD, 309659) with a 26G needle (BD, 305110) at 10 mg/kg one day before behavioral experiments and perfusions. Intracisternal injection was performed with a 30G needle (BD, 305128). The cap of the needle was cut 7 mm from the top, so that 2.5 mm of the needle protrudes beyond the opening. The needle and cut cap was attached to a Luer lock syringe (Hamilton, 81020). Immediately prior to injection, the back of the head is shaved with an electric razor in 2% isoflurane anesthetized mice. The area is then sterilized with 3 alternations of 70% ethanol and povidine. The needle insertion point (foramen magnum) is readily recognizable as a fold in the skin when the head is angled down. Ophthalmic ointment (Dechra, Puralube) was applied to both eyes. Neurodegeneration is in CKp25 mice¹¹ (and control littermate CK mice). In the CKp25 mice, p25 expression is driven by the CaMKII-tTA (CK) promoter crossed to tetO-p25 mice (JAX, 005706) in the absence of doxycycline, with CK mice as littermate controls (JAX, 003010), thus suppressing p25 expression by the 1 g/kg doxycycline diet (Bio-Rad, custom-made) and inducing p25 when mice are put on regular diet. Open Field activity was recorded using a camera attached to the ceiling and analyzed automatically using EthoVision XT (Noldus) software. Open Field activity was averaged from two ten-minute trials measuring the movement of the mouse in a custom-made plastic compartment measuring 40×40 cm. For luciferase mRNA distribution, the mice were decapitated and blood was collected from the neck, followed by collecting internal organs and dissected the brain. The biopsies were then dounce-homogenized with a loose pestle (VWR, 62400-595) in BrightGlo, spun at 15000 rpm, 5 min. The supernatant was then transferred into a white 96-well plate (VWR, 354651) and measured for luminescence using an EnVision Multilabel Plate Reader (PerkinElmer, 2105-0010).

### Reverse Transcriptase Quantitative Polymerase Chain Reaction (RT-qPCR)

mRNA extraction was performed with the RNeasy Plus Mini Kit (Qiagen, 74134) and converted into cDNA using the EcoDry RT PreMix reagent (Takara, 639542). cDNA was added to the SsoFast EvaGreen qPCR mastermix (Bio-Rad, 1725202) and primers were purchased from IDT, plated in Hard-Shell 96-well plates (Bio-Rad, Hsp9641), sealed (Bio-Rad, MSB1001), spun in a PCR plate spinner (Labnet, C1000) and, subsequently, gene expression was quantified using a qPCR detection system (Bio-Rad, CFX96) for 40 cycles. Relative gene expression changes were calculated using the 2^{-ΔΔCT} method. Values were normalized to expression levels of actb.

### Immunohistology in Mice

Mice were deeply anesthetized with 5% isoflurane and intracardially perfused with 4° C. PBS, followed by dissection of the brain and/or peripheral organs, and subsequent drop-fixing in 4% paraformaldehyde overnight at 4° C., followed by sectioning into 40 μm coronal free-floating slices on a vibratome (Leica, VT1200S). Free-floating sections were blocked in blocking solution (5% Normal Donkey Serum (Millipore, S30-M) and 0.3% Triton-X (Millipore, T8787)) and stained overnight in blocking solution with primary antibody, at 4° C. Secondary antibody was added in blocking solution for 30 minutes at room temperature, followed by three 10-minute DPBS washes with 1 μg/mL Hoechst (ThermoFisher, H3570) added to the last wash. Finally, the glass coverslip was mounted using Fluoromount-G (VWR, 100502-406) and left to solidify overnight at room temperature. Confocal microscopy (Zeiss, LSM 880) was performed using a 20X Plan-Apochromat objective (Zeiss, 421452-9880-000). Livers were stained with Hematoxylin and eosin Y (Tyr Scientific LLC) in the following sequence: sectioning, mounting on glass slide, dehydration in 100% xylene, 100% ethanol, 70% ethanol—followed by rehydration in deionized water and exposure to Hematoxylin, bluing solution, 100% ethanol, eosin Y, 100% ethanol and finally 100% xylene before coverslipping and imaging with a brightfield camera (Zeiss, LSM 900).

TABLE 2

| | Source | Identifier |
|---|---|---|
| Product | | |
| Anti-human CD11b-APC, 1:50 | Miltenyi | 130-113-231 |
| Anti-human/mouse IBA1, 1:500 | SYSY | 234004 |
| Anti-human PU.1, 1:500 | CST | 2258S |
| Anti-human PU.1, 1:500 | CST | 2266S |
| Anti-mouse GFAP, 1:500 | Abcam | Ab53554 |
| Anti-mouse C1q, 1:500 | Abcam | ab 182451 |
| CellTiter-Glo | Promega | G7570 |
| Software | | |
| ImageJ | NIH | https://imagej.net/Welcome |
| Prism | GraphPad | https://www.graphpad.com |
| EthoVisionXT | Noldus | https://www.noldus.com/ethovision-xt |
| Cyberduck | Open source | https://cyberduck.io/ |

TABLE 3
RTqPCR primers.

| | | |
|---|---|---|
| TREM2-F | Human | ATGATGCGGGTCTCTACCAG |
| TREM2-R | Human | GGAACCAGAGATCTCCAGCA |
| IL1B-F | Human | GCATCCAGCTACGAATCTCC |
| IL1B-R | Human | TCGTTATCCCATGTGTCGAA |
| APOE-F | Human | CGCTTTTGGGATTACCTGCG |
| APOE-R | Human | GGGGTCAGTTGTTCCTCCAG |
| β ACTIN-F | Human | GGATGCAGAAGGAGATCACTG |
| β ACTIN-R | Human | CGATCCACACGGAGTACTTG |
| PU.1-F | Human | GCGTGCAAAATGGAAGGGTT |
| PU.1-R | Human | AGATCCGTGTCATAGGGCAC |
| TYROBP-F | Human | CCTGGCCGTGTACTTCCTG |
| TYROBP-R | Human | TCGCTGTAGACATCCGACCT |
| CCL2-F | Human | TCCCAAAGAAGCTGTGATCTTCA |
| CCL2-R | Human | TTTGCTTGTCCAGGTGGTCC |
| C1Q-F | Human | GCATCCAGTTGGAGTTGACA |
| C1Q-R | Human | GTCCTCGGTCACCATAGAGG |

### REFERENCES

1. Sienski, G. et al. APOE4 disrupts intracellular lipid homeostasis in human iPSC-derived glia. Sci. Transl. Med. 13, (2021).
2. Dong, Y et al. Lipopeptide nanoparticles for potent and selective siRNA delivery in rodents and nonhuman primates. Proc. Natl. Acad. Sci. 111, 3955-3960 (2014).
3. Fenton, O. S. et al. Bioinspired Alkenyl Amino Alcohol Ionizable Lipid Materials for Highly Potent in Vivo mRNA Delivery. Adv. Mater. 28, 2939-2943 (2016).
4. Fenton, O. S. et al. Customizable Lipid Nanoparticle Materials for the Delivery of siRNAs and mRNAs. Angew. Chemie Int. Ed. 57, 13582-13586 (2018).
5. Fenton, O. S. et al. Synthesis and Biological Evaluation of Ionizable Lipid Materials for the In Vivo Delivery of Messenger RNA to B Lymphocytes. Adv. Mater. 29, 1606944 (2017).
6. Kauffman, K. J. et al. Optimization of Lipid Nanoparticle Formulations for mRNA Delivery in Vivo with Fractional Factorial and Definitive Screening Designs. Nano Lett. 15, 7300-7306 (2015).
7. Chen, D. et al. Rapid Discovery of Potent siRNA-Containing Lipid Nanoparticles Enabled by Controlled Microfluidic Formulation. J. Am. Chem. Soc. 134, 6948-6951 (2012).
8. McQuade, A, M Coburn, CH Tu, J Hasselmann, H Davtyan and Blurton-Jones M. 2018. Development and validation of a simplified method to generate human microglia from pluripotent stem cells. Mol Neurodegener 13(1)67.
9. Lin, Y T, J Seo, F Gao, H M Feldman, H L Wen, J Penney, H P Cam, E Gjoneska, W K Raja, J Cheng, R Rueda, O Kritskiy, F Abdurrob, Z Peng, B Milo, C J Yu, S Elmsaouri, D Dey, T Ko, B A Yankner and L H Tsai. 2018. APOE4 Causes Widespread Molecular and Cellular Alterations Associated with Alzheimer's Disease Phenotypes in Human iPSC-Derived Brain Cell Types. Neuron 98(6):1294.
10. Munde, M, S Wang, A Kumar, C E Stephens, A A Farahat, D W Boykin, W D Wilson and G M Poon. 2014. Structure-dependent inhibition of the ETS-family transcription factor PU.1 by novel heterocyclic diamidines. Nucleic Acids Res. 42(2)1379-90.
11. Cruz, J C, HC Tseng, J A Goldman, H Shih and L H Tsai. 2003. Aberrant Cdk5 activation by p25 triggers pathological events leading to neurodegeneration and neurofibrillary tangles. Neuron 40(3):471-83.
12. Zhu, A, J G Ibrahim and M I Love. 2019. Heavy-tailed prior distributions for sequence count data: removing the noise and preserving large differences. Bioinforma. 35(12)2084-2092.
13. Chen, W. W., Zhang, X. & Huang, W. J. Role of neuroinflammation in neurodegenerative diseases (Review). Mol. Med. Rep. 13, 3391-3396 (2016).
14. Kwon, H. S. & Koh, S. H. Neuroinflammation in neurodegenerative disorders: the roles of microglia and astrocytes. Translational Neurodegeneration vol. 9 1-12 (2020).
15. Amor, S., Puentes, F., Baker, D. & Van Der Valk, P. Inflammation in neurodegenerative diseases. Immunology vol. 129 154-169 (2010).
16. Knowles, T. P. J., Vendruscolo, M. & Dobson, C. M. The amyloid state and its association with protein misfolding diseases. Nature Reviews Molecular Cell Biology vol. 15 384-396 (2014).
17. Deverman, B. E., Ravina, B. M., Bankiewicz, K. S., Paul, S. M. & Sah, D. W. Y. Gene therapy for neurological disorders: progress and prospects. Nat. Rev. Drug Discov. 17, 641-659 (2018).
18. Hickman, S., Izzy, S., Sen, P., Morsett, L. & El Khoury, J. Microglia in neurodegeneration. Nature Neuroscience vol. 21 1359-1369 (2018).
19. Smith, A. M. et al. The transcription factor PU.1 is critical for viability and function of human brain microglia. Glia 61, 929-942 (2013).
20. Pimenova, A. A. et al. Alzheimer's-associated PU.1 expression levels regulate microglial inflammatory response. Neurobiol. Dis. 148, 105217 (2021).
21. Gjoneska, E. et al. Conserved epigenomic signals in mice and humans reveal immune basis of Alzheimer's disease. Nature 518, 365-369 (2015).
22. Drager, N. M. et al. A CRISPRi/a platform in human iPSC-derived microglia uncovers regulators of disease states. Nat. Neurosci. 25, 1149-1162 (2022).
23. Ralvenius, W. T., Tsai, L., Woolf, H., Cam, H. & Mungenast, A. A novel PU.1 inhibitor for the treatment of Alzheimer's disease. Alzheimer's Dement. 17, (2021).
24. Holtzman, D. M., Herz, J. & Bu, G. Apolipoprotein E and apolipoprotein E receptors: normal biology and roles in Alzheimer disease. Cold Spring Harb. Perspect. Med. 2, a006312 (2012).
25. Lin, Y. T. et al. APOE4 Causes Widespread Molecular and Cellular Alterations Associated with Alzheimer's Disease Phenotypes in Human iPSC-Derived Brain Cell Types. Neuron 98, 1141-1154.e7 (2018).
26. Pardridge, W. M. Blood-Brain Barrier and Delivery of Protein and Gene Therapeutics to Brain. Front. Aging Neurosci. 11, 373 (2020).
27. Dowdy, S. F. Overcoming cellular barriers for RNA therapeutics. Nature Biotechnology vol. 35 222-229 (2017).
28. Andresen, J. L. & Fenton, O. S. Nucleic acid delivery and nanoparticle design for COVID vaccines. MRS Bull. 46, 1-8 (2021).
29. Polack, F. P. et al. Safety and Efficacy of the BNT162b2 mRNA Covid-19 Vaccine. N. Engl. J. Med. 383, 2603-2615 (2020).
30. Baden, L. R. et al. Efficacy and Safety of the mRNA-1273 SARS-CoV-2 Vaccine. N. Engl. J. Med. 384, 403-416 (2021).
31. Lorenzer, C., Dirin, M., Winkler, A. M., Baumann, V. & Winkler, J. Going beyond the liver: Progress and challenges of targeted delivery of siRNA therapeutics. Journal of Controlled Release vol. 203 1-15 (2015).
32. Zhang, Y. N., Poon, W., Tavares, A. J., McGilvray, I. D. & Chan, W. C. W. W. Nanoparticle-liver interactions: Cellular uptake and hepatobiliary elimination. J. Control. Release 240, 332-348 (2016).
33. Lalka, D., Griffith, R. K. & Cronenberger, C. L. The Hepatic First-Pass Metabolism of Problematic Drugs. J. Clin. Pharmacol. 33, 657-669 (1993).
34. Gao, H. & Matyjaszewski, K. Synthesis of molecular brushes by ‘grafting onto’ method: Combination of ATRP and click reactions. J. Am. Chem. Soc. 129, 6633-6639 (2007).
35. Ma, F. et al. Neurotransmitter-derived lipidoids (NT-lipidoids) for enhanced brain delivery through intravenous injection. Sci. Adv. 6, (2020).
36. Tanaka, H. et al. In Vivo Introduction of mRNA Encapsulated in Lipid Nanoparticles to Brain Neuronal Cells and Astrocytes via Intracerebroventricular Administration. Mol. Pharm. 15, 2060-2067 (2018).
37. Choi, B. et al. Development of novel, biocompatible, polyester amines for microglia-Targeting gene delivery. RSC Adv. 11, 36792-36800 (2021).
38. Dhaliwal, H. K., Fan, Y., Kim, J. & Amiji, M. M. Intranasal Delivery and Transfection of mRNA Therapeutics in the Brain Using Cationic Liposomes. Mol. Pharm. 17, 1996-2005 (2020).
39. Guo, S. et al. Specific Silencing of Microglial Gene Expression in the Rat Brain by Nanoparticle-Based Small Interfering RNA Delivery. ACS Appl. Mater. Interfaces 14, 5066-5079 (2022).
40. Mungenast, A. E., Siegert, S. & Tsai, L. H. Modeling Alzheimer's disease with human induced pluripotent stem (iPS) cells. Mol. Cell. Neurosci. 73, 13-31 (2016).
41. Muffat, J. et al. Efficient derivation of microglia-like cells from human pluripotent stem cells. Nat. Med. 22, 1358-1367 (2016).
42. Penney, J., Ralvenius, W. T. & Tsai, L. H. Modeling Alzheimer's disease with iPSC-derived brain cells. Molecular Psychiatry vol. 25 148-167 (2020).
43. Ellert-Miklaszewska, A. et al. Efficient and innocuous delivery of small interfering RNA to microglia using an amphiphilic dendrimer nanovector. Nanomedicine 14, 2441-2458 (2019).
44. Li, B., Luo, X. & Dong, Y. Effects of Chemically Modified Messenger RNA on Protein Expression. Bioconjug. Chem. 27, 849-853 (2016).
45. Lokugamage, M. P. et al. Mild Innate Immune Activation Overrides Efficient Nanoparticle-Mediated RNA Delivery. Adv. Mater. 32, 1904905 (2020).
46. Miao, L. et al. Synergistic lipid compositions for albumin receptor mediated delivery of mRNA to the liver. Nat. Commun. 11, (2020).
47. Peluffo, H. et al. BBB-targeting, protein-based nanomedicines for drug and nucleic acid delivery to the CNS. Biotechnology Advances vol. 33 277-287 (2015).
48. Rosenblum, D. et al. CRISPR-Cas9 genome editing using targeted lipid nanoparticles for cancer therapy. Sci. Adv. 6, eabc9450 (2020).
49. Rungta, R. L. et al. Lipid nanoparticle delivery of sirna to silence neuronal gene expression in the brain. Mol. Ther. —Nucleic Acids 2, e136 (2013).
50. Guimaraes, P. P. G. et al. Ionizable lipid nanoparticles encapsulating barcoded mRNA for accelerated in vivo delivery screening. J. Control. Release 316, 404-417 (2019).
51. Paunovska, K. et al. A Direct Comparison of in Vitro and in Vivo Nucleic Acid Delivery Mediated by Hundreds of Nanoparticles Reveals a Weak Correlation. Nano Lett. 18, 2148-2157 (2018).
52. Navath, R. S. et al. Injectable PAMAM Dendrimer-PEG Hydrogels for the Treatment of Genital Infections: Formulation and in Vitro and in Vivo Evaluation. Mol. Pharm. 8, 1209-1223 (2011).

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, books, manuals, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of selectively delivering an agent to a cell, comprising contacting the cell with a composition comprising an agent and lipids selected from: (a) an ionizable amino lipid, (b) a sterol, (c) a phospholipid, and (d) a PEG-lipid.

2. The method of claim 1, wherein the ionizable amino lipid is an ionizable amino alcohol lipid.

3. The method of claim 2, wherein the ionizable amino alcohol lipid is a compound of Formula (I): 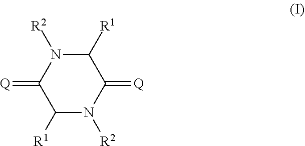
or salt thereof;
wherein:
each instance of Q is independently O, S, or NR^{Q}, wherein R^{Q} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), (iii);
each instance of R¹ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, halogen, —OR^{A1}, —N(R^{A1})₂, —SR^{A1}; wherein each occurrence of R^{A1} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to an sulfur atom, a nitrogen protecting group when attached to a nitrogen atom, or two R^{A1} groups are joined to form an optionally substituted heterocyclic or optionally substituted heteroaryl ring;
or at least one instance of R¹ is a group of formula: 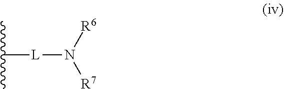
wherein L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, and
R⁶ and R⁷ are each independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group;
each instance of R² is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii); and
Formulae (i), (ii), and (iii) are: 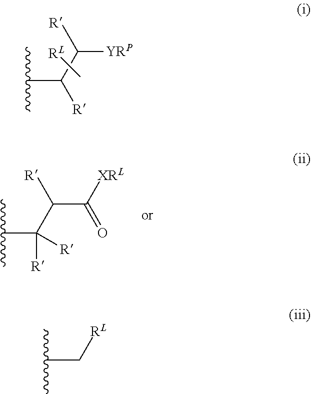
wherein:
each instance of R′ is independently hydrogen or optionally substituted alkyl;
X is O, S, NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
Y is O, S, NR, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{P} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and
R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer;
provided that at least one instance of R^{Q}, R², or R¹ comprise a group of the formula (i), (ii), or (iii).

4. The method of claim 3, wherein the ionizable amino lipid is selected from the group consisting of: 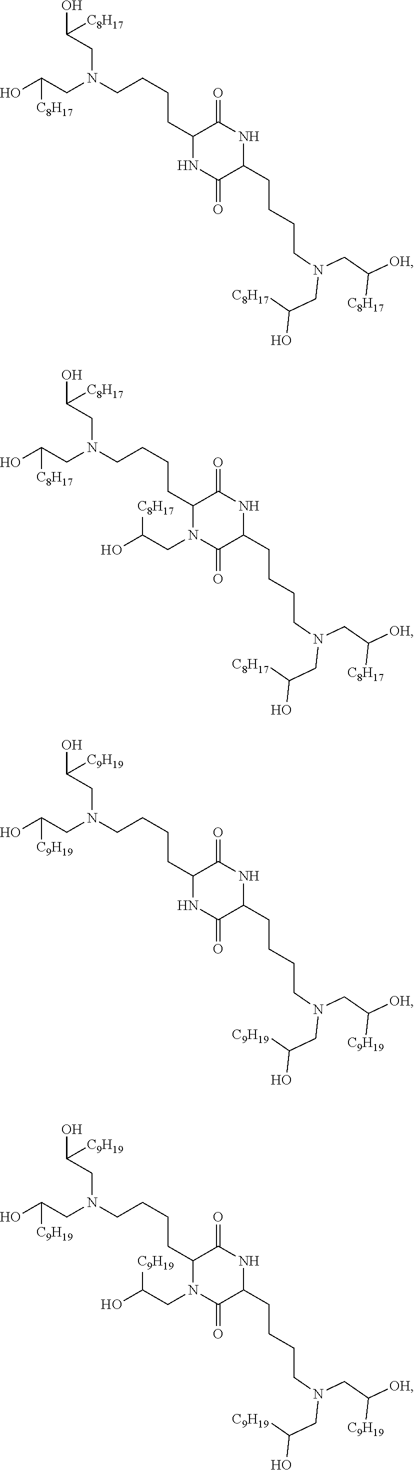 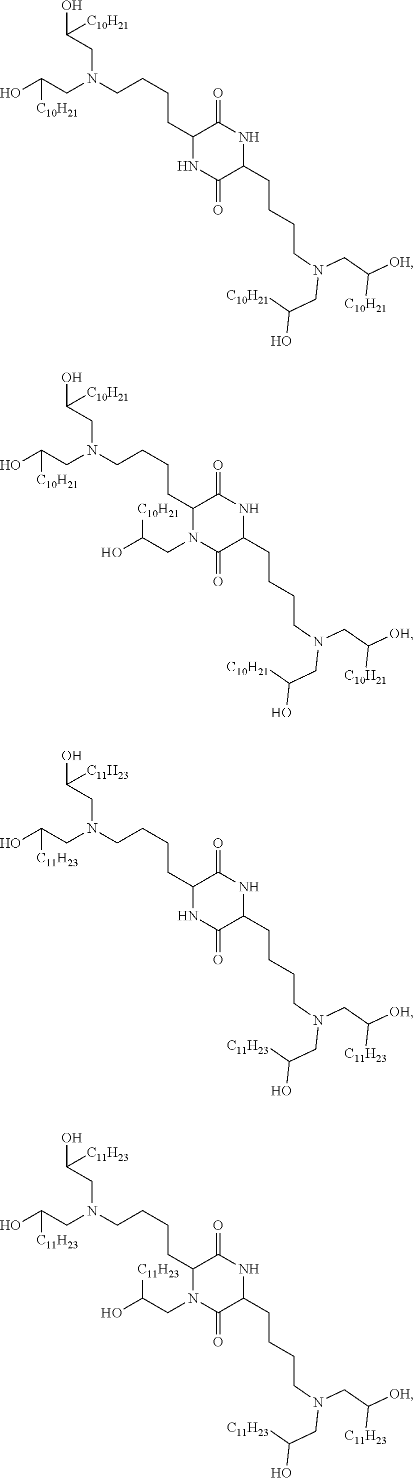 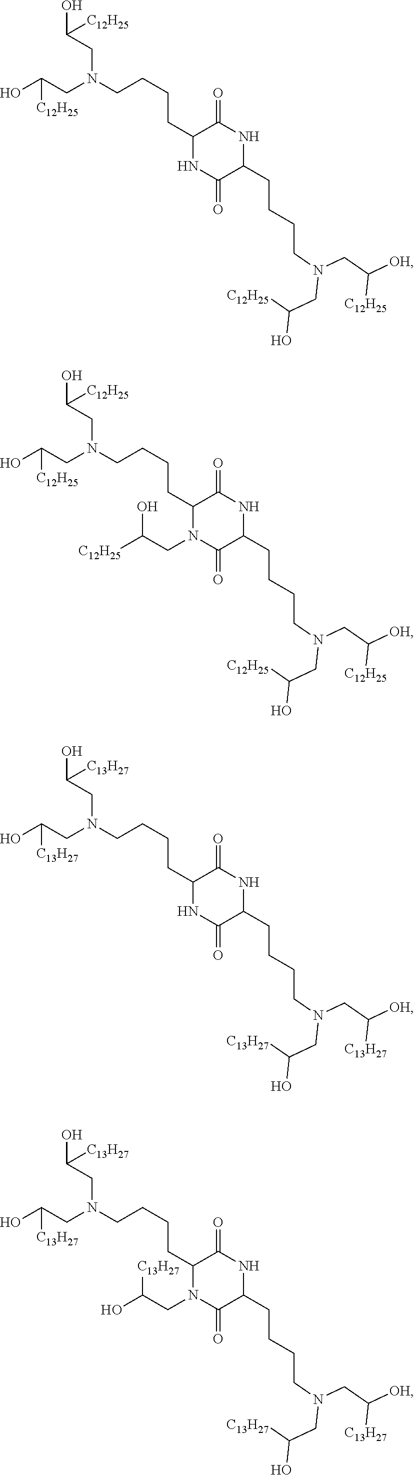 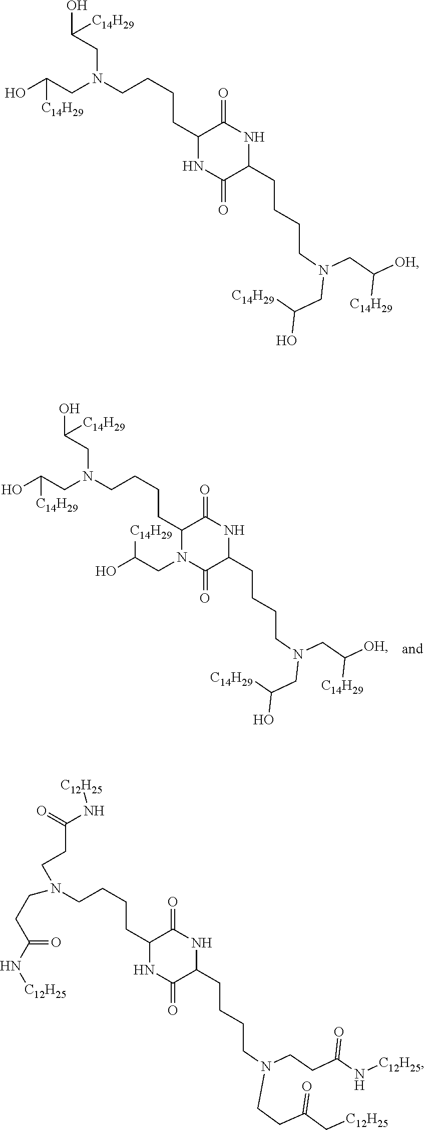
and salts thereof.

5. The method of any one of the preceding claims, wherein the sterol is cholesterol.

6. The method of any one of the preceding claims, wherein the phospholipid is 1,2-distearoyl-sn-glycero-3-phosphorylethanolamine (DSPE), 1,2-distearoyl-sn-glycero-3-phosphocholine (DSPC), or 1,2-dioleoyl-sn-glycero-3-phosphoethanolamine (DOPE).

7. The method of any one of the preceding claims, wherein the PEG-lipid is a PEG-phospholipid or PEG-glyceride lipid.

8. The method of claim 7, wherein the PEG-lipid is 1,2-dimyristoyl-sn-glycero-3-phosphoethanolamine-N-[methoxy(polyethylene glycol)-2000] (C₁₄PEG2000) or 1,2-dimyristoyl-rac-glycero-3-methoxypolyethylene glycol-2000 (DMG-PEG2000).

9. The method of claim 1, wherein the lipids are selected from (a) cKK-E12, (b) cholesterol, (c) DOPE, and (d) DMG-PEG2000.

10. The method of claim 1, wherein the lipids are selected from (a) cKK-E12, (b) cholesterol, (c) DOPE, and (d) C₁₄PEG2000.

11. The method of any one of claims 1-10, wherein the molar percentages of lipids are: (a) 35%, (b) 46.5%, (c) 16%, and (d) 2.5%.

12. The method of any one of the preceding claims, wherein the agent is an organic molecule, inorganic molecule, nucleic acid, protein, peptide, polynucleotide, targeting agent, an isotopically labeled chemical compound, vaccine, an immunological agent, or an agent useful in bioprocessing.

13. The method of claim 12, wherein the agent is a polynucleotide.

14. The method of claim 13, wherein the polynucleotide is mRNA.

15. The method of claim 14, wherein the weight ratio of ionizable amino lipid:mRNA is 10:1.

16. The method of claim 14, wherein the weight ratio of ionizable amino lipid:mRNA is 5:1.

17. The method of any one of claims 1-16, wherein the cell is a monocyte.

18. The method of any one of claims 1-16, wherein the cell is a macrophage.

19. The method of claim 18, wherein the macrophage is a microglial cell.

20. The method of claim 19, wherein the microglial cell is an inflammatory microglial cell.

21. The method of claim 20, wherein the delivery is selective for an inflammatory microglial cell over other neuroglia.

22. The method of claim 21, wherein the delivery is selective for an inflammatory microglial cell over an astrocyte.

23. A method of treating or preventing a disease, disorder, or condition in a subject in need thereof, comprising administering to the subject a composition comprising an agent, and lipids selected from: (a) an ionizable amino lipid, (b) a sterol, (c) a phospholipid, and (d) a PEG-lipid.

24. The method of claim 23, wherein the disease, disorder, or condition is a disease that is amenable to gene-delivery therapy.

25. The method of claim 23 or 24, wherein the disease, disorder, or condition is a neurological disease, an inflammatory disease or condition, an autoimmune disorder, cancer, or an injury.

26. The method of claim 25, wherein the disease, disorder, or condition is an inflammatory disease or condition.

27. The method of claim 25, wherein the disease, disorder, or condition is an autoimmune disorder.

28. The method of claim 25, wherein the disease, disorder, or condition is cancer.

29. The method of claim 25, wherein the disease, disorder, or condition is an injury.

30. The method of claim 25, wherein the disease, disorder, or condition is a neurological disease.

31. The method of claim 30, wherein the neurological disease is a neurodegenerative disease.

32. The method of claim 31, wherein the neurodegenerative disease is Alzheimer's disease.

33. The method of any one of claims 23-32, wherein the subject is a mammal.

34. The method of claim 33, wherein the subject is a human.

35. The method of any one of claims 23-34, wherein the method further comprises selectively delivering the agent to a cell.

36. The method of any one of claims 23-35, wherein the ionizable amino lipid is an ionizable amino alcohol lipid.

37. The method of claim 36, wherein the ionizable amino alcohol lipid is a compound of Formula (I): 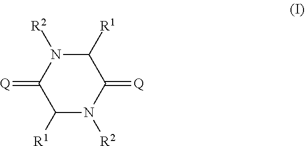
or salt thereof;
wherein:
each instance of Q is independently O, S, or NR^{Q}, wherein R^{Q} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), (iii);
each instance of R¹ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, halogen, —OR^{A1}, —N(R^{A1})₂, —SR^{A1}; wherein each occurrence of R^{A1} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to an sulfur atom, a nitrogen protecting group when attached to a nitrogen atom, or two R^{A1} groups are joined to form an optionally substituted heterocyclic or optionally substituted heteroaryl ring;
or at least one instance of R¹ is a group of formula: 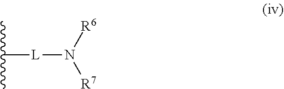
wherein L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene, and
R⁶ and R⁷ are each independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group;
each instance of R² is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or a group of the formula (i), (ii), or (iii); and
Formulae (i), (ii), and (iii) are: 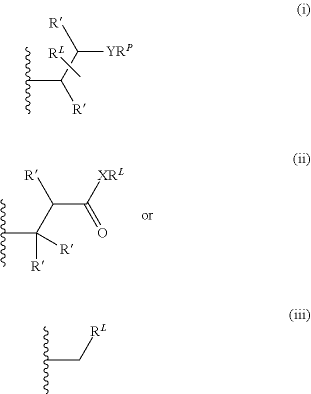
wherein:
each instance of R′ is independently hydrogen or optionally substituted alkyl;
X is O, S, NR^{X}, wherein R^{X} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
Y is O, S, NR, wherein R^{Y} is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, or a nitrogen protecting group when attached to a nitrogen atom; and
R^{L} is optionally substituted C₁₋₅₀ alkyl, optionally substituted C₂₋₅₀ alkenyl, optionally substituted C₂₋₅₀ alkynyl, optionally substituted heteroC₁₋₅₀ alkyl, optionally substituted heteroC₂₋₅₀ alkenyl, optionally substituted heteroC₂₋₅₀ alkynyl, or a polymer;
provided that at least one instance of R^{Q}, R², R⁶, or R⁷ is a group of the formula (i), (ii), or (iii).

38. The method of claim 37, wherein the ionizable amino lipid is selected from the group consisting of: 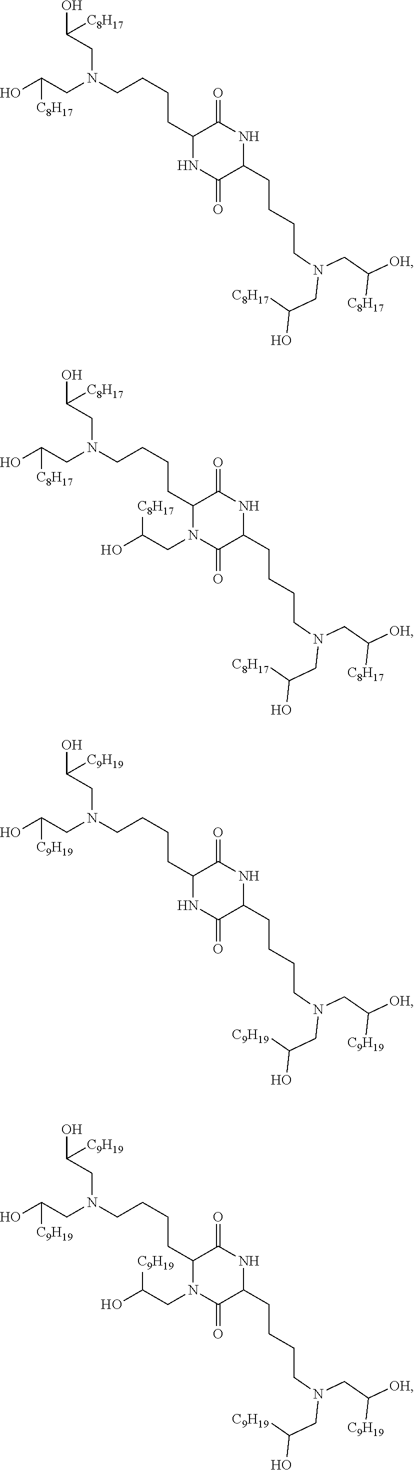 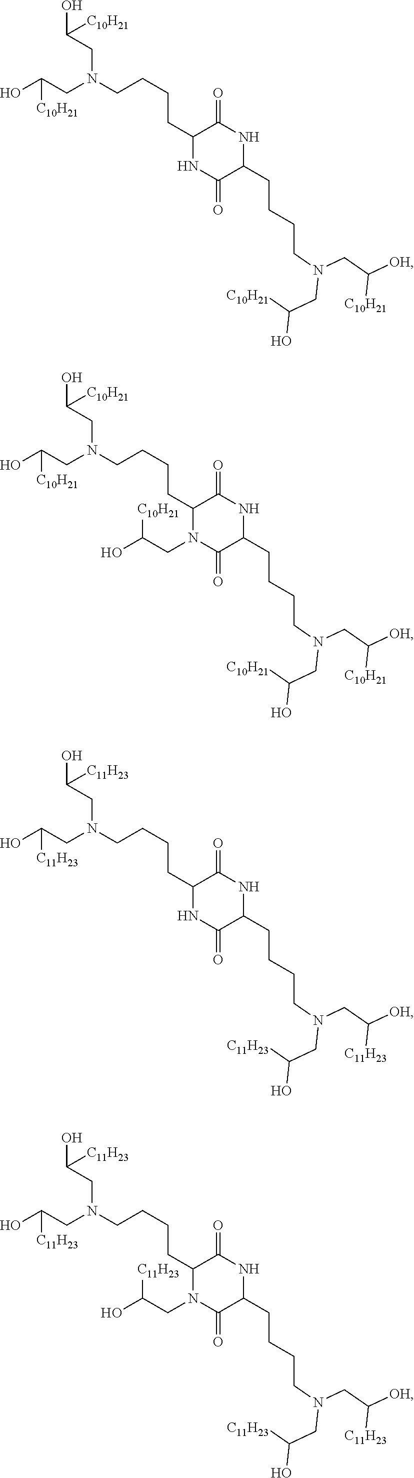 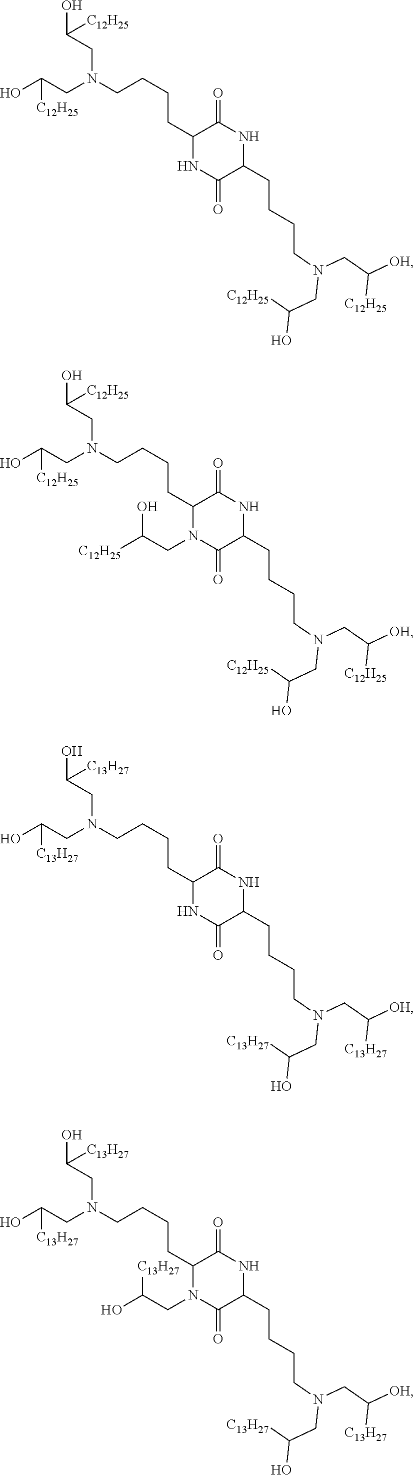 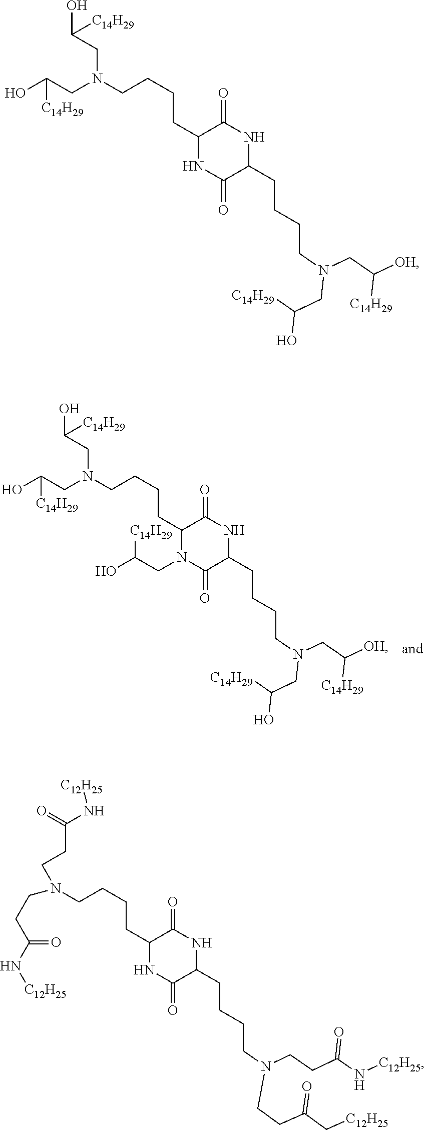
and salts thereof.

39. The method of any one of claims 23-38, wherein the sterol is cholesterol.

40. The method of any one of claims 23-39, wherein the phospholipid is 1,2-distearoyl-sn-glycero-3-phosphorylethanolamine (DSPE), 1,2-distearoyl-sn-glycero-3-phosphocholine (DSPC), or 1,2-dioleoyl-sn-glycero-3-phosphoethanolamine (DOPE).

41. The method of any one of claims 23-40, wherein the PEG-lipid is a PEG-phospholipid or PEG-glyceride lipid.

42. The method of claim 41, wherein the PEG-lipid is 1,2-dimyristoyl-sn-glycero-3-phosphoethanolamine-N-[methoxy(polyethylene glycol)-2000] (C₁₄PEG2000) or 1,2-dimyristoyl-rac-glycero-3-methoxypolyethylene glycol-2000 (DMG-PEG2000).

43. The method of any one of claims 23-42, wherein the lipids are selected from (a) cKK-E12, (b) cholesterol, (c) DOPE, and (d) DMG-PEG2000.

44. The method of any one of claims 23-42, wherein the lipids are selected from (a) cKK-E12, (b) cholesterol, (c) DOPE, and (d) C₁₄PEG2000.

45. The method of any one of claims 23-44, wherein the molar percentages of the lipids are: (a) 35%, (b) 46.5%, (c) 16%, and (d) 2.5%.

46. The method of any one of claims 23-45, wherein the agent is an organic molecule, inorganic molecule, nucleic acid, protein, peptide, polynucleotide, targeting agent, an isotopically labeled chemical compound, vaccine, an immunological agent, or an agent useful in bioprocessing.

47. The method of claim 46, wherein the agent is a polynucleotide.

48. The method of claim 47, wherein the polynucleotide is mRNA.

49. The method of claim 48, wherein the weight ratio of ionizable amino lipid:mRNA is 10:1.

50. The method of claim 48, wherein the weight ratio of ionizable amino lipid:mRNA is 5:1.

51. The method of any one of claims 35-50, wherein the cell is a monocyte.

52. The method of any one of claims 35-50, wherein the cell is a macrophage.

53. The method of claim 52, wherein the macrophage is a microglial cell.

54. The method of claim 53, wherein the microglial cell is an inflammatory microglial cell.

55. The method of claim 54, wherein the delivery is selective for an inflammatory microglial cell over other neuroglia.

56. The method of claim 55, wherein the delivery is selective for an inflammatory microglial cell over an astrocyte.
